(19) United States
(12) Patent Application Publication
(10) Pub. No.: US 2020/0255846 A1
(43) Pub. Date: Aug. 13, 2020

(54) METHODS FOR INCREASING GRAIN YIELD

(71) Applicant: Institute of Genetics and Developmental Biology Chinese Acedemy of Sciences, Beijing (CN)

(72) Inventors: Penggen Duan, Beijing (CN); Jinsong Xu, Beijing (CN); Yunhai Li, Beijing (CN)

(21) Appl. No.: 16/497,161

(22) PCT Filed: Mar. 23, 2018
(86) PCT No.: PCT/GB2018/050761

§ 371 Date: Sep. 24, 2019

(30) Foreign Application Priority Data
Mar. 24, 2017 (CN) PCT/CN2017/078137

(51) Int. Cl. C12N 15/82; C07K 14/415

(52) U.S. Cl. CPC C12N 15/8213; C07K 14/415; C12N 15/8261; C12N 15/8218

(57) ABSTRACT

The invention relates to methods for increasing plant yield, and in particular seed yield by reducing the expression of GSE5 or GSE5-Like in a plant. Also described are genetically altered plants characterised by the above phenotype and methods of producing such plants.

### FIELD OF THE INVENTION

The invention relates to methods for increasing plant yield, and in particular seed yield by reducing the expression of GSE5 or GSE5-Like in a plant. Also described are genetically altered plants characterised by the above phenotype and methods of producing such plants.

### BACKGROUND OF THE INVENTION

Modern agriculture must meet the challenges of feeding an increasing population and decreasing arable land. Rice is an important crop, providing food for more than half the global population. The genetic variation in diverse rice varieties provides a valuable resource to improve important agronomic traits in rice. Rice breeders have explored natural variation in genes involved in the regulation of yield-related traits to develop elite rice varieties (Zuo and Li, 2014). Rice grain yield is determined by grain weight, grain number per panicle and panicle number per plant. Grain size is associated with grain weight, grain yield and appearance quality. Several QTL genes for grain size have been identified in rice (Che et al., 2015; Duan et al., 2015; Fan et al., 2006; Hu et al., 2015; Ishimaru et al., 2013; Li et al., 2011; Qi et al., 2012; Shomura et al., 2008; Si et al., 2016; Song et al., 2007; Wang et al., 2015a; Wang et al., 2012; Wang et al., 2015b; Weng et al., 2008; Zhang et al., 2012), but only a few of these beneficial alleles are widely utilized by rice breeders (Li and Li, 2016; Zuo and Li, 2014).

Asian cultivated rice includes indica and japonica subspecies, which show large variation in grain size and shape. Typical indica varieties produce long grains, whereas japonica varieties form round and short grains. Natural variation in several genes has been reported to be selected by rice breeders. For example, natural variation in the major QTL for grain length (GS3) contributes to grain-length differences between indica varieties and japonica varieties (Fan et al., 2006; Mao et al., 2010). The indica varieties with long grains usually contain a loss-of-function allele, while japonica varieties with short grains often have the wild-type allele. By contrast, the major QTL gene for grain width (qSW5/GW5) influences grain-width differences between indica varieties and japonica varieties. The qSW5/GW5 encodes an unknown protein (Shomura et al., 2008; Weng et al., 2008). The 1212-bp deletion in most japonica varieties disrupts the qSW5 gene, resulting in wide grains. By contrast, some indica varieties do not contain this 1212-bp deletion in the qSW5 gene, thereby producing narrow grains (Weng et al., 2008). Genome-wide association studies (GWAS) have identified multiple association signals for grain size in cultivated rice (Huang et al., 2010). The QTL gene GLW7/OsSPL13 has been recently identified using the GWAS approach (Si et al., 2016). High expression of GLW7 is associated with large grains in tropical japonica rice. However, the grain size genes underlying natural variation have not been fully explored in rice.

Here we identify a novel quantitative trait locus for grain size (GSE5) using a genome-wide association study with functional testing. GSE5 encodes a plasma membrane associated protein with IQ domains (IQD), which regulates grain width by restricting cell proliferation. Loss-of-function of GSE5 increases grain width, while overexpression of GSE5 results in slender grains. Two major type deletions (DEL1 and DEL2) happen in the promoter region of GSE5 in some indica varieties and most japonica varieties, respectively, resulting in the decreased expression of GSE5 and wide grains. DEL1 and DEL2 are widely utilized in indica and japonica rice production, respectively. Wild rice accessions contain DEL1 and DEL2, suggesting that these two deletions in cultivated rice are likely to have originated from different wild rice accessions during rice domestication. We have also identified a GSE5-Like protein, that has 72.5% identity with GSE5 and that similarly, reducing the expression of GSE5-Like increases grain length, grain width and yield. Thus, our findings provide insight into a natural variation in grain size control.

As seed yield is a major factor in determining the commercial success of grain crops it is important to not only understand the genetic factors that underlie this trait, but also how to modulate such factors to improve overall grain yield. The present invention addresses this need.

### SUMMARY OF THE INVENTION

The inventors have surprisingly identified that the expression of GSE5 or GSE5-Like correlates negatively with the yield component traits, grain weight, grain width and thousand kernel weight (TKW) across Oryza sativa accessions. Accordingly, the inventors have surprisingly shown that reducing the level of GSE5 or GSE5-Like expression and/or the activity of the GSE5 or GSE5-Like polypeptide can significantly increase grain yield.

In one aspect of the invention there is provided a method of increasing yield in a plant, the method comprising reducing or abolishing the expression of at least one (grain size on chromosome 5) GSE5 or GSE5-Like nucleic acid and/or reducing the activity of a GSE5 or GSE5-Like polypeptide in said plant. In one embodiment, the method may comprise reducing or abolishing the expression of at least one GSE5 and GSE5-Like nucleic acid and/or reducing the activity of a GSE5 and GSE5-Like polypeptide in said plant.

In one embodiment, said increase is an increase in grain yield. Preferably, said increase in grain yield is preferably an increase in at least one of grain weight, grain width and/or thousand kernel weight.

In one embodiment, the method comprises introducing at least one mutation into the nucleic acid sequence encoding GSE5 or GSE5-Like or at least one mutation into the promoter of GSE5 or GSE5-Like. Preferably, said mutation is a loss of function or partial loss of function mutation. More preferably, said mutation is an insertion, deletion and/or substitution.

In one embodiment, the GSE5 nucleic acid encodes a polypeptide comprising SEQ ID NO: 1 or a functional variant or homolog thereof. Preferably, the GSE5 nucleic acid comprises SEQ ID NO: 2 or a functional variant or homolog thereof. In another embodiment, the GSE5-Like nucleic acid encodes a polypeptide comprising SEQ ID NO: 57 or a functional variant or homolog thereof. Preferably, the GSE5-Like nucleic acid comprises SEQ ID NO: 55 or 56 or a functional variant or homolog thereof.

In another embodiment, the GSE5 promoter comprises a nucleic acid sequence as defined in SEQ ID NO: 28 or a functional variant or homolog thereof.

In one embodiment, the mutation is introduced using targeted genome modification, preferably ZFNs, TALENs or CRISPR/Cas9. In an alternative embodiment, the mutation is introduced using mutagenesis, preferably TILLING or T-DNA insertion. In a further alternative embodiment, the method comprises using RNA interference to reduce or abolish the expression of a GSE5 nucleic acid and/or reduce or abolish the activity of a GSE5 or GSE5-Like promoter.

In one embodiment, said increase in seed yield is relative to a control or wild-type plant.

In another aspect of the invention, there is provided a genetically modified plant, plant cell or part thereof characterised by a reduced level of GSE5 or GSE5-Like nucleic acid expression and/or reduced activity of the GSE5 or GSE5-Like polypeptide.

In one embodiment, said plant is characterised by an increase in yield compared to a wild-type on control pant. Preferably, said increase in yield is an increase in at least grain yield. More preferably, said increase in grain yield is preferably an increase in at least one of grain weight, grain width and/or thousand kernel weight.

In one embodiment, said plant comprises at least one mutation in at least one nucleic acid sequence encoding GSE5 or GSE5-Like or at least one mutation in the promoter of GSE5 or GSE5-Like. Preferably, said mutation is a loss of function or partial loss of function mutation. More preferably, said mutation is an insertion, deletion and/or substitution.

In one embodiment, the GSE5 nucleic acid encodes a polypeptide comprising of SEQ ID NO: 1 or a functional variant or homolog thereof. Preferably, the GSE5 nucleic acid comprises SEQ ID NO: 2 or 32 or a functional variant or homolog thereof. In another embodiment, the GSE5-Like nucleic acid encodes a polypeptide comprising SEQ ID NO: 57 or a functional variant or homolog thereof. Preferably, the GSE5-Like nucleic acid comprises SEQ ID NO: 55 or 56 or a functional variant or homolog thereof.

In another embodiment, the GSE5 promoter comprises a nucleic acid sequence as defined in SEQ ID NO: 28 or a functional variant or homolog thereof.

In one embodiment, the mutation is introduced using targeted genome modification, preferably ZFNs, TALENs or CRISPR/Cas9. In another embodiment, the mutation is introduced using mutagenesis, preferably TILLING or T-DNA insertion. In a further alternative embodiment, the plant comprises an RNA interference construct that reduces or abolishes the expression of a GSE5 or GSE5-Like nucleic acid and/or reduces or abolishes the activity of a GSE5 or GSE5-Like promoter.

In one embodiment, the plant part is a seed.

In another aspect of the invention, there is provided a method of producing a plant with increased yield, the method comprising introducing at least one mutation into at least one nucleic acid sequence encoding GSE5 or GSE5-Like and/or at least one mutation in the promoter of GSE5 or GSE5-Like. Preferably, the mutation is a loss of function or partial loss of function mutation. More preferably, the mutation is an insertion, deletion and/or substitution.

In one embodiment, the mutation is introduced using mutagenesis or targeted genome modification. Preferably, the targeted genome modification is selected from ZFNs, TALENs or CRISPR/Cas9.

In one embodiment, mutagenesis is selected from TILLING or T-DNA insertion.

In another aspect of the invention, there is provided a plant, plant part or plant cell obtained by the method described herein. In a further aspect of the invention, there is provided a seed obtained or obtainable from the plant as described herein or the method as described herein.

In a further aspect of the invention, there is provided a method for identifying and/or selecting a plant that will have an increased seed yield phenotype, the method comprising detecting in the plant or plant germplasm at least one mutation in the promoter of the GSE5 or GSE5-Like gene, wherein said plant or progeny thereof is selected.

In one embodiment, the mutation is an insertion and/or deletion. Preferably, the mutation is the deletion of a nucleic acid sequence comprising SEQ ID NO: 29 (DEL1) or SEQ ID NO: 30 (DEL2). Alternatively or additionally, the mutation is the insertion of a nucleic acid sequence comprising SEQ ID NO: 31 (IN1).

In a further embodiment, the method further comprises introgressing the chromosomal region comprising at least one of said polymorphisms and/or deletions into a second plant or plant germplasm to produce an introgressed plant or plant germplasm.

In another aspect of the invention, there is provided a nucleic acid construct comprising a nucleic acid sequence encoding at least one DNA-binding domain that can bind to at least one GSE5 gene or GSE5-Like, wherein said sequence is selected from SEQ ID NOs: 15 to 20, 48, 51, 76 and 79 to 84.

In one embodiment, the nucleic acid sequence encodes at least one protospacer element, and wherein the sequence of the protospacer element is selected from SEQ ID NOs: 21 to 26 or 52 or 77 or a sequence that is at least 90% identical to SEQ ID NOs: 21 to 26 or 52 or 77.

In a further embodiment, the construct further comprises a nucleic acid sequence encoding a CRISPR RNA (crRNA) sequence, wherein said crRNA sequence comprises the protospacer element sequence and additional nucleotides.

In another embodiment, the construct further comprises a nucleic acid sequence encoding a transactivating RNA (tracrRNA).

In a further embodiment, the construct encodes at least one single-guide RNA (sgRNA), wherein said sgRNA comprises the tracrRNA sequence and the crRNA sequence, wherein the sgRNA.

Preferably the nucleic acid encoding a DNA-binding domain, protospacer element, crRNA, tracrRNA or sgRNA is operably linked to a promoter. Preferably, the promoter is a constitutive promoter.

In a further embodiment, the nucleic acid construct further comprises a nucleic acid sequence encoding a CRISPR enzyme. Preferably, the CRISPR enzyme is a Cas protein. More preferably, the Cas protein is Cas9 or a functional variant thereof.

In an alternative embodiment, the nucleic acid construct encodes a TAL effector. Preferably, the nucleic acid construct further comprises a sequence encoding an endonuclease or DNA-cleavage domain thereof. More preferably, the endonuclease is Fokl.

In another aspect of the invention there is provided a single guide (sg) RNA molecule wherein said sgRNA comprises a crRNA sequence and a tracrRNA sequence, wherein the crRNA sequence can bind to at least one sequence selected from SEQ ID NOs: 15 to 20, 48, 51, 76 or 79 to 84.

In a further aspect of the invention there is provided an isolated plant cell transfected with at least one nucleic acid construct as described herein.

In an alternative aspect of the invention there is provided an isolated plant cell transfected with at least a first nucleic acid construct as described herein (comprising nucleic acid encoding a sgRNA) and a second nucleic acid construct, wherein said second nucleic acid construct comprising a nucleic acid sequence encoding a Cas protein, preferably a Cas9 protein or a functional variant thereof. Preferably, the second nucleic acid construct is transfected before, after or concurrently with the first nucleic acid construct.

In another aspect of the invention, there is provided a genetically modified plant, wherein said plant comprises the transfected cell above. In one embodiment, the nucleic acid encoding the sgRNA and/or the nucleic acid encoding a Cas protein is integrated in a stable form.

In a further aspect of the invention, there is provided a nucleic acid construct comprising a nucleic acid sequence encoding a polypeptide as defined in SEQ ID NO: 1 or a functional variant or homolog thereof, wherein said sequence is operably linked to a regulatory sequence, wherein preferably said regulatory sequence is a tissue-specific promoter.

In another aspect of the invention there is provided a vector comprising the nucleic acid construct as described herein. In a further aspect, there is provided a host cell comprising the nucleic acid construct as described herein. In a yet further aspect, there is provided a transgenic plant expressing the nucleic acid construct as described herein.

In another aspect of the invention, there is provided a method of increasing grain length, the method comprising introducing and expressing in said plant the nucleic acid construct as described herein, wherein said increase is relative to a control or wild-type plant.

In a further aspect, there is provided a method for producing a plant with increased grain length, the method comprising introducing and expressing in said plant the nucleic acid construct as described herein, wherein said increase is relative to a control or wild-type plant.

In another aspect, there is provided a plant obtained or obtainable by the method as described herein.

In another aspect of the invention, there is provided the use of a nucleic acid construct as described herein to modulate the expression levels of at least one GSE5 or GSE5-Like nucleic acid in a plant. Preferably said nucleic acid construct reduces the expression levels of at least one GSE5 or GSE5-Like nucleic acid in a plant. Alternatively, said nucleic acid construct increases the expression levels of at least one GSE5 or GSE5-Like nucleic acid in a plant.

In a final aspect of the invention, there is provided a method for obtaining the genetically modified plant as described above, the method comprising:
a. selecting a part of the plant;
b. transfecting at least one cell of the part of the plant of paragraph (a) with the nucleic acid construct as described above;
c. regenerating at least one plant derived from the transfected cell or cells; selecting one or more plants obtained according to paragraph (c) that show silencing or reduced expression of at least one GSE5 or GSE5-Like nucleic acid in said plant.

In one embodiment of any of the above aspects, the plant is a crop plant. Preferably, the crop plant is selected from rice, wheat, maize, soybean and sorghum. More preferably, the crop plant is rice, preferably the japonica or indica variety.

### DESCRIPTION OF THE FIGURES

The invention is further described in the following non-limiting figures:

FIG. 1 shows the identification of a novel locus for grain size (GSE5) using a GWAS study with expression analysis.

(a) Genome-wide association study of grain width. Manhattan plots for grain width. Dashed line represents the significance threshold (P=2.78×10⁻⁵). The arrows indicate the loci for grain width.

(b) Q local manhattan plot (top) and LD heatmap (bottom) surrounding the peak on Chromosome 5. Dashed lines indicate the candidate region for the peak.

(c) The schematic diagram of the 22.42-kb genomic region. This region contains qSW5 and LOC_Os05g09520. Most japonica varieties have a 1212-bp deletion (DEL2) in the qSW5 gene. Some indica varieties have no deletion in qSW5, while some indica varieties contain a 950-bp deletion (DEL1) in the 3′ flanking region of qSW5, a 367-bp insertion (IN1) in the 5′ flanking region of LOC_Os05g09520 and a nucleotide change (G/A) in the first exon of LOC_Os05g09520. The arrow shows the direction of the qSW5 transcription. The red dash lines represent the deletions in the genomic regions.

(d) Comparison of qSW5 expression in young panicles of indica varieties without (1) or with (2) the 950-bp deletion (DEL1) in the 3′ flanking region of qSW5 (n=34/36).

(e) Correlation of the 950-bp deletion (DEL1) and 367-bp insertion (IN1) with grain width. Mature grains from the indica varieties without (1) or with DEL1+IN1 (2) were measured (n=68/65).

Values (d and e) are means±SD. Significance is determined using analysis of variance (ANOVA) (**P<0.01).

FIG. 2 shows that the DEL1 in indica varieties and DEL2 in japonica varieties cause the decreased expression of GSE5.

(a) Comparison of LOC_Os05g09520 expression in young panicles of narrow grain (NGV) and wide grain (WGV) indica varieties. Values are means±SD (n=20/20). Significance is determined using analysis of variance (ANOVA) (*P<0.05).

(b) Comparison of LOC_Os05g09520 expression in young panicles of rice varieties without (1) or with DEL1+IN1 (2) and DEL2 (3). Values are means±SD (n=34/36/31). Significance is determined using analysis of variance (ANOVA) (*P<0.05).

(c) Expression levels of LOC_Os05g09520 expression in young panicles of the japonica variety Nipponbare (NIP) with DEL2 and its near isogenic line (NIL). NIL contains the LOC_Os05g09520 allele from the narrow grain indica variety 93-11 in the japonica variety Nipponbare background. Values are means±SE (n=3). Significance is determined using t-test (**P<0.01).

(d) The constructs for each of the promoter-luciferase (LUC) fusions are shown. The arrow shows the direction of the qSW5 transcription.

(e) Effects of DEL1, IN1 and DEL2 on the activity of the GSE5 promoter. N. benthamiana leaves were transformed by injection of Agrobacterium GV3101 cells harboring proGSE5:LUC (1), proGSE5^{DEL1+IN1}:LUC (2), proGSE5^{DEL1}:LUC (3) and proGSE5^{DEL2}:LUC (4) plasmids, respectively. Relative reporter activity (LUC/REN) was calculated, and the value for proGSE5:LUC sets at 100. Values are means±SE (n=3). Significance is determined using t-test (**P<0.01).

FIG. 3 shows the identity of the GSE5 gene.

(a) The GSE5-cr mutant was generated by CRISPR/Cas9. In GSE5-cr mutant, the 1-bp deletion happens in the first exon of GSE5, resulting in a reading frame shift.

(b) Grains of Zhonghua 11 (ZH11) (left) and GSE5-cr (right).

(c-e) Grain width (c), grain length (d) and thousand grain weight (e) of Zhonghua 11 (ZH11) and GSE5-cr.

(f) Grains of Zhonghua 11 (ZH11) (left) and proActin:GSE5 (right). GSE5 was overexpressed in ZH11 background.

(g, h) Grain width (g) and grain length (h) of Zhonghua 11 (ZH11) and proActin:GSE5. GSE5 was overexpressed in ZH11 background.

(i, j) Grain width (i) and grain length (j) of Nipponbare (NP) and a near isogenic line (NIL), which contains the GSE5 locus from the narrow grain indica variety 93-11 in the japonica variety Nipponbare background.

Values (c-e, g-j) are means±SE. Significance is determined using t-test (**P<0.01). Bars=1 mm in b, f.

FIG. 4 shows how GSE5 controls grain size mainly by influencing cell proliferation.

(a, b) The outer epidermal surface of ZH11 (a) and GSE5-cr (b).

(c, d) The outer epidermal cell width (c) and the calculated outer epidermal cell number

(d) of ZH11 and GSE5-cr lemma in the grain-width direction.

(e, f) The outer epidermal cell width (e) and the calculated outer epidermal cell number

(f) of ZH11 and proActin:GSE5 (OE) lemma in the grain-width direction.

(g, h) The outer epidermal cell length (g) and the calculated outer epidermal cell number (h) of ZH11 and proActin:GSE5 (OE) lemma in the grain-length direction.

Values (c-h) are means±SE. Significance is determined using t-test (**P<0.01). Bars=100 μm in a and b.

FIG. 5 shows GSE5 encodes a plasma membrane associated protein with IQ domains (IQD).

(a) The GSE5 protein contains two IQ motifs and an unknown DUF4005 domain.

(b) The bimolecular fluorescence complementation (BiFC) assays show that GSE5 associated with OsCaM1-1 in N. benthamiana. nYFP-OsCaM1-1 and cYFP-GSE5 were coexpressed in leaves of N. benthamiana.

(c) Quantitative real-time RT-PCR analysis of GSE5 expression in young panicles of 5 cm (YP5), 10 cm (YP10), 15 cm (YP15) and 20 cm (YP20). Values are given as mean±SE (n=3).

(d-h) GSE5 expression activity was monitored using proGSE5:GSE5-GUS transgenic plants. GUS activity was detected in developing panicles.

(i) Subcellular localization of GSE5-GFP in proGSE5:GSE5-GFP transgenic plants. GFP fluorescence in proGSE5:GSE5-GFP transgenic plants was detected in the cell periphery. FM4-64 was used to stain the membrane.

(j) Cells were plasmolysed with 30% sucrose. GSE5-GFP was detected in the shrunken plasma membrane. FM4-64 was used to stain the membrane.

Bars=50 μm in b, 1 mm in d and e, 1 cm in f and g, 5 cm in h, and 10 μm in i and j.

FIG. 6 shows the evolutionary aspects of the GSE5 locus. (a, b) The percentages of GSE5, GSE5^{DEL1+IN1} and GSE5^{DEL2} haplotypes in indica and japonica varieties, respectively. 141 indica varieties and 91 japonica varieties were genotyped.

(c) Geographical origin of wild rice accessions used in this study. Wild rice accessions (O. rufipogon) contained GSE5, GSE5^{DEL1+IN1} and GSE5^{DEL2} haplotypes.

(d) Phylogenetic tree. The approximate 8.4 kb sequences including 6320-bp 5′ flanking sequence, the GSE5 gene and 1580-bp 3′ flanking sequence from 63 cultivated rice with GSE5, GSE5^{DEL1+IN1} and GSE5^{DEL2} haplotypes and 26 O. rufipogon with GSE5, GSE5^{DEL1+IN1} and GSE5^{DEL2} haplotypes were used to construct phylogenetic tree. Bootstrap values over 60% are given on the branches. The red letters represent O. rufipogon accession.

FIG. 7 shows grain size variation among 102 indica varieties. Frequency distributions of grain width (a) and grain length (b).

FIG. 8 shows a phylogenetic tree of GSE5 and its homologs. Neighbour-joining method of MEGA7.0 program was used to construct the phylogenetic tree of GSE5 homologs. Numbers at nodes indicate percentage of 1000 bootstrap replicates. The scale bar at the bottom represents genetic distance.

FIG. 9 shows an alignment of GSE5 and its rice homologue GSE5L1. The asterisk indicates identical amino acid residues. A colon represents conserved substitutions. A period shows semiconserved substitutions.

FIG. 10 shows that proGSE5:GSE5-GFP and proGSE5:GSE5-GUS transgenic plants produce narrow grains. Grain width of Zhonghua 11 (ZH11), proGSE5:GSE5-GFP and proGSE5:GSE5-GUS transgenic plants. proGSE5:GSE5-GFP and proGSE5:GSE5-GUS were transformed into the japonica variety ZH11.

Values are given as mean±SE. **P<0.01 compared with parental line (ZH11) by Student's t-test.

FIG. 11 shows a list of primers used in the study.

FIG. 12 shows A: Grain yield per plant of Zhonghua 11, GSE5-cr and proActin:GSE5 plants (n≥12). GSE5 was overexpressed in Zhonghua 11 background. B: Grains of Zhonghua 11 (left) and GSE5-Like-crispr (right). C, D: Grain length (C) and grain width (D) of Zhonghua 11 and GSE5-Like-crispr. Values (A, C, D) are means±SE. Significance is determined using t-test (*P<0.05). Bars=1 mm in B.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of botany, microbiology, tissue culture, molecular biology, chemistry, biochemistry and recombinant DNA technology, bioinformatics which are within the skill of the art. Such techniques are explained fully in the literature.

As used herein, the words “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences.

The terms “polypeptide” and “protein” are used interchangeably herein and refer to amino acids in a polymeric form of any length, linked together by peptide bonds.

The aspects of the invention involve recombination DNA technology and exclude embodiments that are solely based on generating plants by traditional breeding methods.

### Methods of Increasing Yield

Accordingly, in a first aspect of the invention, there is provided a method of increasing yield in a plant, the method comprising reducing or abolishing the expression of at least one nucleic acid encoding a grain size on chromosome 5 (referred to herein as GSE5) or GSE5-Like polypeptide and/or reducing the activity of a GSE5 polypeptide or GSE5-Like polypeptide in said plant. In one embodiment, the method may comprise reducing or abolishing the expression of at least one GSE5 and GSE5-Like nucleic acid and/or reducing the activity of a GSE5 and GSE5-Like polypeptide in said plant.

The term “yield” in general means a measurable produce of economic value, typically related to a specified crop, to an area, and to a period of time. Individual plant parts directly contribute to yield based on their number, size and/or weight. Alternatively, the actual yield is the yield per square meter for a crop and year, which is determined by dividing total production (includes both harvested and appraised production) by planted square meters. Preferably, in the present context, the term “yield” of a plant relates to propagule generation (such as seeds) of that plant. Thus, in a preferred embodiment, the method relates to an increase in seed yield or total seed yield.

The terms “seed” and “grain” as used herein can be used interchangeably.

According to the invention, seed yield can be measured by assessing one or more of seed weight, seed size, seed number per pod, seed number per plant, pod length, seed protein, a combination of both seed size and seed number and/or lipid content and weight of seed per pod. However, seed width and weight are some of the main components that contribute to seed yield. Therefore, in one embodiment an increase in seed yield comprises an increase in seed biomass or seed weight, which may be an increase in the seed weight per plant or in an increase in individual seed weight, an increase in seed width (individual or as an average over the whole plant) and/or an increase in thousand kernel weight (TKW), which can be extrapolated from the number of filled seeds counted and their total weight. An increase in the TKW can result from an increase in seed size and/or seed weight. Preferably, an increase in seed yield is an increase in at least one of seed weight, seed width and TKW. Yield is increased relative to control plants. The skilled person would be able to measure any of the above seed yield parameters using known techniques in the art.

The terms “increase”, “improve” or “enhance” as used herein are interchangeable. In one embodiment, seed yield, and preferably seed weight, seed width and/or the TKW are increased by at least 2%, 3%, 4%, 5%, 6%, 7%, 8%, 9%, 10% 11%, 12%, 13%, 14%, 15%, 16%, 17%, 18%, 19%, 20%, 30%, 40%, 50%, 55%, 60%, 65%, 70%, 75%, 80%, 85%, 90%, 95%, 100%, 105%, 110%, 120% or more in comparison to a control plant. Preferably, the increase is at least 2-10%, more preferably 3-8%. These increases can be measured by any standard technique known to the skilled person. In one embodiment, seed width is increased by more than 100%, preferably at least 110% or more compared to a control phenotype.

The terms “reducing” means a decrease in the levels of GSE5 or GSE5-Like polypeptide expression and/or activity by up to 10%, 20%, 30%, 40%, 50%, 60%, 70%, 80% or 90% when compared to the level in a wild-type or control plant. In a preferred embodiment, said decrease is at least 30%. The term “abolish” expression means that no expression of GSE5 or GSE5-Like polypeptide is detectable or that no functional GSE5 or GSE5-Like polypeptide is produced. Method for determining the level of GSE5 or GSE5-Like polypeptide expression and/or activity would be well known to the skilled person. These reductions can be measured by any standard technique known to the skilled person. For example, a reduction in the expression and/or content levels of at least GSE5 or GSE5-Like expression may be a measure of protein and/or nucleic acid levels and can be measured by any technique known to the skilled person, such as, but not limited to, any form of gel electrophoresis or chromatography (e.g. HPLC).

By “at least one mutation” is means that where the GSE5 or GSE5-Like gene is present as more than one copy or homologue (with the same or slightly different sequence) there is at least one mutation in at least one gene. Preferably all genes are mutated.

Grain size and weight are important agronomic traits in crops. We have identified a novel grain size gene (GSE5) that encodes a plasma membrane-associated protein with IQ domains (IQD), which interacts with calmodulin (OsCaM1-1). In rice, loss of GSE5 function causes wide and heavy grains, while overexpression of GSE5 results in narrow and long grains. We have also identified a GSE5-Like protein, that has 72.5% identity with GSE5 and that similarly, a loss of GSE5-Like function increases grain length, grain width and yield. By performing a BLAST search in the databases, we found that GSE5 and GSE5-Like shares significant similarity with its homologs in other crops, such as maize, wheat, sorghum and brachypodium. Our current knowledge of GSE5 and GSE5-Like function suggests that GSE5 and GSE5-Like and its homologs in other crops or plant species can be used to engineer large and heavy seeds in these key crops. We can also use CRISPR/Cas9 technology to knock-out GSE5 or GSE5-Like or its homologs in other crops to increase seed size and weight in these crops. We also can also use RNAi technology to knock-down the expression of GSE5 or GSE5-Like or its homologs in crops to increase seed size and weight in these crops.

In one embodiment, the method comprises introducing at least one mutation into the, preferably endogenous, gene encoding GSE5 or GSE5-Like and/or the GSE5 or GSE5-Like promoter. Preferably said mutation is in the coding region of the GSE5 or GSE5-Like gene. In a further embodiment, at least one mutation or structural alteration may be introduced into the GSE5 or GSE5-Like promoter such that the GSE5 or GSE5-Like gene is either not expressed (i.e. expression is abolished) or expression is reduced, as defined herein. In an alternative embodiment, at least one mutation may be introduced into the GSE5 or GSE5-Like gene such that the altered gene does not express a full-length (i.e. expresses a truncated) GSE5 or GSE5-Like protein or does not express a fully functional GSE5 or GSE5-Like protein. In this manner, the activity of the GSE5 or GSE5-Like polypeptide can be considered to be reduced or abolished as described herein. In any case, the mutation may result in the expression of GSE5 or GSE5-Like with no, significantly reduced or altered biological activity in vivo. Alternatively, GSE5 or GSE5-Like may not be expressed at all.

In another embodiment, the sequence of the GSE5 gene comprises or consists of a nucleic acid sequence as defined in SEQ ID NO: 2 (cDNA) or 32 (genomic) or a functional variant or homologue thereof and encodes a polypeptide as defined in SEQ ID NO: 1 or a functional variant or homologue thereof.

In another embodiment, the sequence of the GSE5-Like gene comprises or consists of a nucleic acid sequence as defined in SEQ ID NO: 55 (cDNA) or 56 (genomic) or a functional variant or homologue thereof and encodes a polypeptide as defined in SEQ ID NO: 57 or a functional variant or homologue thereof.

By “GSE5 promoter” is meant a region extending for at least 6320 bp upstream of the ATG codon of the GSE5 ORF (open reading frame). In one embodiment, the sequence of the GSE5 promoter comprises or consists of a nucleic acid sequence as defined in SEQ ID NO: 28 or a functional variant or homologue thereof. By “GSE5-Like” promoter is meant a region extending at least 2 kb, preferably 6 kb upstream of the GSE5-Like ORF.

In the above embodiments an ‘endogenous’ nucleic acid may refer to the native or natural sequence in the plant genome. In one embodiment, the endogenous sequence of the GSE5 gene comprises SEQ ID NOs: 2 or 32 and encodes an amino acid sequence as defined in SEQ ID NO: 1 or homologs thereof. Similarly, the endogenous sequence of the GSE5-Like gene comprises SEQ ID NOs: 55 or 56 and encodes an amino acid sequence as defined in SEQ ID NO: 57 or homologs thereof. Also included in the scope of this invention are functional variants (as defined herein) and homologs of the above identified sequences. Examples of GSE5 homologs are shown in SEQ ID NOs: 3 to 10. Accordingly, in one embodiment, the homolog encodes a polypeptide selected from SEQ ID NOs: 3, 5, 7 and 9 or the homolog comprises or consists of a nucleic acid sequence selected from SEQ ID NOs: 4, 6, 8 and 10. Examples of GSE5-Like homologs are shown in SEQ ID NOs: 58 to 75. Accordingly, in one embodiment, the homolog encodes a polypeptide selected from SEQ ID NOs: 60, 63, 66, 69, 72 and 75 or the homolog comprises or consists of a nucleic acid sequence selected from SEQ ID NOs: 55, 56, 58, 59, 61, 62, 64, 65, 67, 68, 70, 71, 73 and 74.

The term “functional variant of a nucleic acid sequence” as used herein with reference to any of SEQ ID NOs: 1 to 88 refers to a variant gene sequence or part of the gene sequence which retains the biological function of the full non-variant sequence. A functional variant also comprises a variant of the gene of interest which has sequence alterations that do not affect function, for example in non-conserved residues. Also encompassed is a variant that is substantially identical, i.e. has only some sequence variations, for example in non-conserved residues, compared to the wild type sequences as shown herein and is biologically active. Alterations in a nucleic acid sequence which result in the production of a different amino acid at a given site that do not affect the functional properties of the encoded polypeptide are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

In one embodiment, a functional variant has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid or amino acid sequence.

The term homolog, as used herein, also designates a GSE5 or GSE5-Like promoter or GSE5 or GSE5-Like gene orthologue from other plant species. A homolog may have, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the amino acid represented by any of SEQ ID NO: 1 or 57 or to the nucleic acid sequences as shown by SEQ ID NOs: 2, 32, 55 or 56. In one embodiment, overall sequence identity is at least 37%. In one embodiment, overall sequence identity is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 90%, 91%, 92%, 93%, 94%, 95%, 96%, 97%, 98%, or at least 99%.

Functional variants of GSE5 or GSE5-Like homologs as defined above are also within the scope of the invention.

The “GSE5” or “grain size on chromosome 5” gene encodes a plasma membrane associated protein. This protein is characterised by a IQ calmodulin-binding motif or IQD.

Accordingly, in one embodiment, the GSE5 nucleic acid (coding) sequence encodes a GSE5 protein comprising a IQD domain as defined below, or a variant thereof, wherein the variant has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the IQD as defined herein. In a preferred embodiment, the GSE5 polypeptide is characterised by at least one IQD with at least 75% homology thereto.

In one embodiment, the sequence of the IQD is as follows:

| | |
|---|---|
| [FILV]Qxxx[RK]Gxxx[RK]xx[FILVWY] | (SEQ ID NO: 49) |

Wherein x is any amino acid.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognised that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Non-limiting examples of algorithms that are suitable for determining percent sequence identity and sequence similarity are the BLAST and BLAST 2.0 algorithms.

Suitable homologues can be identified by sequence comparisons and identifications of conserved domains. There are predictors in the art that can be used to identify such sequences. The function of the homologue can be identified as described herein and a skilled person would thus be able to confirm the function, for example when overexpressed in a plant.

Thus, the nucleotide sequences of the invention and described herein can also be used to isolate corresponding sequences from other organisms, particularly other plants, for example crop plants. In this manner, methods such as PCR, hybridization, and the like can be used to identify such sequences based on their sequence homology to the sequences described herein. Topology of the sequences and the characteristic domains structure can also be considered when identifying and isolating homologs. Sequences may be isolated based on their sequence identity to the entire sequence or to fragments thereof. In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen plant. The hybridization probes may be genomic DNA fragments, cDNA fragments, RNA fragments, or other oligonucleotides, and may be labelled with a detectable group, or any other detectable marker. Methods for preparation of probes for hybridization and for construction of cDNA and genomic libraries are generally known in the art and are disclosed in Sambrook, et al., (1989) Molecular Cloning: A Library Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing).

Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than about 24 hours, usually about 4 to 12. Stringent conditions may also be achieved with the addition of destabilizing agents such as formamide.

In a further embodiment, a variant as used herein can comprise a nucleic acid sequence encoding a GSE5 or GSE5-Like polypeptide as defined herein that is capable of hybridising under stringent conditions as defined herein to a nucleic acid sequence as defined in SEQ ID NO: 2 or 32 or 55 or 56.

In one embodiment, there is provided a method of increasing yield in a plant, as described herein, the method comprising reducing or abolishing the expression of at least one nucleic acid encoding a GSE5 or GSE5-Like polypeptide, as described herein, wherein the method comprises introducing at least one mutation into at least

GSE5 or GSE5-Like gene and/or promoter, wherein the GSE5 or GSE5-Like gene comprises or consists of
a. a nucleic acid sequence encoding a polypeptide as defined in one of SEQ ID NO:1, 3, 5, 7, 9, 57, 60, 63, 66, 69, 73 and 75; or
b. a nucleic acid sequence as defined in one of SEQ ID NO: 2, 32, 4, 6, 8, 10, 55, 56, 58, 59, 61, 62, 64, 65, 67, 68, 70, 71, 73 and 74; or
c. a nucleic acid sequence with at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to either (a) or (b); or
d. a nucleic acid sequence encoding a GSE5 or GSE5-Like polypeptide as defined herein that is capable of hybridising under stringent conditions as defined herein to the nucleic acid sequence of any of (a) to (c).
   and wherein the GSE5 promoter comprises or consists of
e. a nucleic acid sequence as defined in one of SEQ ID NOs 28
f. a nucleic acid sequence with at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to (e); or
g. a nucleic acid sequence capable of hybridising under stringent conditions as defined herein to the nucleic acid sequence of any of (e) to (f).

In a preferred embodiment, the mutation that is introduced into the endogenous GSE5 or GSE5-Like gene or promoter thereof to silence, reduce, or inhibit the biological activity and/or expression levels of the GSE5 or GSE5-Like gene or protein can be selected from the following mutation types
1. a “missense mutation”, which is a change in the nucleic acid sequence that results in the substitution of an amino acid for another amino acid;
2. a “nonsense mutation” or “STOP codon mutation”, which is a change in the nucleic acid sequence that results in the introduction of a premature STOP codon and, thus, the termination of translation (resulting in a truncated protein); plant genes contain the translation stop codons “TGA” (UGA in RNA), “TAA” (UAA in RNA) and “TAG” (UAG in RNA); thus any nucleotide substitution, insertion, deletion which results in one of these codons to be in the mature mRNA being translated (in the reading frame) will terminate translation.
3. an “insertion mutation” of one or more amino acids, due to one or more codons having been added in the coding sequence of the nucleic acid;
4. a “deletion mutation” of one or more amino acids, due to one or more codons having been deleted in the coding sequence of the nucleic acid;
5. a “frameshift mutation”, resulting in the nucleic acid sequence being translated in a different frame downstream of the mutation. A frameshift mutation can have various causes, such as the insertion, deletion or duplication of one or more nucleotides.
6. a “splice site” mutation, which is a mutation that results in the insertion, deletion or substitution of a nucleotide at the site of splicing.

As used herein, an “insertion” may refer to the insertion of at least one nucleotide. In one embodiment said insertion may be between 20 and 500 base pairs, more preferably between 300 and 400 base pairs.

As used herein, a “deletion” may refer to the deletion of at least one nucleotide. In one embodiment, said deletion may be between 1 and 1500 base pairs, more preferably between 900 and 1300 base pairs.

In general, the skilled person will understand that at least one mutation as defined above and which leads to the insertion, deletion or substitution of at least one nucleic acid or amino acid compared to the wild-type GSE5 promoter or GSE5 or GSE5-Like nucleic acid or protein sequence can affect the biological activity of the GSE5 or GSE5-Like protein.

In one embodiment, the mutation is introduced into the IQ domain of GSE5. Preferably, said mutation is a loss of function mutation such as a premature stop codon, or an amino acid change in a highly conserved region that is predicted to be important for protein structure.

In another embodiment, the mutation is introduced into the GSE5 or GSE5-Like promoter and is at least the deletion and/or insertion of at least one nucleic acid. In one embodiment, a sequence comprising or consisting of SEQ ID NO: 29 or 30 or a variant thereof is deleted. In a further or alternative embodiment a sequence comprising or consisting of SEQ ID NO: 31 or a variant thereof is inserted. Other major changes such as deletions that remove functional regions of the promoter are also included as these will reduce the expression of GSE5.

In one embodiment a mutation may be introduced into the GSE5 or GSE5-Like promoter and at least one mutation is introduced into the GSE5 or GSE5-Like gene.

In one embodiment, the mutation is introduced using mutagenesis or targeted genome editing. That is, in one embodiment, the invention relates to a method and plant that has been generated by genetic engineering methods as described above, and does not encompass naturally occurring varieties.

Targeted genome modification or targeted genome editing is a genome engineering technique that uses targeted DNA double-strand breaks (DSBs) to stimulate genome editing through homologous recombination (HR)-mediated recombination events. To achieve effective genome editing via introduction of site-specific DNA DSBs, four major classes of customisable DNA binding proteins can be used: meganucleases derived from microbial mobile genetic elements, ZF nucleases based on eukaryotic transcription factors, transcription activator-like effectors (TALEs) from Xanthomonas bacteria, and the RNA-guided DNA endonuclease Cas9 from the type II bacterial adaptive immune system CRISPR (clustered regularly interspaced short palindromic repeats). Meganuclease, ZF, and TALE proteins all recognize specific DNA sequences through protein-DNA interactions. Although meganucleases integrate nuclease and DNA-binding domains, ZF and TALE proteins consist of individual modules targeting 3 or 1 nucleotides (nt) of DNA, respectively. ZFs and TALEs can be assembled in desired combinations and attached to the nuclease domain of Fokl to direct nucleolytic activity toward specific genomic loci.

Upon delivery into host cells via the bacterial type III secretion system, TAL effectors enter the nucleus, bind to effector-specific sequences in host gene promoters and activate transcription. Their targeting specificity is determined by a central domain of tandem, 33-35 amino acid repeats. This is followed by a single truncated repeat of 20 amino acids. The majority of naturally occurring TAL effectors examined have between 12 and 27 full repeats.

These repeats only differ from each other by two adjacent amino acids, their repeat-variable di-residue (RVD). The RVD that determines which single nucleotide the TAL effector will recognize: one RVD corresponds to one nucleotide, with the four most common RVDs each preferentially associating with one of the four bases. Naturally occurring recognition sites are uniformly preceded by a T that is required for TAL effector activity. TAL effectors can be fused to the catalytic domain of the Fokl nuclease to create a TAL effector nuclease (TALEN) which makes targeted DNA double-strand breaks (DSBs) in vivo for genome editing. The use of this technology in genome editing is well described in the art, for example in U.S. Pat. Nos. 8,440,431, 8,440,432 and 8,450,471. Cermak T et al. describes a set of customized plasmids that can be used with the Golden Gate cloning method to assemble multiple DNA fragments. As described therein, the Golden Gate method uses Type IIS restriction endonucleases, which cleave outside their recognition sites to create unique 4 bp overhangs. Cloning is expedited by digesting and ligating in the same reaction mixture because correct assembly eliminates the enzyme recognition site. Assembly of a custom TALEN or TAL effector construct and involves two steps: (i) assembly of repeat modules into intermediary arrays of 1-10 repeats and (ii) joining of the intermediary arrays into a backbone to make the final construct. Accordingly, using techniques known in the art it is possible to design a TAL effector that targets a GSE5 or GSE5-Like gene or promoter sequence as described herein.

Another genome editing method that can be used according to the various aspects of the invention is CRISPR. The use of this technology in genome editing is well described in the art, for example in U.S. Pat. No. 8,697,359 and references cited herein. In short, CRISPR is a microbial nuclease system involved in defense against invading phages and plasmids. CRISPR loci in microbial hosts contain a combination of CRISPR-associated (Cas) genes as well as non-coding RNA elements capable of programming the specificity of the CRISPR-mediated nucleic acid cleavage (sgRNA). Three types (I-III) of CRISPR systems have been identified across a wide range of bacterial hosts. One key feature of each CRISPR locus is the presence of an array of repetitive sequences (direct repeats) interspaced by short stretches of non-repetitive sequences (spacers). The non-coding CRISPR array is transcribed and cleaved within direct repeats into short crRNAs containing individual spacer sequences, which direct Cas nucleases to the target site (protospacer). The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer.

One major advantage of the CRISPR-Cas9 system, as compared to conventional gene targeting and other programmable endonucleases is the ease of multiplexing, where multiple genes can be mutated simultaneously simply by using multiple sgRNAs each targeting a different gene. In addition, where two sgRNAs are used flanking a genomic region, the intervening section can be deleted or inverted (Wiles et al., 2015).

Cas9 is thus the hallmark protein of the type II CRISPR-Cas system, and is a large monomeric DNA nuclease guided to a DNA target sequence adjacent to the PAM (protospacer adjacent motif) sequence motif by a complex of two noncoding RNAs: CRISPR RNA (crRNA) and trans-activating crRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with an sgRNA can introduce site-specific double strand breaks (DSBs) into genomic DNA of live cells from various organisms. For applications in eukaryotic organisms, codon optimized versions of Cas9, which is originally from the bacterium Streptococcus pyogenes, have been used.

The single guide RNA (sgRNA) is the second component of the CRISPR/Cas system that forms a complex with the Cas9 nuclease. sgRNA is a synthetic RNA chimera created by fusing crRNA with tracrRNA. The sgRNA guide sequence located at its 5′ end confers DNA target specificity. Therefore, by modifying the guide sequence, it is possible to create sgRNAs with different target specificities. The canonical length of the guide sequence is 20 bp. In plants, sgRNAs have been expressed using plant RNA polymerase III promoters, such as U6 and U3. Accordingly, using techniques known in the art it is possible to design sgRNA molecules that target a GSE5 or GSE5-Like gene or promoter sequence as described herein. In one embodiment, the sgRNA molecules target a sequence selected from SEQ ID No: 15 to 20, 48, 51, 76 or 79 to 84 or a variant thereof as defined herein. In a further embodiment, the sgRNA molecules comprises a protospacer sequence selected from SEQ ID NO: 21 to 26 and 52 and 77 or a variant thereof, as defined herein. In a further embodiment, the sgRNA nucleic acid sequence comprises a sequence comprising or consisting of SEQ ID NO: 78 or 89 or a variant thereof, as defined herein.

Cas9 expression plasmids for use in the methods of the invention can be constructed as described in the art.

In one embodiment, the method uses the sgRNA constructs defined in detail below to introduce a targeted mutation into a GSE5 or GSE5-Like gene and/or promoter.

Alternatively, more conventional mutagenesis methods can be used to introduce at least one mutation into a GSE5 or GSE5-Like gene or GSE5 or GSE5-Like promoter sequence. These methods include both physical and chemical mutagenesis. A skilled person will know further approaches can be used to generate such mutants, and methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein.

In one embodiment, insertional mutagenesis is used, for example using T-DNA mutagenesis (which inserts pieces of the T-DNA from the Agrobacterium tumefaciens T-Plasmid into DNA causing either loss of gene function or gain of gene function mutations), site-directed nucleases (SDNs) or transposons as a mutagen. Insertional mutagenesis is an alternative means of disrupting gene function and is based on the insertion of foreign DNA into the gene of interest (see Krysan et al, The Plant Cell, Vol. 11, 2283-2290, December 1999). Accordingly, in one embodiment, T-DNA is used as an insertional mutagen to disrupt the GSE5 or GSE5-Like gene or GSE5 or GSE5-Like promoter expression. An example of using T-DNA mutagenesis to disrupt the Arabidopsis GSE5 gene is described in Downes et al. 2003. T-DNA not only disrupts the expression of the gene into which it is inserted, but also acts as a marker for subsequent identification of the mutation. Since the sequence of the inserted element is known, the gene in which the insertion has occurred can be recovered, using various cloning or PCR-based strategies. The insertion of a piece of T-DNA in the order of 5 to 25 kb in length generally produces a disruption of gene function. If a large enough population of T-DNA transformed lines is generated, there are reasonably good chances of finding a transgenic plant carrying a T-DNA insert within any gene of interest. Transformation of spores with T-DNA is achieved by an Agrobacterium-mediated method which involves exposing plant cells and tissues to a suspension of Agrobacterium cells.

The details of this method are well known to a skilled person. In short, plant transformation by Agrobacterium results in the integration into the nuclear genome of a sequence called T-DNA, which is carried on a bacterial plasmid. The use of T-DNA transformation leads to stable single insertions. Further mutant analysis of the resultant transformed lines is straightforward and each individual insertion line can be rapidly characterized by direct sequencing and analysis of DNA flanking the insertion. Gene expression in the mutant is compared to expression of the GSE5 or GSE5-Like nucleic acid sequence in a wild type plant and phenotypic analysis is also carried out.

In another embodiment, mutagenesis is physical mutagenesis, such as application of ultraviolet radiation, X-rays, gamma rays, fast or thermal neutrons or protons. The targeted population can then be screened to identify a GSE5 or GSE5-Like loss of function mutant.

In another embodiment of the various aspects of the invention, the method comprises mutagenizing a plant population with a mutagen. The mutagen may be a fast neutron irradiation or a chemical mutagen, for example selected from the following non-limiting list: ethyl methanesulfonate (EMS), methylmethane sulfonate (MMS), N-ethyl-N-nitrosurea (ENU), triethylmelamine (1′EM), N-methyl-N-nitrosourea (MNU), procarbazine, chlorambucil, cyclophosphamide, diethyl sulfate, acrylamide monomer, melphalan, nitrogen mustard, vincristine, dimethylnitosamine, N-methyl-N′-nitro-Nitrosoguanidine (MNNG), nitrosoguanidine, 2-aminopurine, 7,12 dimethyl-benz(a)anthracene (DMBA), ethylene oxide, hexamethylphosphoramide, bisulfan, diepoxyalkanes (diepoxyoctane (DEO), diepoxybutane (BEB), and the like), 2-methoxy-6-chloro-9 [3-(ethyl-2-chloroethyl)aminopropylamino]acridine dihydrochloride (ICR-170) or formaldehyde. Again, the targeted population can then be screened to identify a GSE5 or GSE5-Like gene or promoter mutant.

In another embodiment, the method used to create and analyse mutations is targeting induced local lesions in genomes (TILLING), reviewed in Henikoff et al, 2004. In this method, seeds are mutagenised with a chemical mutagen, for example EMS. The resulting M1 plants are self-fertilised and the M2 generation of individuals is used to prepare DNA samples for mutational screening. DNA samples are pooled and arrayed on microtiter plates and subjected to gene specific PCR. The PCR amplification products may be screened for mutations in the GSE5 or GSE5-Like target gene using any method that identifies heteroduplexes between wild type and mutant genes. For example, but not limited to, denaturing high pressure liquid chromatography (dHPLC), constant denaturant capillary electrophoresis (CDCE), temperature gradient capillary electrophoresis (TGCE), or by fragmentation using chemical cleavage. Preferably the PCR amplification products are incubated with an endonuclease that preferentially cleaves mismatches in heteroduplexes between wild type and mutant sequences. Cleavage products are electrophoresed using an automated sequencing gel apparatus, and gel images are analyzed with the aid of a standard commercial image-processing program. Any primer specific to the GSE5 or GSE5-Like nucleic acid sequence may be utilized to amplify the GSE5 or GSE5-Like nucleic acid sequence within the pooled DNA sample. Preferably, the primer is designed to amplify the regions of the GSE5 or GSE5-Like gene where useful mutations are most likely to arise, specifically in the areas of the GSE5 or GSE5-Like gene that are highly conserved and/or confer activity as explained elsewhere. To facilitate detection of PCR products on a gel, the PCR primer may be labelled using any conventional labelling method. In an alternative embodiment, the method used to create and analyse mutations is EcoTILLING. EcoTILLING is molecular technique that is similar to TILLING, except that its objective is to uncover natural variation in a given population as opposed to induced mutations. The first publication of the EcoTILLING method was described in Comai et al. 2004.

Rapid high-throughput screening procedures thus allow the analysis of amplification products for identifying a mutation conferring the reduction or inactivation of the expression of the GSE5 or GSE5-Like gene as compared to a corresponding non-mutagenised wild type plant. Once a mutation is identified in a gene of interest, the seeds of the M2 plant carrying that mutation are grown into adult M3 plants and screened for the phenotypic characteristics associated with the target gene GSE5 or GSE5-Like. Loss of and reduced function mutants with increased seed size compared to a control can thus be identified.

Plants obtained or obtainable by such method which carry a functional mutation in the endogenous GSE5 or GSE5-Like gene or promoter locus are also within the scope of the invention

In an alternative embodiment, the expression of the GSE5 or GSE5-Like gene may be reduced at either the level of transcription or translation. For example, expression of a GSE5 or GSE5-Like nucleic acid or GSE5 or GSE5-Like promoter sequence, as defined herein, can be reduced or silenced using a number of gene silencing methods known to the skilled person, such as, but not limited to, the use of small interfering nucleic acids (siNA) against GSE5 or GSE5-Like. “Gene silencing” is a term generally used to refer to suppression of expression of a gene via sequence-specific interactions that are mediated by RNA molecules. The degree of reduction may be so as to totally abolish production of the encoded gene product, but more usually the abolition of expression is partial, with some degree of expression remaining. The term should not therefore be taken to require complete “silencing” of expression.

In one embodiment, the siNA may include, short interfering RNA (siRNA), double-stranded RNA (dsRNA), micro-RNA (miRNA), antagomirs and short hairpin RNA (shRNA) capable of mediating RNA interference.

The inhibition of expression and/or activity can be measured by determining the presence and/or amount of GSE5 or GSE5-Like transcript using techniques well known to the skilled person (such as Northern Blotting, RT-PCR and so on).

Transgenes may be used to suppress endogenous plant genes. This was discovered originally when chalcone synthase transgenes in petunia caused suppression of the endogenous chalcone synthase genes and indicated by easily visible pigmentation changes. Subsequently it has been described how many, if not all plant genes can be “silenced” by transgenes. Gene silencing requires sequence similarity between the transgene and the gene that becomes silenced. This sequence homology may involve promoter regions or coding regions of the silenced target gene. When coding regions are involved, the transgene able to cause gene silencing may have been constructed with a promoter that would transcribe either the sense or the antisense orientation of the coding sequence RNA. It is likely that the various examples of gene silencing involve different mechanisms that are not well understood. In different examples there may be transcriptional or post-transcriptional gene silencing and both may be used according to the methods of the invention.

The mechanisms of gene silencing and their application in genetic engineering, which were first discovered in plants in the early 1990s and then shown in Caenorhabditis elegans are extensively described in the literature.

RNA-mediated gene suppression or RNA silencing according to the methods of the invention includes co-suppression wherein over-expression of the target sense RNA or mRNA, that is the GSE5 or GSE5-Like sense RNA or mRNA, leads to a reduction in the level of expression of the genes concerned. RNAs of the transgene and homologous endogenous gene are co-ordinately suppressed. Other techniques used in the methods of the invention include antisense RNA to reduce transcript levels of the endogenous target gene in a plant. In this method, RNA silencing does not affect the transcription of a gene locus, but only causes sequence-specific degradation of target mRNAs. An “antisense” nucleic acid sequence comprises a nucleotide sequence that is complementary to a “sense” nucleic acid sequence encoding a GSE5 or GSE5-Like protein, or a part of the protein, i.e. complementary to the coding strand of a double-stranded cDNA molecule or complementary to an mRNA transcript sequence. The antisense nucleic acid sequence is preferably complementary to the endogenous GSE5 or GSE5-Like gene to be silenced. The complementarity may be located in the “coding region” and/or in the “non-coding region” of a gene. The term “coding region” refers to a region of the nucleotide sequence comprising codons that are translated into amino acid residues. The term “non-coding region” refers to 5′ and 3′ sequences that flank the coding region that are transcribed but not translated into amino acids (also referred to as 5′ and 3′ untranslated regions).

Antisense nucleic acid sequences can be designed according to the rules of Watson and Crick base pairing. The antisense nucleic acid sequence may be complementary to the entire GSE5 or GSE5-Like nucleic acid sequence as defined herein, but may also be an oligonucleotide that is antisense to only a part of the nucleic acid sequence (including the mRNA 5′ and 3′ UTR). For example, the antisense oligonucleotide sequence may be complementary to the region surrounding the translation start site of an mRNA transcript encoding a polypeptide. The length of a suitable antisense oligonucleotide sequence is known in the art and may start from about 50, 45, 40, 35, 30, 25, 20, 15 or 10 nucleotides in length or less. An antisense nucleic acid sequence according to the invention may be constructed using chemical synthesis and enzymatic ligation reactions using methods known in the art. For example, an antisense nucleic acid sequence (e.g., an antisense oligonucleotide sequence) may be chemically synthesized using naturally occurring nucleotides or variously modified nucleotides designed to increase the biological stability of the molecules or to increase the physical stability of the duplex formed between the antisense and sense nucleic acid sequences, e.g., phosphorothioate derivatives and acridine-substituted nucleotides may be used. Examples of modified nucleotides that may be used to generate the antisense nucleic acid sequences are well known in the art. The antisense nucleic acid sequence can be produced biologically using an expression vector into which a nucleic acid sequence has been subcloned in an antisense orientation (i.e., RNA transcribed from the inserted nucleic acid will be of an antisense orientation to a target nucleic acid of interest). Preferably, production of antisense nucleic acid sequences in plants occurs by means of a stably integrated nucleic acid construct comprising a promoter, an operably linked antisense oligonucleotide, and a terminator.

The nucleic acid molecules used for silencing in the methods of the invention hybridize with or bind to mRNA transcripts and/or insert into genomic DNA encoding a polypeptide to thereby inhibit expression of the protein, e.g., by inhibiting transcription and/or translation. The hybridization can be by conventional nucleotide complementarity to form a stable duplex, or, for example, in the case of an antisense nucleic acid sequence which binds to DNA duplexes, through specific interactions in the major groove of the double helix. Antisense nucleic acid sequences may be introduced into a plant by transformation or direct injection at a specific tissue site. Alternatively, antisense nucleic acid sequences can be modified to target selected cells and then administered systemically. For example, for systemic administration, antisense nucleic acid sequences can be modified such that they specifically bind to receptors or antigens expressed on a selected cell surface, e.g., by linking the antisense nucleic acid sequence to peptides or antibodies which bind to cell surface receptors or antigens. The antisense nucleic acid sequences can also be delivered to cells using vectors.

RNA interference (RNAi) is another post-transcriptional gene-silencing phenomenon which may be used according to the methods of the invention. This is induced by double-stranded RNA in which mRNA that is homologous to the dsRNA is specifically degraded. It refers to the process of sequence-specific post-transcriptional gene silencing mediated by short interfering RNAs (siRNA). The process of RNAi begins when the enzyme, DICER, encounters dsRNA and chops it into pieces called small-interfering RNAs (siRNA). This enzyme belongs to the RNase III nuclease family. A complex of proteins gathers up these RNA remains and uses their code as a guide to search out and destroy any RNAs in the cell with a matching sequence, such as target mRNA.

Artificial and/or natural microRNAs (miRNAs) may be used to knock out gene expression and/or mRNA translation. MicroRNAs (miRNAs) miRNAs are typically single stranded small RNAs typically 19-24 nucleotides long. Most plant miRNAs have perfect or near-perfect complementarity with their target sequences. However, there are natural targets with up to five mismatches. They are processed from longer non-coding RNAs with characteristic fold-back structures by double-strand specific RNases of the Dicer family. Upon processing, they are incorporated in the RNA-induced silencing complex (RISC) by binding to its main component, an Argonaute protein.

miRNAs serve as the specificity components of RISC, since they base-pair to target nucleic acids, mostly mRNAs, in the cytoplasm. Subsequent regulatory events include target mRNA cleavage and destruction and/or translational inhibition. Effects of miRNA overexpression are thus often reflected in decreased mRNA levels of target genes. Artificial microRNA (amiRNA) technology has been applied in Arabidopsis thaliana and other plants to efficiently silence target genes of interest. The design principles for amiRNAs have been generalized and integrated into a Web-based tool (http://wmd.weiqelworld.orq).

Thus, according to the various aspects of the invention a plant may be transformed to introduce a RNAi, shRNA, snRNA, dsRNA, siRNA, miRNA, ta-siRNA, amiRNA or cosuppression molecule that has been designed to target the expression of an GSE5 nucleic acid sequence and selectively decreases or inhibits the expression of the gene or stability of its transcript. Preferably, the RNAi, snRNA, dsRNA, shRNA siRNA, miRNA, amiRNA, ta-siRNA or cosuppression molecule used according to the various aspects of the invention comprises a fragment of at least 17 nt, preferably 22 to 26 nt and can be designed on the basis of the information shown in any of SEQ ID NOs:1 to 14 or 55 to 75. Guidelines for designing effective siRNAs are known to the skilled person. Briefly, a short fragment of the target gene sequence (e.g., 19-40 nucleotides in length) is chosen as the target sequence of the siRNA of the invention. The short fragment of target gene sequence is a fragment of the target gene mRNA. In preferred embodiments, the criteria for choosing a sequence fragment from the target gene mRNA to be a candidate siRNA molecule include 1) a sequence from the target gene mRNA that is at least 50-100 nucleotides from the 5′ or 3′ end of the native mRNA molecule, 2) a sequence from the target gene mRNA that has a G/C content of between 30% and 70%, most preferably around 50%, 3) a sequence from the target gene mRNA that does not contain repetitive sequences (e.g., AAA, CCC, GGG, TTT, AAAA, CCCC, GGGG, TTTT), 4) a sequence from the target gene mRNA that is accessible in the mRNA, 5) a sequence from the target gene mRNA that is unique to the target gene, 6) avoids regions within 75 bases of a start codon. The sequence fragment from the target gene mRNA may meet one or more of the criteria identified above. The selected gene is introduced as a nucleotide sequence in a prediction program that takes into account all the variables described above for the design of optimal oligonucleotides. This program scans any mRNA nucleotide sequence for regions susceptible to be targeted by siRNAs. The output of this analysis is a score of possible siRNA oligonucleotides. The highest scores are used to design double stranded RNA oligonucleotides that are typically made by chemical synthesis. In addition to siRNA which is complementary to the mRNA target region, degenerate siRNA sequences may be used to target homologous regions. siRNAs according to the invention can be synthesized by any method known in the art. RNAs are preferably chemically synthesized using appropriately protected ribonucleoside phosphoramidites and a conventional DNA/RNA synthesizer. Additionally, siRNAs can be obtained from commercial RNA oligonucleotide synthesis suppliers.

siRNA molecules according to the aspects of the invention may be double stranded. In one embodiment, double stranded siRNA molecules comprise blunt ends. In another embodiment, double stranded siRNA molecules comprise overhanging nucleotides (e.g., 1-5 nucleotide overhangs, preferably 2 nucleotide overhangs). In some embodiments, the siRNA is a short hairpin RNA (shRNA); and the two strands of the siRNA molecule may be connected by a linker region (e.g., a nucleotide linker or a non-nucleotide linker). The siRNAs of the invention may contain one or more modified nucleotides and/or non-phosphodiester linkages. Chemical modifications well known in the art are capable of increasing stability, availability, and/or cell uptake of the siRNA. The skilled person will be aware of other types of chemical modification which may be incorporated into RNA molecules.

In one embodiment, recombinant DNA constructs as described in U.S. Pat. No. 6,635,805, incorporated herein by reference, may be used.

The silencing RNA molecule is introduced into the plant using conventional methods, for example a vector and Agrobacterium-mediated transformation. Stably transformed plants are generated and expression of the GSE5 or GSE5-Like gene compared to a wild type control plant is analysed.

Silencing of the GSE5 or GSE5-Like nucleic acid sequence may also be achieved using virus-induced gene silencing.

Thus, in one embodiment of the invention, the plant expresses a nucleic acid construct comprising a RNAi, shRNA snRNA, dsRNA, siRNA, miRNA, ta-siRNA, amiRNA or co-suppression molecule that targets the GSE5 or GSE5-Like nucleic acid sequence as described herein and reduces expression of the endogenous GSE5 or GSE5-Like nucleic acid sequence. A gene is targeted when, for example, the RNAi, snRNA, dsRNA, siRNA, shRNA miRNA, ta-siRNA, amiRNA or cosuppression molecule selectively decreases or inhibits the expression of the gene compared to a control plant. Alternatively, a RNAi, snRNA, dsRNA, siRNA, miRNA, ta-siRNA, amiRNA or cosuppression molecule targets a GSE5 or GSE5-Like nucleic acid sequence when the RNAi, shRNA snRNA, dsRNA, siRNA, miRNA, ta-siRNA, amiRNA or cosuppression molecule hybridises under stringent conditions to the gene transcript.

A further approach to gene silencing is by targeting nucleic acid sequences complementary to the regulatory region of the gene (e.g., the promoter and/or enhancers) of GSE5 or GSE5-Like to form triple helical structures that prevent transcription of the gene in target cells. Other methods, such as the use of antibodies directed to an endogenous polypeptide for inhibiting its function in planta, or interference in the signalling pathway in which a polypeptide is involved, will be well known to the skilled man. In particular, it can be envisaged that manmade molecules may be useful for inhibiting the biological function of a target polypeptide, or for interfering with the signalling pathway in which the target polypeptide is involved.

In one embodiment, the suppressor nucleic acids may be anti-sense suppressors of expression of the GSE5 or GSE5-Like polypeptides. In using anti-sense sequences to down-regulate gene expression, a nucleotide sequence is placed under the control of a promoter in a “reverse orientation” such that transcription yields RNA which is complementary to normal mRNA transcribed from the “sense” strand of the target gene.

An anti-sense suppressor nucleic acid may comprise an anti-sense sequence of at least 10 nucleotides from the target nucleotide sequence. It may be preferable that there is complete sequence identity in the sequence used for down-regulation of expression of a target sequence, and the target sequence, although total complementarity or similarity of sequence is not essential. One or more nucleotides may differ in the sequence used from the target gene. Thus, a sequence employed in a down-regulation of gene expression in accordance with the present invention may be a wild-type sequence (e.g. gene) selected from those available, or a variant of such a sequence.

The sequence need not include an open reading frame or specify an RNA that would be translatable. It may be preferred for there to be sufficient homology for the respective anti-sense and sense RNA molecules to hybridise. There may be down regulation of gene expression even where there is about 5%, 10%, 15% or 20% or more mismatch between the sequence used and the target gene. Effectively, the homology should be sufficient for the down-regulation of gene expression to take place.

Suppressor nucleic acids may be operably linked to tissue-specific or inducible promoters. For example, integument and seed specific promoters can be used to specifically down-regulate a GSE5 or GSE5-Like nucleic acid in developing ovules and seeds to increase final seed size.

Nucleic acid which suppresses expression of a GSE5 or GSE5-Like polypeptide as described herein may be operably linked to a heterologous regulatory-sequence, such as a promoter, for example a constitutive, inducible, tissue-specific or developmental specific promoter. The construct or vector may be transformed into plant cells and expressed as described herein. Plant cells comprising such vectors are also within the scope of the invention.

In another aspect, the invention relates to a silencing construct obtainable or obtained by a method as described herein and to a plant cell comprising such construct.

Thus, aspects of the invention involve targeted mutagenesis methods, specifically genome editing, and in a preferred embodiment exclude embodiments that are solely based on generating plants by traditional breeding methods.

In a further embodiment, the method may comprise reducing and/or abolishing the activity of GSE5 or GSE5-Like. In one example this may comprise reducing GSE5's ability to interact with calmodulin by mutating the IQ domain as described herein.

In another aspect, the invention extends to a plant obtained or obtainable by a method as described herein.

In a further aspect of the invention, there is provided a method of increasing cell proliferation in the spiklet hull of a plant, preferably in the grain-width direction, the method comprising reducing or abolishing the expression of at least one nucleic acid encoding a grain size on chromosome 5 (referred to herein as GSE5) or GSE5-Like polypeptide and/or reducing the activity of a GSE5 or GSE5-Like polypeptide in said plant. The terms “increase”, “improve” or “enhance” as used herein are interchangeable. In one embodiment, cell proliferation is increased by at least 2%, 3%, 4%, 5%, 6%, 7%, 8%, 9%, 10% 11%, 12%, 13%, 14%, 15%, 16%, 17%, 18%, 19%, 20%, 30%, 40% or 50% in comparison to a control plant.

### Genetically Altered or Modified Plants and Methods of Producing Such Plants

In another aspect of the invention there is provided a genetically altered plant, part thereof or plant cell characterised in that the plant does not express GSE5 or GSE5-Like, has reduced levels of GSE5 or GSE5-Like expression, does not express a functional GSE5 or GSE5-Like protein or expresses a GSE5 or GSE5-Like protein with reduced function and/or activity. For example, the plant is a reduction (knock down) or loss of function (knock out) mutant wherein the function of the GSE5 or GSE5-Like nucleic acid sequence is reduced or lost compared to a wild type control plant. To this end, a mutation is introduced into either the GSE5 or GSE5-Like gene sequence or the corresponding promoter sequence which disrupts the transcription of the gene. Therefore, preferably said plant comprises at least one mutation in the promoter and/or gene for GSE5 and/or GSE5-Like. In one embodiment the plant may comprise a mutation in both the promoter and gene for GSE5 or GSE5-Like.

In a further aspect of the invention, there is provided a plant, part thereof or plant cell characterised by an increased seed yield compared to a wild-type or control pant, wherein preferably, the plant comprises at least one mutation in the GSE5 or GSE5-Like gene and/or its promoter. Preferably said increase in seed yield comprises an increase in at least one of seed weight, seed width and TKW.

The plant may be produced by introducing a mutation, preferably a deletion, insertion or substitution into the GSE5 or GSE5-Like gene and/or promoter sequence by any of the above described methods. Preferably said mutation is introduced into a least one plant cell and a plant regenerated from the at least one mutated plant cell.

Alternatively, the plant or plant cell may comprise a nucleic acid construct expressing an RNAi molecule targeting the GSE or GSE5-Like gene as described herein. In one embodiment, said construct is stably incorporated into the plant genome. These techniques also include gene targeting using vectors that target the gene of interest and which allow integration of a transgene at a specific site. The targeting construct is engineered to recombine with the target gene, which is accomplished by incorporating sequences from the gene itself into the construct. Recombination then occurs in the region of that sequence within the gene, resulting in the insertion of a foreign sequence to disrupt the gene. With its sequence interrupted, the altered gene will be translated into a nonfunctional protein, if it is translated at all.

In another aspect of the invention there is provided a method for producing a genetically altered plant as described herein. In one embodiment, the method comprises introducing at least one mutation into the GSE5 or GSE5-Like gene and/or GSE5 or GSE5-Like promoter of preferably at least one plant cell using any mutagenesis technique described herein. Preferably said method further comprising regenerating a plant from the mutated plant cell.

The method may further comprise selecting one or more mutated plants, preferably for further propagation. Preferably said selected plants comprise at least one mutation in the GSE5 or GSE5-Like gene and/or promoter sequence. Preferably said plants are characterised by abolished or a reduced level of GSE5 or GSE5-Like expression and/or a reduced level of GSE5 or GSE5-Like polypeptide activity. Expression and/or activity levels of GSE5 or GSE5-Like can be measured by any standard technique known to the skilled person. In one embodiment GSE5 binding to calmodulin could be measured. A reduction is as described herein.

The selected plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

In a further aspect of the invention there is provided a plant obtained or obtainable by the above described methods.

For the purposes of the invention, a “genetically altered plant” or “mutant plant” is a plant that has been genetically altered compared to the naturally occurring wild type (WT) plant. In one embodiment, a mutant plant is a plant that has been altered compared to the naturally occurring wild type (WT) plant using a mutagenesis method, such as any of the mutagenesis methods described herein. In one embodiment, the mutagenesis method is targeted genome modification or genome editing. In one embodiment, the plant genome has been altered compared to wild type sequences using a mutagenesis method. Such plants have an altered phenotype as described herein, such as an increased seed yield. Therefore, in this example, increased seed yield is conferred by the presence of an altered plant genome, for example, a mutated endogenous GSE5 or GSE5-Like gene or GSE5 or GSE5-Like promoter sequence. In one embodiment, the endogenous promoter or gene sequence is specifically targeted using targeted genome modification and the presence of a mutated gene or promoter sequence is not conferred by the presence of transgenes expressed in the plant. In other words, the genetically altered plant can be described as transgene-free.

A plant according to the various aspects of the invention, including the transgenic plants, methods and uses described herein may be a monocot or a dicot plant. Preferably, the plant is a crop plant. By crop plant is meant any plant which is grown on a commercial scale for human or animal consumption or use. In a preferred embodiment, the plant is a cereal. In another embodiment the plant is Arabidopsis or Medicago truncatula.

In a most preferred embodiment, the plant is selected from rice, wheat, maize, soybean and sorghum. In a most preferred embodiment the plant is rice, preferably the japonica or indica varieties.

The term “plant” as used herein encompasses whole plants, ancestors and progeny of the plants and plant parts, including seeds, fruit, shoots, stems, leaves, roots (including tubers), flowers, tissues and organs, wherein each of the aforementioned comprise the nucleic acid construct as described herein. The term “plant” also encompasses plant cells, suspension cultures, callus tissue, embryos, meristematic regions, gametophytes, sporophytes, pollen and microspores, again wherein each of the aforementioned comprises the nucleic acid construct as described herein.

The invention also extends to harvestable parts of a plant of the invention as described herein, but not limited to seeds, leaves, fruits, flowers, stems, roots, rhizomes, tubers and bulbs. The aspects of the invention also extend to products derived, preferably directly derived, from a harvestable part of such a plant, such as dry pellets or powders, oil, fat and fatty acids, starch or proteins. Another product that may derived from the harvestable parts of the plant of the invention is biodiesel. The invention also relates to food products and food supplements comprising the plant of the invention or parts thereof. In one embodiment, the food products may be animal feed. In another aspect of the invention, there is provided a product derived from a plant as described herein or from a part thereof.

In a most preferred embodiment, the plant part or harvestable product is a seed or grain. Therefore, in a further aspect of the invention, there is provided a seed produced from a genetically altered plant as described herein. In an alternative embodiment, the plant part is pollen, a propagule or progeny of the genetically altered plant described herein. Accordingly, in a further aspect of the invention there is provided pollen, a propagule or progeny of the genetically altered plant as described herein.

A control plant as used herein according to all of the aspects of the invention is a plant which has not been modified according to the methods of the invention. Accordingly, in one embodiment, the control plant does not have reduced expression of a GSE5 or GSE5-Like nucleic acid and/or reduced activity of a GSE5 or GSE5-Like polypeptide. In an alternative embodiment, the plant been genetically modified, as described above. In one embodiment, the control plant is a wild type plant. The control plant is typically of the same plant species, preferably having the same genetic background as the modified plant.

Genome Editing Constructs for Use with the Methods for Targeted Genome Modification Described Herein

By “crRNA” or CRISPR RNA is meant the sequence of RNA that contains the protospacer element and additional nucleotides that are complementary to the tracrRNA.

By “tracrRNA” (transactivating RNA) is meant the sequence of RNA that hybridises to the crRNA and binds a CRISPR enzyme, such as Cas9 thereby activating the nuclease complex to introduce double-stranded breaks at specific sites within the genomic sequence of at least one GSE5 or GSE5-Like nucleic acid or promoter sequence.

By “protospacer element” is meant the portion of crRNA (or sgRNA) that is complementary to the genomic DNA target sequence, usually around 20 nucleotides in length. This may also be known as a spacer or targeting sequence.

By “sgRNA” (single-guide RNA) is meant the combination of tracrRNA and crRNA in a single RNA molecule, preferably also including a linker loop (that links the tracrRNA and crRNA into a single molecule). “sgRNA” may also be referred to as “gRNA” and in the present context, the terms are interchangeable. The sgRNA or gRNA provide both targeting specificity and scaffolding/binding ability for a Cas nuclease. A gRNA may refer to a dual RNA molecule comprising a crRNA molecule and a tracrRNA molecule.

By “TAL effector” (transcription activator-like (TAL) effector) or TALE is meant a protein sequence that can bind the genomic DNA target sequence (a sequence within the GSE5 or GSE5-Like gene or promoter sequence) and that can be fused to the cleavage domain of an endonuclease such as Fokl to create TAL effector nucleases or TALENS or meganucleases to create megaTALs. A TALE protein is composed of a central domain that is responsible for DNA binding, a nuclear-localisation signal and a domain that activates target gene transcription. The DNA-binding domain consists of monomers and each monomer can bind one nucleotide in the target nucleotide sequence. Monomers are tandem repeats of 33-35 amino acids, of which the two amino acids located at positions 12 and 13 are highly variable (repeat variable diresidue, RVD). It is the RVDs that are responsible for the recognition of a single specific nucleotide. HD targets cytosine; NI targets adenine, NG targets thymine and NN targets guanine (although NN can also bind to adenine with lower specificity).

In another aspect of the invention there is provided a nucleic acid construct wherein the nucleic acid construct encodes at least one DNA-binding domain, wherein the DNA-binding domain can bind to a sequence in the GSE5 gene or GSE5-Like gene, wherein said sequence is selected from SEQ ID NOs: 15 to 20, 48, 51, 76, 79, 80, 81, 82, 83 and 84. In one embodiment, said construct further comprises a nucleic acid encoding a SSN, such as Fokl or a Cas protein.

In one embodiment, the nucleic acid construct encodes at least one protospacer element wherein the sequence of the protospacer element is selected from SEQ ID NOs: 21 to 26 or 52 or 77 or a variant thereof.

In a further embodiment, the nucleic acid construct comprises a crRNA-encoding sequence. As defined above, a crRNA sequence may comprise the protospacer elements as defined above and preferably additional nucleotides that are complementary to the tracrRNA. An appropriate sequence for the additional nucleotides will be known to the skilled person as these are defined by the choice of Cas protein.

In another embodiment, the nucleic acid construct further comprises a tracrRNA sequence. Again, an appropriate tracrRNA sequence would be known to the skilled person as this sequence is defined by the choice of Cas protein.

In a further embodiment, the nucleic acid construct comprises at least one nucleic acid sequence that encodes a sgRNA (or gRNA). Again, as already discussed, sgRNA typically comprises a crRNA sequence, a tracrRNA sequence and preferably a sequence for a linker loop. In a preferred embodiment, the nucleic acid construct comprises at least one nucleic acid sequence that encodes a sgRNA sequence as defined herein in SEQ ID NO: 78 or variant thereof.

In a further embodiment, the nucleic acid construct may further comprise at least one nucleic acid sequence encoding an endoribonuclease cleavage site. Preferably the endoribonuclease is Csy4 (also known as Cas6f). Where the nucleic acid construct comprises multiple sgRNA nucleic acid sequences the construct may comprise the same number of endoribonuclease cleavage sites. In another embodiment, the cleavage site is 5′ of the sgRNA nucleic acid sequence. Accordingly, each sgRNA nucleic acid sequence is flanked by a endoribonuclease cleavage site.

The term ‘variant’ refers to a nucleotide sequence where the nucleotides are substantially identical to one of the above sequences. The variant may be achieved by modifications such as an insertion, substitution or deletion of one or more nucleotides. In a preferred embodiment, the variant has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to any one of the above sequences. In one embodiment, sequence identity is at least 90%. In another embodiment, sequence identity is 100%. Sequence identity can be determined by any one known sequence alignment program in the art.

The invention also relates to a nucleic acid construct comprising a nucleic acid sequence operably linked to a suitable plant promoter. A suitable plant promoter may be a constitutive or strong promoter or may be a tissue-specific promoter. In one embodiment, suitable plant promoters are selected from, but not limited to U3 and U6.

The nucleic acid construct of the present invention may also further comprise a nucleic acid sequence that encodes a CRISPR enzyme. By “CRISPR enzyme” is meant an RNA-guided DNA endonuclease that can associate with the CRISPR system. Specifically, such an enzyme binds to the tracrRNA sequence. In one embodiment, the CRIPSR enzyme is a Cas protein (“CRISPR associated protein), preferably Cas 9 or Cpf1, more preferably Cas9. In a specific embodiment Cas9 is a codon-optimised Cas9 (specific for the plant in question). In one embodiment, Cas9 has the sequence described in SEQ ID NO: 33 or a functional variant or homolog thereof. In another embodiment, the CRISPR enzyme is a protein from the family of Class 2 candidate x proteins, such as C2c1, C2C2 and/or C2c3. In one embodiment, the Cas protein is from Streptococcus pyogenes. In an alternative embodiment, the Cas protein may be from any one of Staphylococcus aureus, Neisseria meningitides, Streptococcus thermophiles or Treponema denticola.

The term “functional variant” as used herein with reference to Cas9 refers to a variant Cas9 gene sequence or part of the gene sequence which retains the biological function of the full non-variant sequence, for example, acts as a DNA endonuclease, or recognition or/and binding to DNA. A functional variant also comprises a variant of the gene of interest which has sequence alterations that do not affect function, for example non-conserved residues. Also encompassed is a variant that is substantially identical, i.e. has only some sequence variations, for example in non-conserved residues, compared to the wild type sequences as shown herein and is biologically active. In one embodiment, a functional variant of SEQ ID NO: 33 has at least 90%, 91%, 92%, 93%, 94%, 95%, 96%, 97%, 98%, or 99% overall sequence identity to the amino acid represented by SEQ ID NO: 33. In a further embodiment, the Cas9 protein has been modified to improve activity.

Suitable homologs or orthologs can be identified by sequence comparisons and identifications of conserved domains. The function of the homolog or ortholog can be identified as described herein and a skilled person would thus be able to confirm the function when expressed in a plant.

In an alternative aspect of the invention, the nucleic acid construct comprises at least one nucleic acid sequence that encodes a TAL effector, wherein said effector targets a GSE5 sequence selected from SEQ ID NOs: 15 to 20 or 48 or 51 or a GSE5-Like sequence selected from SEQ ID NOs 76 and 79 to 84. Methods for designing a TAL effector would be well known to the skilled person, given the target sequence. Examples of suitable methods are given in Sanjana et al., and Cermak T et al, both incorporated herein by reference. Preferably, said nucleic acid construct comprises two nucleic acid sequences encoding a TAL effector, to produce a TALEN pair. In a further embodiment, the nucleic acid construct further comprises a sequence-specific nuclease (SSN). Preferably such SSN is a endonuclease such as Fokl. In a further embodiment, the TALENs are assembled by the Golden Gate cloning method in a single plasmid or nucleic acid construct.

In another aspect of the invention, there is provided a sgRNA molecule, wherein the sgRNA molecule comprises a crRNA sequence and a tracrRNA sequence and wherein the crRNA sequence can bind to at least one sequence selected from SEQ ID NOs: 15 to 20, 48, 51, 76 or 79 to 84 or a variant thereof.

A “variant” is as defined herein. In one embodiment, the sgRNA molecule may comprise at least one chemical modification, for example that enhances its stability and/or binding affinity to the target sequence or the crRNA sequence to the tracrRNA sequence. Such modifications would be well known to the skilled person, and include for example, but not limited to, the modifications described in Randar et al., 2015, incorporated herein by reference. In this example the crRNA may comprise a phosphorothioate backbone modification, such as 2′-fluoro (2′-F), 2′-O-methyl (2′-O-Me) and S-constrained ethyl (cET) substitutions.

In another aspect of the invention, there is provided an isolated nucleic acid sequence that encodes for a protospacer element (as defined in any of SEQ ID NOs: 21 to 26 or 52 or 77), or a sgRNA.

In another aspect of the invention, there is provided a plant or part thereof or at least one isolated plant cell transfected with at least one nucleic acid construct as described herein. Cas9 and sgRNA may be combined or in separate expression vectors (or nucleic acid constructs, such terms are used interchangeably). In other words, in one embodiment, an isolated plant cell is transfected with a single nucleic acid construct comprising both sgRNA and Cas9 as described in detail above. In an alternative embodiment, an isolated plant cell is transfected with two nucleic acid constructs, a first nucleic acid construct comprising at least one sgRNA as defined above and a second nucleic acid construct comprising Cas9 or a functional variant or homolog thereof. The second nucleic acid construct may be transfected below, after or concurrently with the first nucleic acid construct. The advantage of a separate, second construct comprising a cas protein is that the nucleic acid construct encoding at least one sgRNA can be paired with any type of cas protein, as described herein, and therefore is not limited to a single cas function (as would be the case when both cas and sgRNA are encoded on the same nucleic acid construct).

In one embodiment, the nucleic acid construct comprising a cas protein is transfected first and is stably incorporated into the genome, before the second transfection with a nucleic acid construct comprising at least one sgRNA nucleic acid. In an alternative embodiment, a plant or part thereof or at least one isolated plant cell is transfected with mRNA encoding a cas protein and co-transfected with at least one nucleic acid construct as defined herein.

Cas9 expression vectors for use in the present invention can be constructed as described in the art. In one example, the expression vector comprises a nucleic acid sequence as defined herein or a functional variant or homolog thereof, wherein said nucleic acid sequence is operably linked to a suitable promoter. Examples of suitable promoters include, but are not limited to Cas9, 35S and Actin.

In an alternative aspect of the present invention, there is provided an isolated plant cell transfected with at least one sgRNA molecule as described herein.

In a further aspect of the invention, there is provided a genetically modified or edited plant comprising the transfected cell described herein. In one embodiment, the nucleic acid construct or constructs may be integrated in a stable form. In an alternative embodiment, the nucleic acid construct or constructs are not integrated (i.e. are transiently expressed). Accordingly, in a preferred embodiment, the genetically modified plant is free of any sgRNA and/or Cas protein nucleic acid. In other words, the plant is transgene free.

The term “introduction”, “transfection” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The resulting transformed plant cell may then be used to regenerate a transformed plant in a manner known to persons skilled in the art. The transfer of foreign genes into the genome of a plant is called transformation.

Transformation of plants is now a routine technique in many species. Any of several transformation methods known to the skilled person may be used to introduce the nucleic acid construct or sgRNA molecule of interest into a suitable ancestor cell. The methods described for the transformation and regeneration of plants from plant tissues or plant cells may be utilized for transient or for stable transformation.

Transformation methods include the use of liposomes, electroporation, chemicals that increase free DNA uptake, injection of the DNA directly into the plant (microinjection), gene guns (or biolistic particle delivery systems (bioloistics)) as described in the examples, lipofection, transformation using viruses or pollen and microprojection. Methods may be selected from the calcium/polyethylene glycol method for protoplasts, ultrasound-mediated gene transfection, optical or laser transfection, transfection using silicon carbide fibers, electroporation of protoplasts, microinjection into plant material, DNA or RNA-coated particle bombardment, infection with (non-integrative) viruses and the like. Transgenic plants, can also be produced via Agrobacterium tumefaciens mediated transformation, including but not limited to using the floral dip/Agrobacterium vacuum infiltration method as described in Clough & Bent (1998) and incorporated herein by reference.

Accordingly, in one embodiment, at least one nucleic acid construct or sgRNA molecule as described herein can be introduced to at least one plant cell using any of the above described methods. In an alternative embodiment, any of the nucleic acid constructs described herein may be first transcribed to form a preassembled Cas9-sgRNA ribonucleoprotein and then delivered to at least one plant cell using any of the above described methods, such as lipofection, electroporation or microinjection.

Optionally, to select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility is growing the seeds, if appropriate after sterilization, on agar plates using a suitable selection agent so that only the transformed seeds can grow into plants. As described in the examples, a suitable marker can be bar-phosphinothricin or PPT. Alternatively, the transformed plants are screened for the presence of a selectable marker, such as, but not limited to, GFP, GUS (β-glucuronidase). Other examples would be readily known to the skilled person. Alternatively, no selection is performed, and the seeds obtained in the above-described manner are planted and grown and GSE5 expression or protein levels measured at an appropriate time using standard techniques in the art. This alternative, which avoids the introduction of transgenes, is preferable to produce transgene-free plants.

Following DNA transfer and regeneration, putatively transformed plants may also be evaluated, for instance using PCR to detect the presence of the gene of interest, copy number and/or genomic organisation. Alternatively or additionally, integration and expression levels of the newly introduced DNA may be monitored using Southern, Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques.

In a further related aspect of the invention, there is also provided, a method of obtaining a genetically modified plant as described herein, the method comprising
a. selecting a part of the plant;
b. transfecting at least one cell of the part of the plant of paragraph (a) with at least one nucleic acid construct as described herein or at least one sgRNA molecule as described herein, using the transfection or transformation techniques described above;
c. regenerating at least one plant derived from the transfected cell or cells;
d. selecting one or more plants obtained according to paragraph (c) that show silencing or reduced expression of GSE5 or GSE5-Like.

In a further embodiment, the method also comprises the step of screening the genetically modified plant for SSN (preferably CRISPR)-induced mutations in the GSE5 gene or promoter sequence. In one embodiment, the method comprises obtaining a DNA sample from a transformed plant and carrying out DNA amplification to detect a mutation in at least one GSE5 or GSE5-Like gene or promoter sequence.

In a further embodiment, the methods comprise generating stable T2 plants preferably homozygous for the mutation (that is a mutation in at least one GSE5 or GSE5-Like gene or promoter sequence).

Plants that have a mutation in at least one GSE5 or GSE5-Like gene and/or promoter sequence can also be crossed with another plant also containing at least one mutation in at least one GSE5 or GSE5-Like gene and/or promoter sequence to obtain plants with additional mutations in the GSE5 gene or GSE5-Like or promoter sequence. The combinations will be apparent to the skilled person. Accordingly, this method can be used to generate a T2 plants with mutations on all or an increased number of homoeologs, when compared to the number of homoeolog mutations in a single T1 plant transformed as described above.

A plant obtained or obtainable by the methods described above is also within the scope of the invention.

A genetically altered plant of the present invention may also be obtained by transference of any of the sequences of the invention by crossing, e.g., using pollen of the genetically altered plant described herein to pollinate a wild-type or control plant, or pollinating the gynoecia of plants described herein with other pollen that does not contain a mutation in at least one of the GSE5 or GSE5-Like gene or promoter sequence. The methods for obtaining the plant of the invention are not exclusively limited to those described in this paragraph; for example, genetic transformation of germ cells from the ear of wheat could be carried out as mentioned, but without having to regenerate a plant afterward.

### Method of Screening Plants for Naturally Occurring Low Levels of GSE5 Expression

In a further aspect of the invention, there is provided a method for screening a population of plants and identifying and/or selecting a plant that will have reduced GSE5 or GSE5-Like expression and/or an increased seed yield phenotype, preferably an increased seed width, weight or TKW, the method comprising detecting in the plant or plant germplasm at least one polymorphism (preferably a low GSE5 or GSE5-Like expresser polymorphism) in the promoter of the GSE5 or GSE5-Like gene. Preferably, said screening comprises determining the presence of at least one polymorphism, wherein said polymorphism is at least one insertion and/or at least one deletion.

In one embodiment, a plant expressing a deletion of a nucleic acid sequence comprising SEQ ID NO: 30 will express ˜0.6 fold lower level of GSE5 expression compared to a plant wherein the promoter without this polymorphism. In one embodiment, the plant is rice, preferably the japonica variety. Such plants are referred to herein as GSE5^{DEL2}.

In another embodiment, a plant expressing a deletion of a nucleic acid sequence comprising SEQ ID NO: 29 and/or the insertion of a nucleic acid sequence comprising SEQ ID NO: 31 will express ˜0.65 fold lower level of GSE5 expression compared to a plant wherein the promoter without this polymorphism. In one embodiment, the plant is rice, preferably the indica variety. Such plants are referred to herein as GSE5^{DEL1+IN1}.

As a result, the above-described plants will display an increased seed yield as described above.

Suitable tests for assessing the presence of a polymorphism would be well known to the skilled person, and include but are not limited to, Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs-which are also referred to as Microsatellites), and Single Nucleotide Polymorphisms (SNPs). In one embodiment, Kompetitive Allele Specific PCR (KASP) genotyping is used.

In one embodiment, the method comprises

a) obtaining a nucleic acid sample from a plant and
b) carrying out nucleic acid amplification of one or more GSE5 promoter alleles using one or more primer pairs.

In a further embodiment, the method may further comprise introgressing the chromosomal region comprising at least one of said low-GSE5-expressing polymorphisms or the chromosomal region containing the repeat sequence deletion as described above into a second plant or plant germplasm to produce an introgressed plant or plant germplasm. Preferably the expression of GSE5 or GSE5-Like in said second plant will be reduced or abolished, and more preferably said second plant will display an increase in seed size, and increase in total protein and/or lipid content and/or a reduction in glucosinolate levels.

In one embodiment, plants of the GSE5^{DEL2} and GSE5^{DEL1+IN1} haplotypes may be selected and the levels of GSE5 nucleic acid and/or activity of the GSE5 protein reduced or further reduced by any method described herein.

Accordingly, in a further aspect of the invention there is provided a method for increasing yield, preferably seed or grain yield in a plant, the method comprising
a. screening a population of plants for at least one plant with the GSE5^{DEL2} and GSE5^{DEL1+IN1} haplotype; and
b. further reducing or abolishing the expression of at least one GSE5 nucleic acid and/or reducing the activity of a GSE5 polypeptide in said plant by introducing at least one mutation into the nucleic acid sequence encoding GSE5 or at least one mutation into the promoter of GSE5 as described herein or using RNA interference as described herein.

By “further reducing” is meant reducing the level of GSE5 expression to a level lower than that in the plant with the GSE5^{DEL2} and GSE5^{DEL1+IN1} haplotype in step a. The terms “reducing” means a decrease in the levels of GSE5 expression and/or activity by up to 10%, 20%, 30%, 40%, 50%, 60%, 70%, 80% or 90% when compared to the level in a GSE5^{DEL2} and GSE5^{DEL1+IN1} control plant.

### Methods of Increasing Grain Length

The inventors have also surprisingly identified that increasing the expression of GSE5 or GSE5-Like results in slender grains—i.e. an increase in grain length.

The terms “increase”, “improve” or “enhance” as used herein are interchangeable. In one embodiment, grain length is increased by at least 2%, 3%, 4%, 5%, 6%, 7%, 8%, 9%, 10% 11%, 12%, 13%, 14%, 15%, 16%, 17%, 18%, 19%, 20%, 30%, 40%, 50%, 55%, 60%, 65%, 70%, 75%, 80%, 85%, 90%, 95%, 100%, 105%, 110%, 120% or more in comparison to a control plant. Preferably, the increase is at least 2-10%, more preferably 3-8%.

Accordingly, in another aspect of the invention there is provided a nucleic acid construct comprising a nucleic acid sequence encoding a polypeptide as defined in SEQ ID NO: 1 or 57 or a functional variant or homolog thereof, wherein said sequence is operably linked to a regulatory sequence, wherein preferably said regulatory sequence is a tissue-specific promoter or a constitutive promoter. In a further embodiment, the nucleic acid construct comprises a nucleic acid sequence as defined in SEQ ID NO: 2 or 56 (cDNA) or 32 or 55 (genomic) or a functional variant or homolog thereof. A functional variant or homolog is as defined above.

The term “operably linked” as used herein refers to a functional linkage between the promoter sequence and the gene of interest, such that the promoter sequence is able to initiate transcription of the gene of interest.

A “plant promoter” comprises regulatory elements, which mediate the expression of a coding sequence segment in plant cells. Accordingly, a plant promoter need not be of plant origin, but may originate from viruses or micro-organisms, for example from viruses which attack plant cells. The “plant promoter” can also originate from a plant cell, e.g. from the plant which is transformed with the nucleic acid sequence to be expressed in the inventive process and described herein. This also applies to other “plant” regulatory signals, such as “plant” terminators. The promoters upstream of the nucleotide sequences useful in the methods of the present invention can be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoters is increased by modification of their sequence, or that they are replaced completely by more active promoters, even promoters from heterologous organisms. For expression in plants, the nucleic acid molecule must, as described above, be linked operably to or comprise a suitable promoter which expresses the gene at the right point in time and with the required spatial expression pattern. The term “operably linked” as used herein refers to a functional linkage between the promoter sequence and the gene of interest, such that the promoter sequence is able to initiate transcription of the gene of interest.

In one embodiment, the promoter is a constitutive promoter. A “constitutive promoter” refers to a promoter that is transcriptionally active during most, but not necessarily all, phases of growth and development and under most environmental conditions, in at least one cell, tissue or organ. Examples of constitutive promoters include but are not limited to actin, HMGP, CaMV19S, GOS2, rice cyclophilin, maize H3 histone, alfalfa H3 histone, 34S FMV, rubisco small subunit, OCS, SAD1, SAD2, nos, V-ATPase, super promoter, G-box proteins and synthetic promoters.

In another aspect of the invention there is provided a vector comprising the nucleic acid sequence described above.

In a further aspect of the invention, there is provided a host cell comprising the nucleic acid construct. The host cell may be a bacterial cell, such as Agrobacterium tumefaciens, or an isolated plant cell. The invention also relates to a culture medium or kit comprising a culture medium and an isolated host cell as described below.

In another embodiment, there is provided a transgenic plant expressing the nucleic acid construct as described above. In one embodiment, said nucleic acid construct is stably incorporated into the plant genome.

The nucleic acid sequence is introduced into said plant through a process called transformation as described above.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

A suitable plant is defined above.

In another aspect, the invention relates to the use of a nucleic acid construct as described herein to increase grain length as defined above.

In a further aspect of the invention there is provided a method of increasing grain length, the method comprising introducing and expressing in said plant the nucleic acid construct described herein.

In another aspect of the invention there is provided a method of producing a plant with an increased grain length the method comprising introducing and expressing in said plant the nucleic acid construct described herein.

Said increase is relative to a control or wild-type plant.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

The foregoing application, and all documents and sequence accession numbers cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

The invention is now described in the following non-limiting example.

### EXAMPLE

The utilization of natural genetic variation greatly contributes to improvement of important agronomic traits in crops. Understanding the genetic basis for natural variation of grain size can help breeders develop high-yield rice varieties. Here we identify a novel quantitative trait locus for grain size (GSE5) using a genome-wide association study (GWAS) with functional testing. GSE5 encodes a plasma membrane-associated protein with IQ domains (IQD), which associates with calmodulin (OsCaM1-1). GSE5 regulates grain size by influencing cell proliferation. We identify three major haplotypes (GSE5, GSE5^{DEL1+IN1} and GSE5^{DEL2}) in cultivated rice according to the deletion/insertion type in the promoter of GSE5. We demonstrate that the deletion 1 (DEL1) in indica varieties carrying the GSE5^{DEL1+IN1} haplotype and the deletion 2 (DEL2) in japonica varieties carrying the GSE5^{DEL2} haplotype cause the decreased expression of GSE5, resulting in wide grains. We generate loss-of-function mutant of GSE5 that increases grain width and weight, while overexpression of GSE5 results in slender grains. Further analyses indicate that wild rice accessions contain GSE5, GSE5^{DEL1+IN1} and GSE5^{DEL2} haplotypes, suggesting that these three major haplotypes in cultivated rice are likely to have originated from different wild rice accessions during rice domestication. Thus, these findings identify a novel QTL gene for grain size (GSE5) that is widely utilized by rice breeders and reveal that natural variation in the promoter of GSE5 contributes to grain size diversity in rice.

### Results and Discussion

### Identification of the GSE5 Locus by the GWAS Analysis

To identify natural variation in genes involved in grain size control, we performed the genome-wide association study (GWAS) with functional analysis. We used 102 indica varieties, which showed large variation in grain size (FIG. 7). To detect nucleotide polymorphisms, we conducted whole-genome sequencing of these 102 indica varieties and got a total of 677.3 Gb of genomic sequence. The average sequencing depth is 15.4×, and 96.4% of the reference genome sequence is covered (International Rice Genome Sequencing, 2005). A total of 831,050 single nucleotide polymorphisms (SNPs) were detected among 102 indica varieties. Based on these nucleotide polymorphisms, we conducted principal component analysis (PCA) to characterize the population structure of these 102 indica varieties. These 102 indica varieties did not show a highly structured population. We then analyzed LD for these 102 indica varieties using these SNPs. The average decay of LD was about 220 kb in this population (r²=0.2) (which is similar to that of a previous study in rice (Huang et al., 2010).

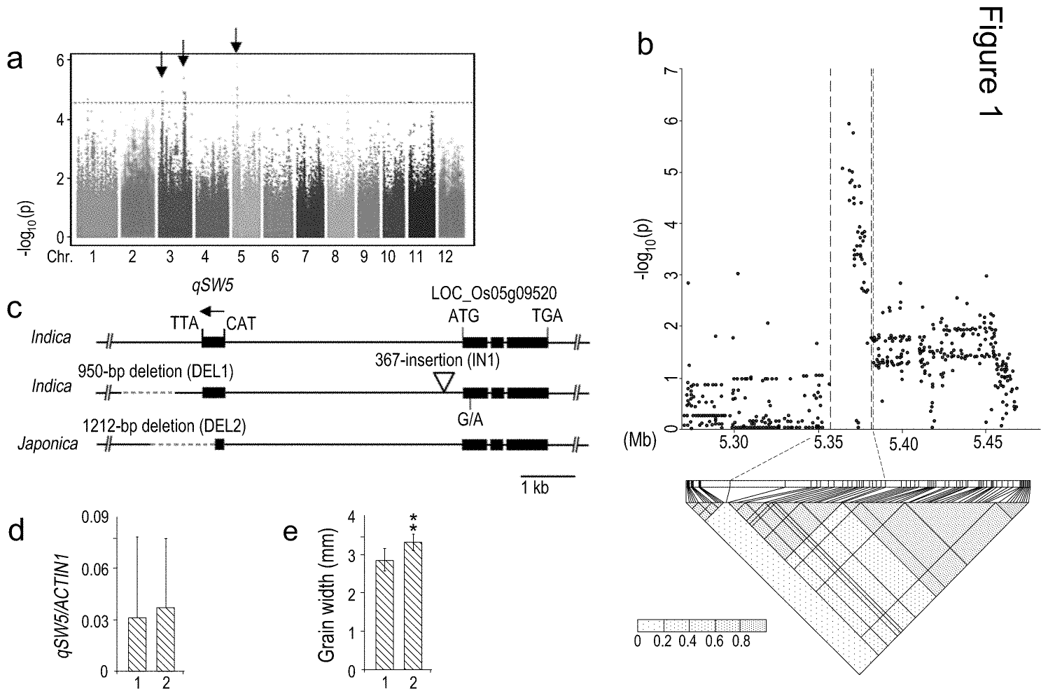

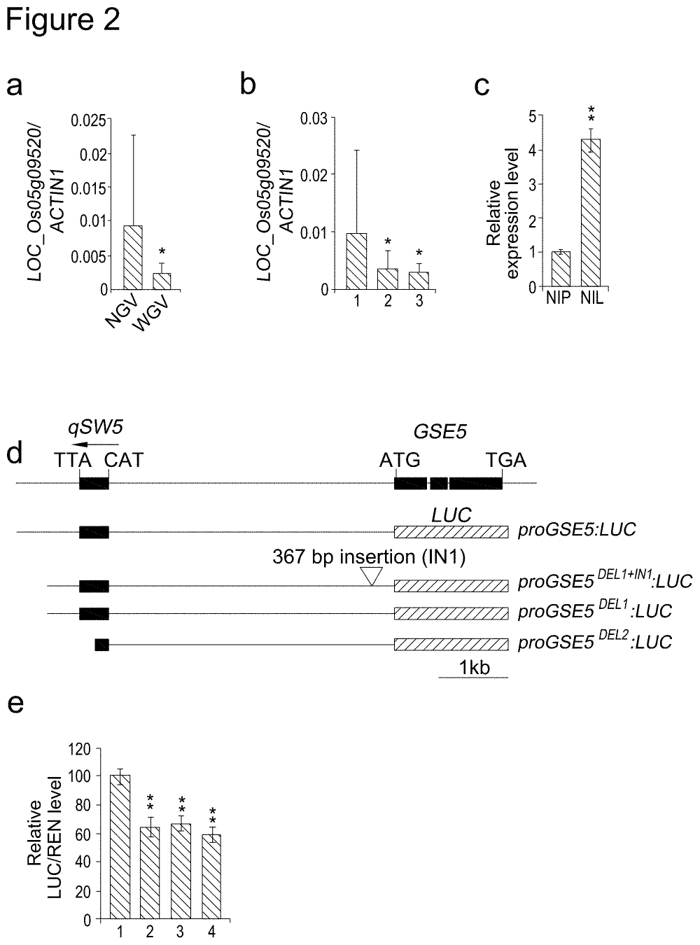

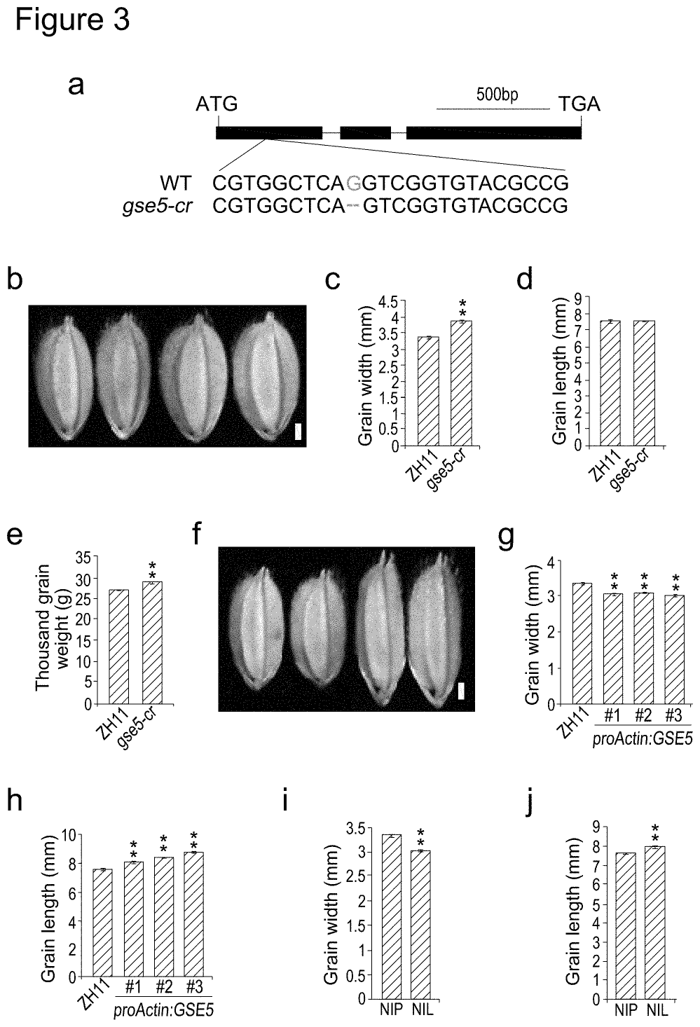

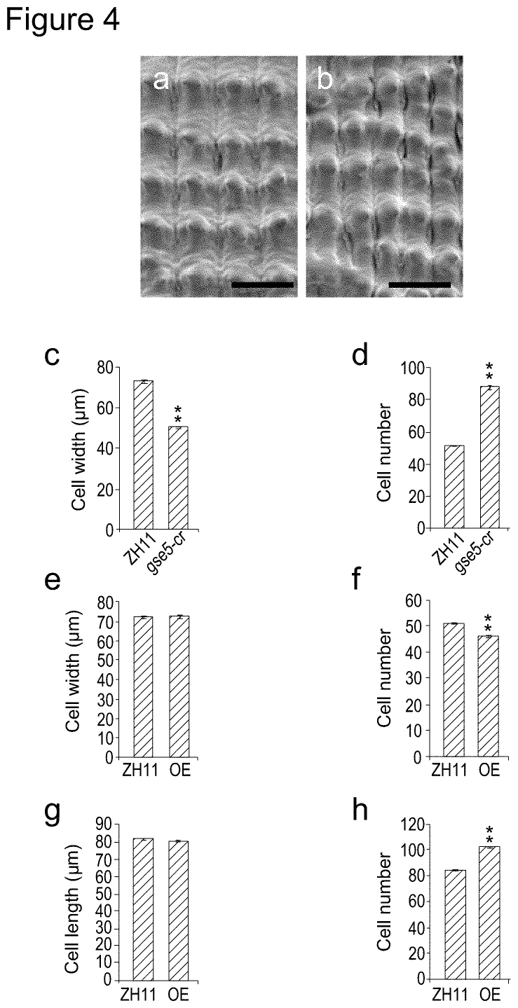

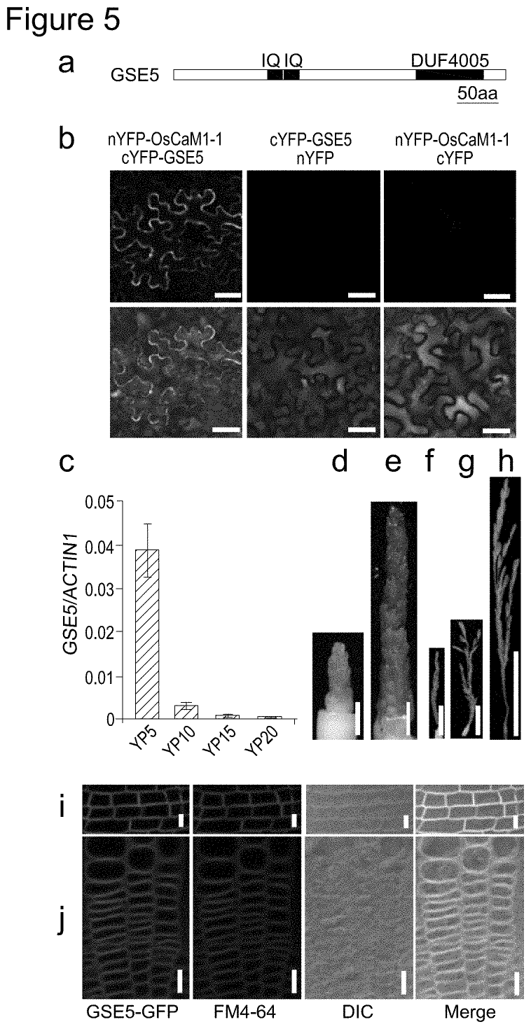

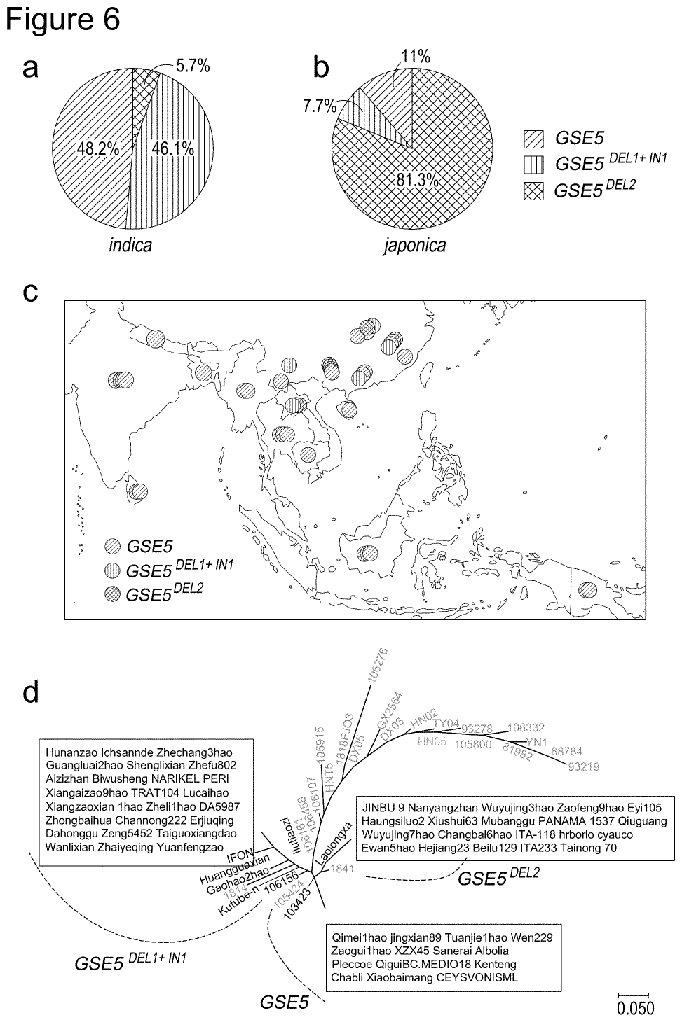

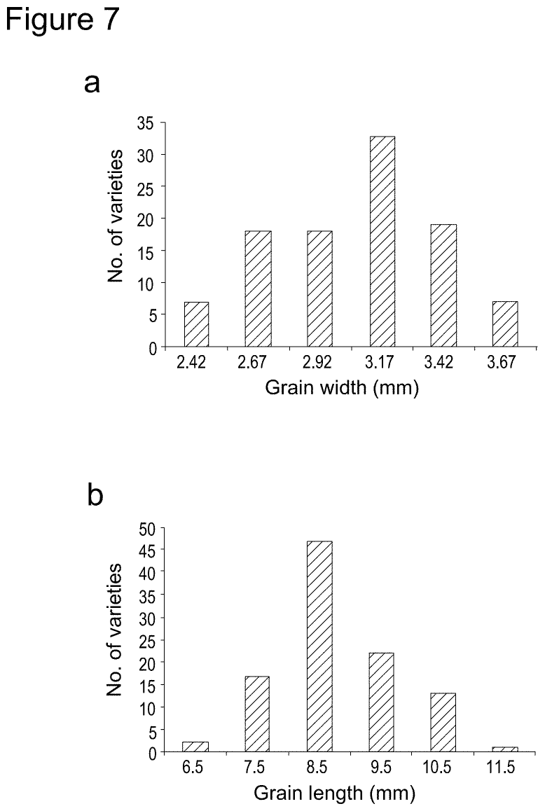

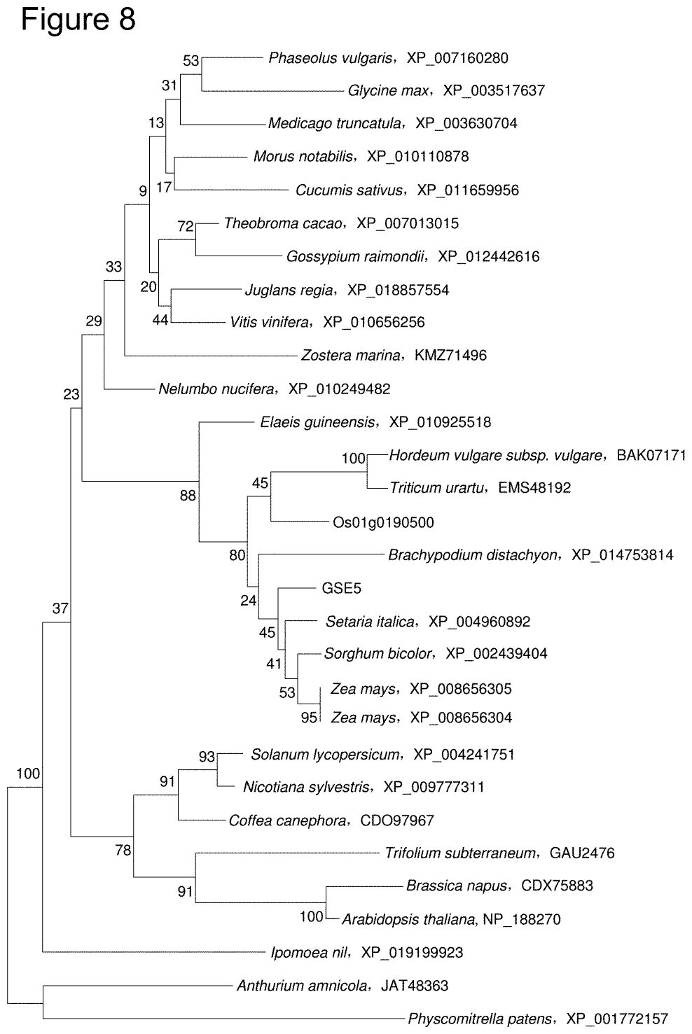

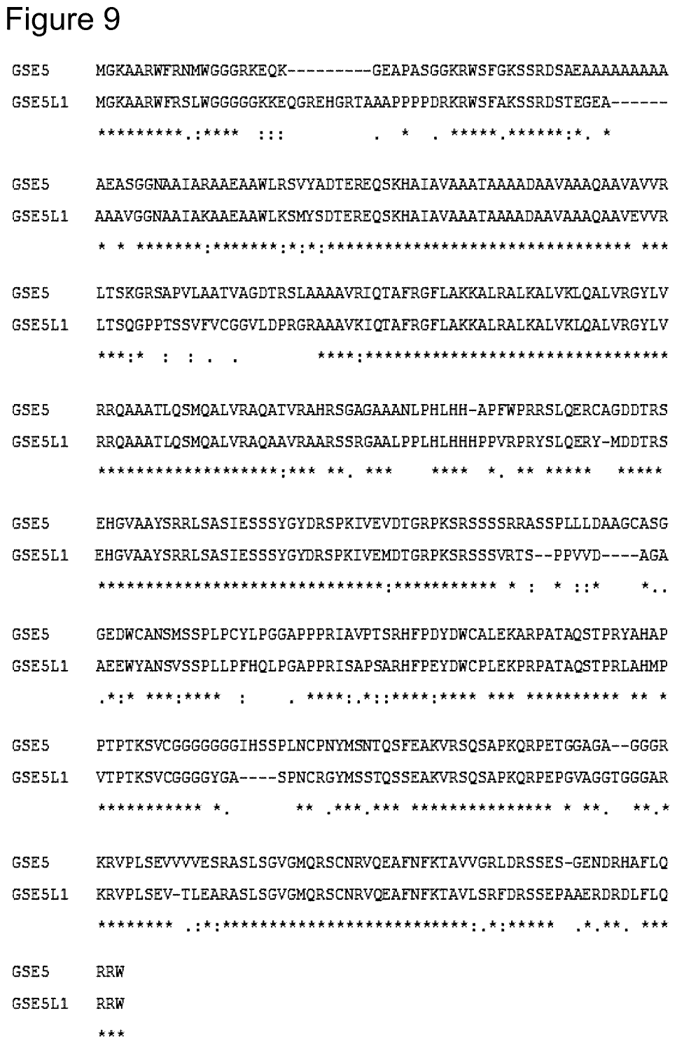

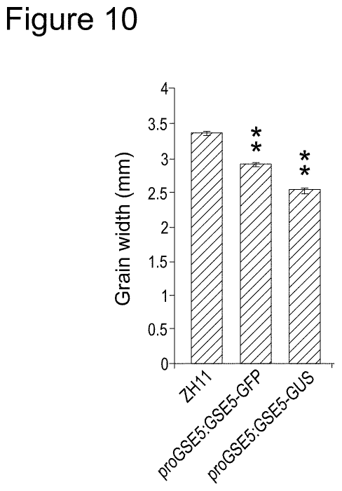

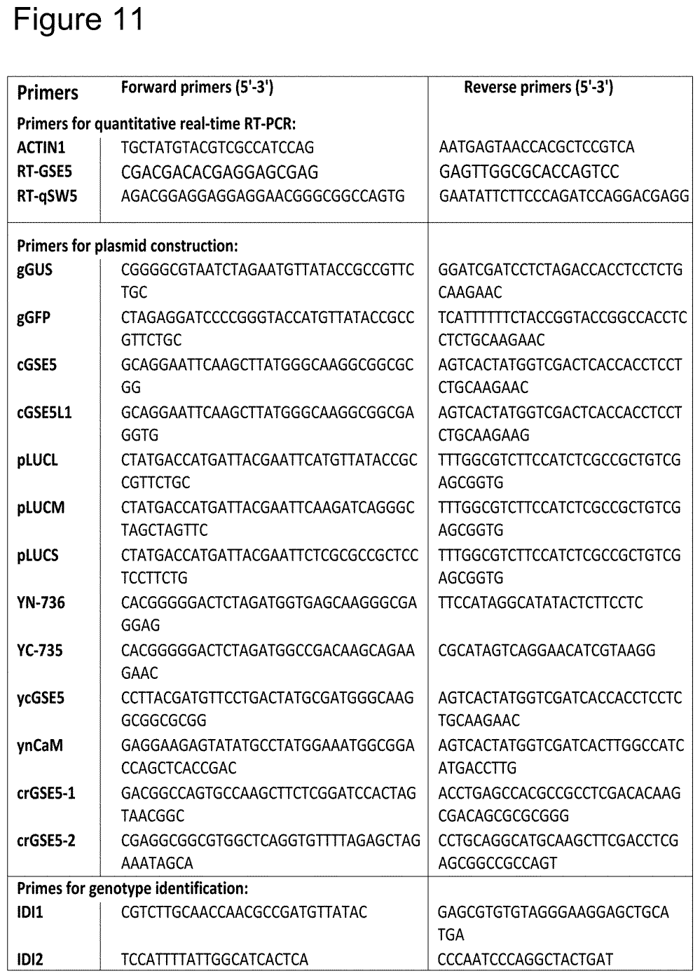

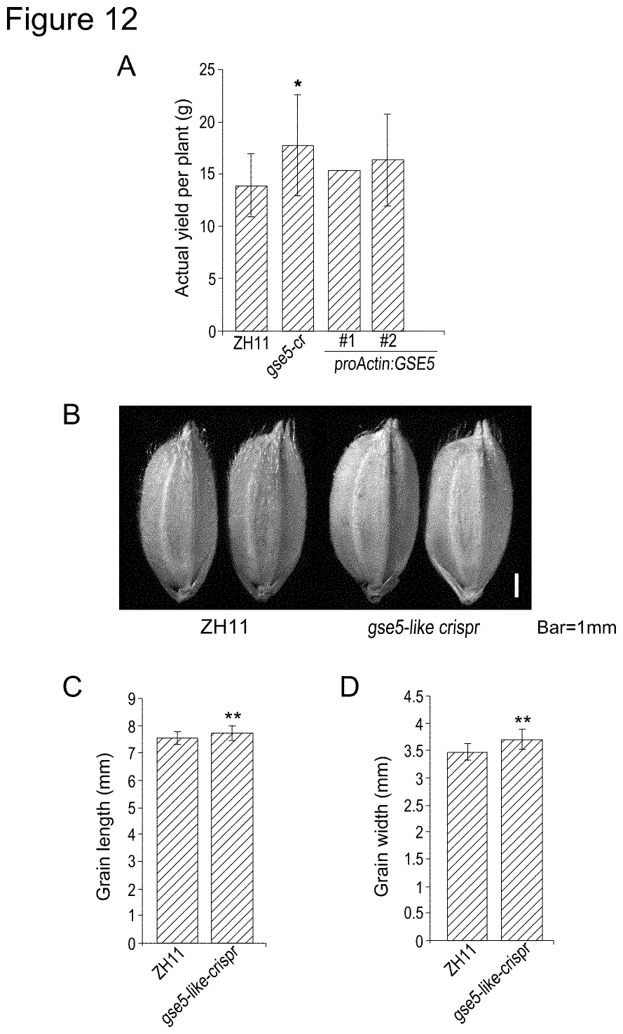

We performed GWAS for grain width in this indica population using a mixed linear model with correction of kinship, which is a widely used method for GWAS analysis (Huang et al., 2010; Yano et al., 2016). As shown in FIG. 1a, three loci were significantly associated with grain width. Surprisingly, one locus for grain width was located in the region of qSW5/GW5 on Chromosome 5, which has been known to determine grain-width differences between indica varieties and japonica varieties (Shomura et al., 2008; Weng et al., 2008). This suggests that qSW5 might not be responsible for grain width variation among these indica varieties. Most japonica varieties had a 1212-bp deletion (DEL2) in the qSW5 gene (FIG. 1c), resulting in wide grains (Shomura et al., 2008; Weng et al., 2008). Some indica varieties had no deletion in qSW5, while some indica varieties contained a 950-bp deletion (DEL1) in the 3′ flanking region of qSW5 (Shomura et al., 2008; Weng et al., 2008) (FIG. 1c). If this DEL1 affects the function of qSW5 in indica varieties, we presumed that it might decrease expression of qSW5. However, DEL1 was not associated with expression levels of qSW5 (FIG. 1d) in indica varieties, suggesting that DEL1 might not affect the function of qSW5. Thus, it is unlikely that qSW5 could be responsible for grain width differences among these indica varieties. Considering that DEL1 was strongly associated with grain width in indica varieties (FIG. 1e), the other gene in this locus could be responsible for grain width variation in indica varieties. We therefore designated this gene as GRAIN SIZE ON CHROMOSOME 5 (GSE5).

Expression Level of LOC_Os05g09520 is Associated with Grain Width

To identify the GSE5 gene, we used pairwise LD correlations (r²>0.6) (Yano et al., 2016) to estimate a candidate region from 5.357 Mb to 5.379 Mb (22.42 kb) (FIG. 1b). There are two genes within this 22.42-kb interval, including qSW5 and LOC_Os05g09520 (FIGS. 1b and 1c). This result suggests that LOC_Os05g09520 is a candidate gene for GSE5. We therefore sequenced the LOC_Os05g09520 gene in wide grain and narrow grain indica varieties, respectively. Although we found one SNP (G/A) in its coding region in wide grain varieties, it does not cause amino acid change (FIG. 1c). We then selected twenty narrow grain and wide grain indica varieties and examined expression levels of LOC_Os05g09520. As shown in FIG. 2a, expression levels of LOC_Os05g09520 were significantly associated with grain width. The LOC_Os05g09520 gene showed lower expression in wide grain indica varieties than that in narrow grain indica varieties, suggesting that the reduced expression of LOC_Os05g09520 might cause wide grains.

### DEL1 in Indica Varieties and DEL2 in Japonica Varieties Result in the Reduced Expression of LOC_Os05g09520, Respectively

To understand why expression of LOC_Os05g09520 is decreased in wide grain varieties, we examined the 5′-flanking sequences of LOC_Os05g09520 in indica varieties and found that most wide grain indica varieties contain a 950-bp deletion (DEL1) as well as a 367-bp insertion (IN1) (FIG. 1c). Thus, it is possible that DEL1 and IN1 might cause the decreased expression of LOC_Os05g09520 in wide grain indica varieties. As expected, DEL1 and IN1 negatively correlated with expression levels of LOC_Os05g09520 in indica varieties (FIG. 2b).

As the japonica varieties had the 1212-bp deletion (DEL2) that partially overlaps with DEL1 (FIG. 1c), we asked whether DEL2 could also associate with expression levels of LOC_Os05g09520 in rice. As shown in FIG. 2b, DEL2 was significantly associated with lower expression levels of LOC_Os05g09520. To further confirm that DEL2 is associated with the decreased expression of LOC_Os05g09520 in japonica varieties, we obtained a near isogenic line (NIL), which contains the LOC_Os05g09520 allele from the narrow grain indica variety 93-11 in the japonica variety Nipponbare background. As shown in FIG. 2c, expression of LOC_Os05g09520 in Nipponbare with the deletion DEL2 was significantly decreased compared with that in NIL, suggesting that DEL2 in japonica varieties might cause lower expression of LOC_Os05g09520.

To determine whether DEL1 and IN1 in indica varieties and DEL2 in japonica varieties could decrease expression of LOC_Os05g09520, we investigated the activity of the promoter (proGSE5) without or with DEL1 and IN1 (proGSE5^{DEL1+IN1}), the only DEL1 (proGSE5^{DEL1}) and the DEL2 (proGSE5^{DEL2}), respectively (FIG. 2d). As shown in FIG. 2e, the proGSE5 promoter had stronger activity than proGSE5^{DEL1+IN1} and proGSE5^{DEL2}, showing that DEL1+IN1 and DEL2 decrease the promoter activity of LOC_Os05g09520. The proGSE5^{DEL1+IN1} activity was similar to that of proGSE5^{DEL1}, indicating that DEL1 decreases the promoter activity and IN1 might not influence the promoter activity. Thus, these results show that DEL1 in indica varieties and DEL2 in japonica varieties contribute to the decreased expression of LOC_Os05g09520, respectively.

### The Identity of the GSE5 Gene

To confirm that LOC_Os05g09520 is the GSE5 gene, we generated the loss-of-function mutant for LOC_Os05g09520 and performed a genetic complementation test.

The japonica variety Zhonghua 11 (ZH11) with the deletion DEL2 in the promoter of LOC_Os05g09520 had wide grains. Although the ZH11 promoter (proGSE5^{DEL2}) had reduced activity, it still possessed partial activity (FIG. 2e). We therefore presumed that the further disruption of the LOC_Os05g09520 gene using CRISPR/Cas9 could increase the width of ZH11 grains. The mutant for LOC_Os05g09520 generated by CRISPR/Cas9 (GSE5-cr) had a 1-bp deletion in the first exon, resulting in a reading frame shift (FIG. 3a). As expected, GSE5-cr mutant produced wider grains than ZH11 (FIG. 3b, 3c). The length of GSE5-cr grains was similar to that of ZH11 grains (FIG. 3d). The 1000-grain weight of GSE5-cr was significantly increased compared with that of ZH11 (FIG. 3e). We then expressed the LOC_Os05g09520 gene under an Actin promoter (proActin:GSE5) in ZH11 background. Transgenic plants produced narrower grains than ZH11 (FIG. 3f-3h), indicating that the LOC_Os05g09520 gene complemented the wide grain phenotype of ZH11. We observed that transgenic plants had long grains compared with ZH11. We further examined the grain size of a near isogenic line (NIL), which contains the GSE5 locus from the narrow grain indica variety 93-11 in the japonica variety Nipponbare background. NIL also showed narrower and longer grains than Nipponbare (FIGS. 3i and 3j), like those observed in proActin:GSE5 transgenic lines. It is possible that there is a balance mechanism between grain width and grain length (Wang et al., 2015b). Taken together, these results reveal that GSE5 is the LOC_Os05g09520 gene.

### GSE5 Regulates Gran Size by Influencing Cell Proliferation

The spikelet hull restricts the growth of a grain, which has been proposed to influence grain size in rice (Li and Li, 2016). Cell proliferation and cell expansion coordinately determine the growth of spikelet hulls. We therefore measured cell number and cell size in ZH11 and GSE5-cr spikelet hulls. The GSE5-cr spikelet hulls contained more epidermal cells than ZH11 spikelet hulls in the grain-width direction (FIG. 4a, 4b, 4d), indicating that GSE5 controls grain width by limiting cell proliferation. By contrast, epidermal cells in GSE5-cr spikelet hulls were narrower than those in ZH11 spikelet hulls (FIG. 4c), suggesting a possible compensation mechanism between cell proliferation and cell expansion. This compensation phenomenon was also found in several Arabidopsis seed size mutants (Xia et al., 2013).

We then investigated cell number and cell size in ZH11 and proActin:GSE5 spikelet hulls. As shown in FIG. 4e-4h, the proActin:GSE5 spikelet hulls had fewer cells in the grain-width direction and more cells in the grain-length direction than ZH11 spikelet hulls, while epidermal cell length and width in proActin:GSE5 spikelet hulls were similar to those in ZH11, consistent with the narrow and long grain phenotypes of GSE5-OE. Thus, these results indicate that GSE5 controls grain size predominantly by influencing cell proliferation.

GSE5 Encodes a Plasma Membrane-Associated Protein with IQ Domains (IQD)

Grain size and weight are important agronomic traits in crops. We identify a novel grain size gene (GSE5) that encodes a plasma membrane-associated protein with IQ domains (IQD), which interacts with calmodulin (OsCaM1-1). In rice, loss of GSE5 function causes wide and heavy grains, while overexpression of GSE5 results in narrow and long grains. By performing a BAST search in the databases, we found that GSE5 shares significantly similarity with its homologs in other crops, such as maize, wheat, sorghum and brachypodium. Our current knowledge of GSE5 functions suggest that GSE5 and its homologs in other crops or plant species could be used to engineer large and heavy seeds in these key crops. We could use CRISPR/Cas9 technology to knock-out GSE5 or its homologs in other crops to increase seed size and weight in these crops. We also could use RNAi technology to knock-down the expression of GSE5 or its homologs in crops to increase seed size and weight in these crops.

GSE5 encodes a predicted protein with IQ domains (IQD) (FIG. 5a). IQD proteins are an ancient family of calmodulin-binding proteins and regulate plant stress responses and plant development (Abel et al., 2005; Xiao et al., 2008). We therefore asked whether GSE5 could interact with rice calmodulin. As shown in FIG. 5b, GSE5 physically associated with rice calmodulin (OsCaM1-1) in vivo. It is possible that GSE5 might be involved in calcium signalling to regulate grain size in rice. In plants, how calcium signalling is involved in seed size control is totally unknown. This result provides a good starting point for future studies on the role of calcium signalling in seed size control. Proteins that share significant homology with GSE5 are found in plant species such as rice, wheat, maize, soybean and sorghum, but not animals (FIG. 8), suggesting the GSE5 homologues might control seed size in plants.

GSE5 transcripts were detected in developing panicles using quantitative real-time RT-PCR analysis (FIG. 5c). We generated the GSE5 promoter:GSE5-GUS fusion (proGSE5:GSE5-GUS) transgenic rice plants and examined its tissue-specific expression patterns. The proGSE5:GSE5-GUS transgenic plants showed narrow grains (FIG. 10), indicating that the GSE5-GUS fusion protein is a functional protein. GUS activity was detected in at the early stages of developing panicles and grains, while GUS activity was disappeared at the late stages of panicle and grain development (FIG. 5d-5h). The expression patterns of GSE5 are consistent with its role in cell proliferation.

To determine the subcellular localization of GSE5, we expressed a GSE5-GFP fusion protein under its own promoter (proGSE5:GSE5-GFP) in the japonica variety ZH11. The proGSE5:GSE5-GFP transgenic plants produced narrow grains compared with ZH11 (FIG. 10), showing that the GSE5-GFP is a functional fusion protein. GFP fluorescence in proGSE5:GSE5-GFP transgenic plants was detected in the cell periphery (FIG. 5i). Plasmolysis induced with a high sucrose level was used to determine whether GSE5-GFP is associated with the plasma membrane or cell walls. GSE5-GFP was detected in the shrunken plasma membrane (FIG. 5j). Considering that GSE5 has no the predicted transmembrane domain, GSE5 may be a plasma membrane-associated protein.

### Evolutionary Aspects of the GSE5 Locus

Based on the deletion/insertion type in the promoter of GSE5, we identified three major haplotypes (GSE5, GSE5^{DEL1+IN1} and GSE5^{DEL2}) in cultivated rice (FIG. 1c). As DEL1 in indica varieties carrying the GSE5^{DEL1+IN1} haplotype and DEL2 in japonica varieties carrying the GSE5^{DEL2} haplotype contribute to wide grains, we genotyped cultivated rice including 141 indica rice and 91 japonica rice. Among 141 indica varieties, 48.2%, 46.1% and 5.7% of them were GSE5, GSE5^{DEL1+IN1} and GSE5^{DEL2} haplotypes, respectively (FIG. 6a). By contrast, among 91 japonica varieties, 11%, 7.7% and 81.3% of them contained GSE5, GSE5^{DEL1+IN1} and GSE5^{DEL2} haplotypes, respectively (FIG. 6b). These results indicate that DEL1 in indica varieties and DEL2 in japonica varieties were widely utilized by rice breeders, respectively.

Cultivated rice has been proposed to be domesticated from wild rice (Oryza rufipogon). We therefore asked whether wild rice accessions could contain these two deletions (DEL1 and DEL2) in the promoter region of GSE5. We genotyped 41 wild rice accessions (O. rufipogon) and observed that most accessions had the GSE5 haplotype, five accessions contained the GSE5^{DEL1+IN1} haplotype, and only one wild rice accession that came from Hunan province in the south region of China had the GSE5^{DEL2} haplotype (FIG. 6c). This result shows that these two major deletions (DEL1 and DEL2) might have occurred before the domestication of cultivated rice. Phylogenetic analyses of the GSE5 locus of 63 cultivated rice and 26 O. rufipogon accessions showed that several wild rice accessions were clustered together with cultivated rice varieties carrying GSE5, GSE5^{DEL1+IN1} or GSE5^{DEL2} haplotypes, respectively (FIG. 6d). These results suggest that the GSE5, GSE5^{DEL1+IN1} and GSE5^{DEL2} haplotypes in cultivated rice are likely to have originated from different O. rufipogon accessions during rice domestication.

In summary, our findings identify a novel quantitative trait gene for grain size (GSE5) using a genome-wide association study with functional testing, which is widely utilized by rice breeders. We demonstrate that natural variation in the promoter of GSE5 contributes to grain size diversity in cultivated rice. Our findings provide insight into the genetic basis for natural variation in rice grain size control.

### Methods

### Plant Materials and Growth Conditions

The cultivated rice varieties were obtained from a collection of cultivated rice preserved at the China National Rice Research Institute. The common wild rice varieties (Oryza rufipogon) were obtained from the Institute of Botany, Chinese Academy of Sciences (Zheng and Ge, 2010; Zhu et al., 2007). The indica and japonica varieties used in this study were cultivated in the paddy fields at Hangzhou (China) and Hainan (China).

### Morphological and Cellular Analyses

Grain size of the 102 indica varieties was measured using the SC Detection and Analysis System of Rice Seeds (Hangzhou WSeen Detection Technology). Dry grains of Zhonghua 11 (ZH11) and GSE5-cr were weighted using electronic analytical balance (METTLER MOLEDO AL104 CHINA).

To observe cell size and cell number, grain hulls of Zhonghua 11 (ZH11), GSE5-cr and proActin:GSE5 transgenic plants were sputter-coated with platinum and observed using a scanning electron microscope (SEM) (HITACHI S-3000N). Image J software was used to measure epidermal cell size.

### DNA Isolation, Genome Sequencing and Sequence Analysis

NuClean PlantGen DNA kits (CWBIO, China) were used for the genomic DNA extraction. For each cultivated rice, a single individual was used for genome sequencing on the Illumina Hiseq 2500. Library construction and sample indexing were performed as described previously (Huang et al., 2009). The libraries were loaded into the Illumina Hiseq 2500 for 100 bp paired-end sequencing. Image analysis and base calling were conducted using the Illumina Genome Analyzer processing pipeline (v1.4). PERL scripts in the SEG-Map pipeline were used to sort raw sequences on the basis of the 5′ indexes.

A total of 6.773×10⁹ paired-end 100-bp reads were obtained for the cultivated accessions. Firstly, quality control was performed, and the average Q30 was 89.94%, which means that the reads were reliable. Then the reads were aligned to Os-Nipponbare-Reference-MSU7.0 pseudomolecules using bwa-mem with the -M option of BWA software (Li and Durbin, 2010). The mapped reads were realigned using RealignerTargetCreator and indelRealigner of GATK software (DePristo et al., 2011). To label SNPs, UnifiedGenotyper of GATK was used with the -glm BOTH option. All nucleotide polymorphisms were analyzed according to their location in the reference genome.

### Population Genetic Analyses

The population structure of the 102 indica varieties (PCA) was estimated using the software PLINK version 1.9 (http://pngu.mgh.harvard.edu/-purcell/plink/). The LD between SNPs in the 102 varieties was evaluated using squared Pearson's correlation coefficient (r²) as calculated with the -r² command in the software PLINK version 1.9. The LD heatmaps surrounding peaks in the GWAS were constructed using the R package “LD heatmap” (Shin et al., 2006). We estimated the candidate regions using an r²>0.6 (Yano et al., 2016).

Genome wide association study (GWAS)

The population structure (Q) was inferred using Admixture (Alexander et al., 2009), and the best one was selected when cross-validation (CV) errors was minimum. The relative kinship matrix (K) of the natural population was calculated using TASSEL 5.2.1 (Bradbury et al., 2007). GWAS was performed using the Q+K model in TASSEL 5.2.1. The genome-wide significance threshold was determined using permutation-based false-discovery-rate-adjusted P values (Dudbridge and Gusnanto, 2008). The permutation tests were repeated 1,000 times.

### Plasmid Construction and Plant Transformation

The 7897-bp GSE5 genomic sequence was amplified from the indica variety 93-11 using the primers gGUS-F/R and gGFP-F/R and cloned into the pMDC164 and pMDC107 vectors using in-fusion enzyme (Genebank Biosciences Inc, China), respectively. The coding sequences of GSE5 and GSE5L1 were amplified by the specific primers cGSE5-F/R and cGSE5L1-F/R and cloned into the plpkb003 vector using in-fusion enzyme (Genebank Biosciences Inc, China) to generate proActin:GSE5 and proActin:GSE5L1 plasmids, respectively. The 488-bp sequence was amplified from the PCR products of crGSE5-1 and crGSE5-2 using the primers crGSE5-1F and crGSE5-2R and cloned into the vector pMDC99-Cas9 using in-fusion enzyme (Genebank Biosciences Inc, China) to generate the CRISPR/Cas9-GSE5 plasmid. The plasmids were introduced into Agrobaterium tumefaciens strain GV3101 by electroporation, and rice transformation was transformed according to a previous published method (Hiei et al., 1994).

### GUS Staining and GFP Fluorescence Observations

The developing panicles of proGSE5:GSE5-GUS transgenic plants were stained in a GUS buffer according to the method described previously (Wang et al., 2016). The roots of proGSE5:GSE5-GFP transgenic plants were used to investigate the subcellular localization of GSE5. Plasma membrane were stained using FM4-64 (5 μg/ml), and samples were observed using Zeiss LSM 710 NLO confocal microscopy.

### The Bimolecular Fluorescence Complementation (BiFC) Assay

The coding sequence of GSE5 were amplified by specific primers ycGSE5-F/R, fused with the C-terminal fragment of YFP (cYFP), and then subcloned into the pGWB414 vector (Invitrogen) using in-fusion enzyme (Genebank Biosciences Inc, China). The N-terminal fragment of YFP (nYFP) was amplified from pSY736 using the primers YN-736-F and YN-736-R, fused with the OsCaM1-1 gene, and then subcloned into the pGWB414 vector (Invitrogen) using in-fusion enzyme (Genebank Biosciences Inc, China). nYFP-OsCaM1-1 and cYFP-GSE5 constructs were transformed into Agrobacterium strains GV3101. Transient expression of nYFP-OsCaM1-1 and cYFP-GSE5 in Nicotiana benthamiana leaves and fluorescence observation were conducted as described previously (Wang et al., 2016).

### RT-PCR and Quantitative Real-Time PCR

Developing panicles were used to extract total RNA using an RNAprep pure Plant Kit (TIANGEN, China). Total RNA was used for cDNA synthesis with SuperScript III Reverse Transcriptase (Invitrogen). A Lightcycler 480 machine (Roche) was used to conduct quantitative real-time PCR. Relative amounts of qSW5 and GSE5 were calculated using the comparative threshold (Wang et al., 2016). The primers for quantitative real-time RT-PCR are shown in Supplementary Table 4.

### Real-Time Detection of Promoter Activation

The promoter sequences of 6320-bp, 5310-bp and 4547-bp were amplified from indica variety 93-11 genomic DNA using the specific primers of pLUCL-F/R, pLUCM-F/R and pLUCS-F/R and constructed into the vector pGreenII0800-LUC (Hellens et al., 2005) to generate proGSE5:LUC, proGSE5^{DEL1}:LUC and proGSE5^{DEL2}:LUC plasmids, respectively. For proGSE5^{DEL1+IN1}:LUC construction, the 5677-bp PCR fragment was amplified from indica variety Zhefu802 using the specific primers pLUCM-F/R and cloned into the vector pGreenII0800-LUC using in-fusion enzyme (Genebank Biosciences Inc, China). The plasmids were transferred into the Agrobaterium tumefaciens strain GV3101 by electroporation and coinfiltrated into Nicotiana benthamiana leaves. The Firefly and Renilla luciferase activities were measured using a Dual-Luciferase® Reporter Assay System (Promega).

### Phylogenetic Tree Analysis

To analyse the evolutionary history, the approximate 8.4 kb genomic fragments including 6320-bp 5′ flanking sequence, the GSE5 gene and 1580-bp 3′ flanking sequence from 63 cultivated rice and 26 wild rice (O. rufipogon) were amplified and sequenced. The DNA sequences were aligned using the CLUSTAL X 2.1 program. The evolutionary history was inferred using the neighbour-joining method with the MEGA7.0 program.

### Example II; gse-5-Like CRISPR

### Methods:

### Plasmid Construction and Plant Transformation (for GSE5-Like-Crispr)

The 488-bp sequence was amplified from the PCR products of crGSE5L-1 and crGSE5L-2 using the primers crGSE5L-1F and crGSE5L-2R and cloned into the vector pMDC99-Cas9 using in-fusion enzyme (Genebank Biosciences Inc, China) to generate the CRISPR/Cas9-GSE5L plasmid. The plasmids were introduced into Agrobaterium tumefaciens strain GV3101 by electroporation, and rice transformation was transformed according to a previous published method (Hiei et al., 1994).

crGSE5L-1:
(SEQ ID NO: 85)
F: gacggccagtgccaagcttCTCGGATCCACTAGTAACGGC
(SEQ ID NO: 86)
R: CTTCCTGTCCGGCGGGGGCGACACAAGCGACAGCGCGCGGG;
crGSE5L-2:
(SEQ ID NO: 87)
F: CGCCCCCGCCGGACAGGAAGGTTTTAGAGCTAGAAATAGCA
(SEQ ID NO: 88)
R: cctgcaggcatgcaagcttCGACCTCGAGCGGCCGCCAGT

Field-grown plants were raised during the standard rice season at Experimental Stations of the Institute of Genetics and Developmental Biology in Beijing. The spacing between plants was 20 cm.

Grain size of the Zhonghua 11 and GSE5-Like-crispr were measured using the SC Detection and Analysis System of Rice Seeds (Hangzhou WSeen Detection Technology). Actual yield of Zhonghua 11, GSE5-cr and proActin:GSE5 were weighted using electronic analytical balance (METTLER MOLEDO AL104 CHINA).

### Results:

To evaluate the application potential of GSE5 for improving grain yield, we investigated yield traits of Zhonghua 11, GSE5-cr and proActin:GSE5 plants. Actual yield per plant in GSE5-cr was increased compared with that in Zhonghua 11 (FIG. 12A). In rice, LOC_Os01g09470 (here named GSE5-Like) shares significant similarity with GSE5 (72.5% identity). Knocking out GSE5-Like in Zhonghua 11 via CRISPR/Cas9 resulted in significantly increased grain length and width (FIG. 12B-D). Thus GSE5-Like also regulates grain width in rice.

### REFERENCES

Abel, S., Savchenko, T., and Levy, M. (2005). Genome-wide comparative analysis of the IQD gene families in Arabidopsis thaliana and Oryza sativa. BMC Evol. Biol. 5:72.
Alexander, D. H., Novembre, J., and Lange, K. (2009). Fast model-based estimation of ancestry in unrelated individuals. Genome Res. 19:1655-1664.
Bradbury, P. J., Zhang, Z., Kroon, D. E., Casstevens, T. M., Ramdoss, Y., and Buckler, E. S. (2007). TASSEL: software for association mapping of complex traits in diverse samples. Bioinformatics 23:2633-2635.
Cermak, T. et al. Efficient design and assembly of custom TALEN and other TAL effector-based constructs for DNA targeting. Nucleic Acids Res. 39 (2011).
Che, R., Tong, H., Shi, B., Liu, Y, Fang, S., Liu, D., Xiao, Y., Hu, B., Liu, L., Wang, H., et al. (2016). Control of grain size and rice yield by GL2-mediated brassinosteroid responses. Nat. Plants 2:1.
Clough, S. J. and Bent, A. F. (1998), Floral dip: a simplified method for Agrobacterium-mediated transformation of Arabidopsis thaliana. The Plant Journal, 16: 735-743. doi:10.1046/j.1365-313x.1998.00343.x
DePristo, M. A., Banks, E., Poplin, R., Garimella, K. V., Maguire, J. R., Hartl, C., Philippakis, A. A., del Angel, G., Rivas, M. A., Hanna, M., et al. (2011). A framework for variation discovery and genotyping using next-generation DNA sequencing data. Nat. Genet. 43:491-498.
Duan, P., Ni, S., Wang, J., Zhang, B., Xu, R., Wang, Y., Chen, H., Zhu, X., and Li, Y. (2015). Regulation of OsGRF4 by OsmiR396 controls grain size and yield in rice. Nat. Plants 2:1.
Dudbridge, F., and Gusnanto, A. (2008). Estimation of significance thresholds for genomewide association scans. Genet. Epidemiol. 32:227-234.
Fan, C., Xing, Y., Mao, H., Lu, T., Han, B., Xu, C., Li, X., and Zhang, Q. (2006). GS3, a major QTL for grain length and weight and minor QTL for grain width and thickness in rice, encodes a putative transmembrane protein. Theor. Appl. Genet. 112:1164-1171.
Hellens, R. P., Allan, A. C., Friel, E. N., Bolitho, K., Grafton, K., Templeton, M. D., Karunairetnam, S., Gleave, A. P., and Laing, W. A. (2005). Transient expression vectors for functional genomics, quantification of promoter activity and RNA silencing in plants. Plant methods 1:13.
Hiei, Y., Ohta, S., Komari, T., and Kumashiro, T. (1994). Efficient transformation of rice (Oryza sativa L.) mediated by Agrobacterium and sequence analysis of the boundaries of the T-DNA. Plant J. 6:271-282.
Hu, J., Wang, Y., Fang, Y, Zeng, L., Xu, J., Yu, H., Shi, Z., Pan, J., Zhang, D., Kang, S., et al. (2015). A Rare Allele of GS2 Enhances Grain Size and Grain Yield in Rice. Mol. Plant 8:1455-1465.
Huang, X., Feng, Q., Qian, Q., Zhao, Q., Wang, L., Wang, A., Guan, J., Fan, D., Weng, Q., Huang, T., et al. (2009). High-throughput genotyping by whole-genome resequencing. Genome Res. 19:1068-1076.
Huang, X., Wei, X., Sang, T., Zhao, Q., Feng, Q., Zhao, Y., Li, C., Zhu, C., Lu, T., Zhang, Z., et al. (2010). Genome-wide association studies of 14 agronomic traits in rice landraces. Nat. Genet. 42:961-967.
International Rice Genome Sequencing, P. (2005). The map-based sequence of the rice genome. Nature 436:793-800.
Ishimaru, K., Hirotsu, N., Madoka, Y., Murakami, N., Hara, N., Onodera, H., Kashiwagi, T., Ujiie, K., Shimizu, B., Onishi, A., et al. (2013). Loss of function of the IAA-glucose hydrolase gene TGW6 enhances rice grain weight and increases yield. Nat. Genet. 45:707-711.
Li, H., and Durbin, R. (2010). Fast and accurate long-read alignment with Burrows-Wheeler transform. Bioinformatics 26:589-595.
Li, N., and Li, Y. (2016). Signaling pathways of seed size control in plants. Curr. Opin. Plant Biol. 33:23-32.
Li, Y., Fan, C., Xing, Y., Jiang, Y., Luo, L., Sun, L., Shao, D., Xu, C., Li, X., Xiao, J., et al. (2011). Natural variation in GS5 plays an important role in regulating grain size and yield in rice. Nat. Genet. 43:1266-1269.
Mao, H., Sun, S., Yao, J., Wang, C., Yu, S., Xu, C., Li, X., and Zhang, Q. (2010). Linking differential domain functions of the GS3 protein to natural variation of grain size in rice. Proc. Natl. Acad. Sci. USA 107:19579-19584.
Neville E Sanjana, Le Cong, Yang Zhou, Margaret M Cunniff, Guoping Feng & Feng Zhang A transcription activator-like effector toolbox for genome engineering, Nature Protocols 7, 171-192 (2012).
Qi, P., Lin, Y. S., Song, X. J., Shen, J. B., Huang, W., Shan, J. X., Zhu, M. Z., Jiang, L., Gao, J. P., and Lin, H. X. (2012). The novel quantitative trait locus GL3.1 controls rice grain size and yield by regulating Cyclin-T1;3. Cell Res. 22:1666-1680.
Meghdad Randar, Moira A. McMahon, Thazha P. Prakash, Eric E. Swayze, C. Frank Bennett and Don W. Cleveland, Synthetic CRISPR RNA-Cas9-guided genome editing in human cells PNAS 2015 112 (51) E7110-E7117; published ahead of print Nov. 16, 2015, doi:10.1073/pnas.1520883112
Shin, J.-H., Blay, S., McNeney, B., and Graham, J. (2006). LDheatmap: An R Function for Graphical Display of Pairwise Linkage Disequilibria Between Single Nucleotide Polymorphisms. J. Stat. Softw. 16, Code Snippet 3
Shomura, A., Izawa, T., Ebana, K., Ebitani, T., Kanegae, H., Konishi, S., and Yano, M. (2008). Deletion in a gene associated with grain size increased yields during rice domestication. Nat. Genet. 40:1023-1028.
Si, L., Chen, J., Huang, X., Gong, H., Luo, J., Hou, Q., Zhou, T., Lu, T., Zhu, J., Shangguan, Y., et al. (2016). OsSPL13 controls grain size in cultivated rice. Nat. Genet. 48:447-456.
Song, X. J., Huang, W., Shi, M., Zhu, M. Z., and Lin, H. X. (2007). A QTL for rice grain width and weight encodes a previously unknown RING-type E3 ubiquitin ligase. Nat. Genet. 39:623-630.
Wang, S., Li, S., Liu, Q., Wu, K., Zhang, J., Wang, Y, Chen, X., Zhang, Y., Gao, C., Wang, F., et al. (2015a). The OsSPL16-GW7 regulatory module determines grain shape and simultaneously improves rice yield and grain quality. Nat. Genet. 47:949-954.
Wang, S., Wu, K., Yuan, Q., Liu, X., Liu, Z., Lin, X., Zeng, R., Zhu, H., Dong, G., Qian, Q., et al. (2012). Control of grain size, shape and quality by OsSPL16 in rice. Nat. Genet. 44:950-954.
Wang, Y., Xiong, G., Hu, J., Jiang, L., Yu, H., Xu, J., Fang, Y, Zeng, L., Xu, E., Ye, W., et al. (2015b). Copy number variation at the GL7 locus contributes to grain size diversity in rice. Nat. Genet. 47:944-948.
Wang, Z., Li, N., Jiang, S., Gonzalez, N., Huang, X., Wang, Y., Inze, D., and Li, Y. (2016). SCF^{SAP} controls organ size by targeting PPD proteins for degradation in Arabidopsis thaliana. Nat. Commun. 7:11192.
Weng, J., Gu, S., Wan, X., Gao, H., Guo, T., Su, N., Lei, C., Zhang, X., Cheng, Z., Guo, X., et al. (2008). Isolation and initial characterization of GWS, a major QTL associated with rice grain width and weight. Cell Res. 18:1199-1209.
Xia, T., Li, N., Dumenil, J., Li, J., Kamenski, A., Bevan, M. W., Gao, F., and Li, Y. (2013). The Ubiquitin Receptor DA1 Interacts with the E3 Ubiquitin Ligase DA2 to Regulate Seed and Organ Size in Arabidopsis. Plant Cell 25:3347-3359.
Xiao, H., Jiang, N., Schaffner, E., Stockinger, E. J., and van der Knaap, E. (2008). A retrotransposon-mediated gene duplication underlies morphological variation of tomato fruit. Science 319:1527-1530.
Yano, K., Yamamoto, E., Aya, K., Takeuchi, H., Lo, P. C., Hu, L., Yamasaki, M., Yoshida, S., Kitano, H., Hirano, K., et al. (2016). Genome-wide association study using whole-genome sequencing rapidly identifies new genes influencing agronomic traits in rice. Nat. Genet. 48:927-934.
Zhang, X., Wang, J., Huang, J., Lan, H., Wang, C., Yin, C., Wu, Y., Tang, H., Qian, Q., Li, J., et al. (2012). Rare allele of OsPPKL1 associated with grain length causes extra-large grain and a significant yield increase in rice. Proc. Natl. Acad. Sci. USA 109:21534-21539.
Zheng, X. M., and Ge, S. (2010). Ecological divergence in the presence of gene flow in two closely related Oryza species (Oryza rufipogon and O. nivara). Mol. Ecol. 19:2439-2454.
Zhu, Q., Zheng, X., Luo, J., Gaut, B. S., and Ge, S. (2007). Multilocus analysis of nucleotide variation of Oryza sativa and its wild relatives: severe bottleneck during domestication of rice. Mol. Biol. Evol. 24:875-888.
Zuo, J., and Li, J. (2014). Molecular genetic dissection of quantitative trait loci regulating rice grain size. Annu. Rev. Genet. 48:99-118.

SEQUENCE LISTING:
SEQ ID NO: 1. Oryza sativa GSE5 amino acid
MGKAARWFRNMWGGGRKEQKGEAPASGGKRWSFGKSSRDSAEAAAAAAAAAAEA
SGGNAAIARAAEAAWLRSVYADTEREQSKHAIAVAAATAAAADAAVAAAQAAVAVVR
LTSKGRSAPVLAATVAGDTRSLAAAAVRIQTAFRGFLAKKALRALKALVKLQALVRGYL
VRRQAAATLQSMQALVRAQATVRAHRSGAGAAANLPHLHHAPFWPRRSLQERCAG
DDTRSEHGVAAYSRRLSASIESSSYGYDRSPKIVEVDTGRPKSRSSSSRRASSPLLLD
AAGCASGGEDWCANSMSSPLPCYLPGGAPPPRIAVPTSRHFPDYDWCALEKARPAT
AQSTPRYAHAPPTPTKSVCGGGGGGGIHSSPLNCPNYMSNTQSFEAKVRSQSAPKQ
RPETGGAGAGGGRKRVPLSEVVVVESRASLSGVGMQRSCNRVQEAFNFKTAVVGR
LDRSSESGENDRHAFLQRRW
SEQ ID NO: 2: Oryza sativa GSE5 nucleic acid (CDS)
ATGGGCAAGGCGGCGCGGTGGTTCCGCAACATGTGGGGAGGAGGGAGGAAGGA
GCAGAAGGGCGAGGCGCCGGCGAGTGGGGGGAAGAGGTGGAGCTTCGGGAAG
TCGTCGAGGGACTCGGCGGAGGCCGCGGCGGCTGCTGCTGCGGCGGCGGCGG
AGGCTTCCGGGGGCAATGCGGCGATCGCCAGGGCGGCCGAGGCGGCGTGGCT
CAGGTCGGTGTACGCCGACACGGAGCGGGAGCAGAGCAAGCACGCCATCGCCG
TCGCCGCGGCCACCGCGGCGGCGGCTGATGCCGCCGTGGCGGCCGCTCAGGC
CGCCGTCGCCGTCGTGCGGCTTACTAGCAAGGGCCGCTCGGCTCCCGTCCTCGC
CGCCACCGTCGCCGGCGACACGCGCAGCCTTGCCGCCGCCGCCGTCAGAATCC
AGACGGCATTCAGAGGCTTCCTGGCGAAGAAGGCGCTGCGAGCGCTCAAGGCG
CTGGTGAAGCTGCAGGCGCTGGTGCGCGGCTACCTCGTTCGCCGGCAGGCCGC
CGCCACGCTGCAGAGCATGCAGGCGCTCGTCCGCGCCCAGGCCACTGTCCGCG
CCCACCGCAGTGGCGCCGGCGCCGCCGCCAATCTCCCGCACCTCCACCACGCT
CCCTTCTGGCCCCGCCGCTCGCTGCAGGAGAGGTGCGCCGGCGACGACACGAG
GAGCGAGCACGGTGTGGCGGCGTACAGCCGGCGGCTGTCGGCGAGCATCGAGT
CGTCGTCGTACGGGTACGACCGGAGCCCCAAGATCGTGGAGGTGGACACCGGG
AGGCCCAAGTCGCGGTCGTCGTCGTCGCGGCGGGCGAGCTCCCCGCTGCTGCT
CGACGCCGCTGGGTGCGCGAGCGGCGGCGAGGACTGGTGCGCCAACTCCATGT
CGTCGCCGCTCCCGTGCTACCTCCCCGGCGGCGCGCCGCCGCCCCGCATCGCC
GTCCCGACGTCGCGCCACTTCCCCGACTACGACTGGTGCGCGCTGGAGAAGGCC
CGGCCGGCGACGGCGCAGAGCACGCCGCGGTACGCGCACGCGCCGCCGACGC
CGACCAAGAGCGTGTGCGGCGGCGGCGGCGGCGGCGGCATCCACTCGTCGCC
GCTCAACTGCCCGAACTACATGTCCAACACGCAGTCGTTCGAGGCGAAGGTGCG
TTCGCAGAGCGCGCCGAAGCAGCGGCCGGAGACCGGCGGCGCCGGCGCCGGC
GGCGGCCGGAAGCGGGTGCCGCTGAGCGAGGTGGTGGTGGTGGAGTCCAGGG
CGAGCTTGAGCGGCGTGGGCATGCAGCGCTCGTGCAACCGGGTGCAGGAGGCG
TTCAACTTCAAGACGGCCGTCGTCGGCCGCCTCGACCGCTCGTCGGAGTCCGGC
GAGAACGACCGCCACGCGTTCTTGCAGAGGAGGTGGTGA
SEQ ID NO: 3: Triticum aestivum GSE5 amino acid
MGKAARWLRGLLGGGGKKEQGKEQRRPATAPHGDRKRWSFCKSTRDSAEAEAAAA
AAALSGNAAIARAAEAAWLKSLYNETEREQSKHAIAVAAATAAAADAAMAAAQAAVEV
VRLTSKGPTSTVLADAVAEPHGRASAAVKIQTAFRGFLAKKALRALKGLVKLQALVRG
YLVRKQAAATLQSMQALVRAQACIRAARSRAAALPTNLRVHPTPVRPRYSLQERYST
TEDSRSDHRVAPYYSRRLSASVESSSCYGYDRSPKIVEMDTGRPKSRSSSLRTTSPG
ASEECYAHSVSSPLMPCRAPPRIAAPTARHFPEYEWCEKARPATAQSTPRYTSYAPV
TPTKSVCGGYTYSNSPSTLNCPSYMSSTQSSVAKVRSQSAPKQRPEEGAVRKRVPL
SEVIILQEARASLGGGGGTQRSCNRPAQEEAFSFKKAVVSRFDRSSEAAERERDRDR
DLFLQKGW
SEQ ID NO: 4: Triticum aestivum GSE5 nucleic acid (CDS)
ATGGGCAAGGCGGCGAGGTGGCTGCGTGGCTTGCTGGGCGGCGGCGGCAAGAA
GGAGCAGGGGAAGGAGCAGAGGCGCCCGGCCACGGCGCCGCACGGGGACAGG
AAGCGCTGGAGCTTCTGCAAGTCCACCAGGGACTCGGCAGAGGCGGAGGCGGC
GGCCGCGGCCGCGGCGCTCAGCGGCAACGCGGCGATCGCGCGCGCGGCCGAG
GCGGCATGGCTCAAGTCCTTGTACAACGAGACCGAGCGCGAGCAGAGCAAGCAC
GCCATCGCCGTCGCCGCGGCCACCGCGGCGGCGGCGGACGCGGCTATGGCTG
CCGCACAGGCAGCCGTGGAGGTCGTGCGGCTCACCAGCAAAGGGCCGACGTCG
ACGGTGCTCGCCGACGCCGTCGCGGAGCCCCACGGCCGTGCCTCCGCCGCGGT
CAAGATCCAGACGGCGTTCCGTGGCTTCCTGGCCAAGAAGGCTCTGCGCGCGCT
CAAGGGGCTGGTGAAGCTGCAGGCGCTGGTGCGCGGCTACCTGGTGCGGAAGC
AGGCGGCGGCCACGCTGCAGAGCATGCAGGCGCTCGTCCGCGCGCAGGCCTGC
ATCCGCGCTGCCCGCTCGCGCGCCGCGGCGCTCCCGACGAACCTTCGCGTCCA
CCCCACTCCTGTCCGGCCGCGCTACTCGTTGCAAGAGCGGTACAGCACCACGGA
GGATTCCCGGAGCGACCACCGCGTGGCGCCGTACTACAGCCGCCGGCTGTCGG
CGAGCGTGGAGTCGTCGTCGTGCTACGGCTACGACCGGAGCCCCAAGATCGTGG
AGATGGACACCGGCCGGCCCAAGTCGCGCTCCTCCTCGCTCCGGACGACCTCCC
CCGGCGCCAGCGAGGAGTGCTACGCCCACTCGGTGTCGTCGCCGCTCATGCCG
TGCCGAGCGCCCCCGCGGATCGCGGCGCCCACCGCGCGCCACTTCCCGGAGTA
CGAGTGGTGCGAGAAGGCCCGGCCGGCGACGGCGCAGAGCACGCCCCGGTACA
CGAGCTACGCGCCGGTGACGCCGACCAAGAGCGTGTGCGGCGGCTACACCTAC
AGCAACAGCCCGTCGACGCTCAACTGCCCCAGCTACATGTCGAGCACGCAGTCG
TCCGTGGCGAAGGTGCGTTCGCAGAGCGCGCCGAAGCAGCGGCCGGAGGAGGG
CGCGGTACGGAAGAGGGTGCCGCTGAGCGAGGTGATCATCCTGCAGGAGGCCC
GGGCGAGCCTGGGCGGCGGCGGGGGCACGCAGAGGTCGTGCAACCGGCCGGC
GCAGGAGGAGGCGTTCAGCTTCAAGAAGGCCGTCGTGAGCCGCTTCGACCGCTC
GTCGGAGGCGGCCGAGAGGGAACGTGACCGGGACCGGGACTTGTTCCTGCAGA
AGGGGTGGTGA
SEQ ID NO: 5: Zea mays GSE5 amino acid
MGKAARWFRSFLGKKEQRPTKDQRRLQQQDDQAPPLPPPSAKRWSFGRSSRDSAA
AAVVSAGAGNAAIARAAEAAWLRSAACAETHRDRDQDQDQSKHAIAVAAATAAAADA
AVAAAQAAVAVVRLTSKGRAPLFAVAAAVRIQTAFRGFLCSVAALPRCVPSTQAKKAL
RALKALVKLQALVRGYLVRRQAAATLQSMQALVRAQATVRARRAGAAALPHLHHLPG
RPRYSMQERCADDARIEHGVAAHSSRRLSASVESSSYGYDRSPKIVEVDPGRPKSRS
SSRRSSAPLLDAGSCCGEEWCASANPASSPLPCYLSAGPPTRIAVPTSRQFPDYDW
CALEKARPATAQSTPRCLLQAHAPATPTKSVVAGHSPSLNGCPNYMSSTQASEAKAR
SQSAPKQRPELACCCGGARKRVPLSEVVLVDSSRASLSGVVGMQRGCSTGAQEAFS
FRTAVVGRIDRSLEVAGGENDRLALLQRRW
SEQ ID NO: 6: Zea mays GSE5 nucleic acid (CDS)
ATGGGCAAGGCGGCGCGCTGGTTCCGCAGCTTCCTGGGCAAGAAAGAGCAGCG
GCCCACCAAGGACCAGCGGCGGCTGCAGCAGCAGGACGACCAGGCTCCTCCGC
TTCCGCCGCCAAGCGCCAAGCGCTGGAGCTTCGGTAGGTCGTCGCGGGACTCG
GCGGCGGCCGCGGTCGTCTCGGCCGGCGCGGGCAACGCGGCGATCGCGCGCG
CCGCCGAGGCCGCGTGGCTCAGGTCCGCCGCGTGCGCCGAGACGCACCGGGA
CCGGGACCAGGACCAGGACCAGAGCAAGCACGCCATCGCCGTGGCCGCCGCCA
CCGCCGCCGCGGCGGACGCGGCGGTGGCGGCGGCGCAGGCGGCAGTCGCCGT
TGTGCGCCTCACCAGCAAGGGACGCGCGCCGCTCTTCGCCGTCGCCGCCGCCG
TCAGGATCCAGACGGCGTTCCGAGGATTCTTGTGTTCTGTTGCTGCCCTGCCGCG
GTGTGTTCCTTCTACGCAGGCCAAGAAGGCGTTGCGCGCGCTCAAGGCGCTCGT
GAAGCTGCAGGCGCTGGTGCGCGGCTACCTCGTGCGCAGGCAGGCGGCCGCCA
CGCTGCAGAGCATGCAGGCTCTCGTCCGCGCGCAGGCCACCGTGCGCGCGAGA
CGAGCCGGCGCCGCCGCCCTCCCGCACCTCCACCACCTGCCCGGCCGCCCGCG
CTACTCGATGCAAGAGCGGTGCGCGGACGACGCGCGGATCGAGCACGGGGTGG
CGGCGCACAGCAGCCGGCGGCTGTCGGCGAGCGTGGAGTCCTCGTCGTACGGC
TACGACCGGAGTCCCAAGATCGTGGAGGTGGACCCCGGCCGCCCCAAGTCGCG
GTCGTCCTCGCGCCGCTCGAGCGCCCCGCTGCTCGACGCCGGCAGCTGCTGCG
GCGAGGAGTGGTGCGCCAGCGCCAACCCCGCGTCCTCGCCGCTGCCGTGCTAC
CTGTCCGCCGGGCCGCCGACGCGCATCGCCGTGCCGACCTCGCGCCAGTTCCC
GGACTACGACTGGTGCGCGCTGGAGAAGGCCCGGCCGGCCACGGCGCAGAGCA
CGCCGCGGTGCCTGCTGCAGGCGCACGCGCCGGCCACCCCGACCAAGTCCGTC
GTCGCGGGCCACTCGCCGTCGCTTAACGGGTGCCCGAACTACATGTCGAGCACG
CAGGCGTCGGAGGCCAAGGCGCGGTCTCAGAGCGCGCCGAAGCAGCGGCCCGA
GCTCGCCTGCTGCTGCGGCGGAGCGCGCAAGCGGGTGCCGCTCAGCGAGGTGG
TTCTCGTGGATTCCTCCCGCGCCAGCCTGAGCGGCGTCGTGGGCATGCAGCGCG
GGTGCAGCACCGGGGCGCAGGAGGCGTTCAGCTTCCGGACGGCCGTCGTTGGT
CGCATAGACCGCTCGTTGGAGGTTGCCGGCGGCGAGAACGACCGGCTGGCCTT
GTTGCAGAGGAGGTGGTGA
SEQ ID NO: 7: Glycine max GSE5 amino acid
MGRATRWVKSLFGIRREKEKKLNFRCGEAKSMELCCSESTSNSTVLCHNSGTIPPNL
SQAEAAWLQSFCTEKEQNKHAIAVAAATAAAADAAVAAAQAAVAVVRLTSQGRGRT
MFGVGPEMWAAIKIQTVFRGFLARKALRALKGLVKLQALVRGYLVRKLATATLHSMQA
LVRAQARMRSHKSLRPMTTKNEAYKPHNRARRSMERFDDTKSECAVPIHSRRVSSS
FDATINNSVDGSPKIVEVDTFRPKSRSRRAISDFGDEPSLEALSSPLPVPYRTPTRLSIP
DQRNIQDSEWGLTGEECRFSTAHSTPRFTNSCTCGSVAPLTPKSVCTDNYLFLRQYG
NFPNYMTSTQSFKAKLRSHSAPKQRPEPGPRKRISLNEMMESRNSLSGVRMQRSCS
QVQEVINFKNVVMGKLQKST
SEQ ID NO: 8: Glycine max GSE5 nucleic acid (CDS)
ATGGGGAGAGCCACTAGGTGGGTGAAGAGTTTGTTTGGAATAAGAAGAGAGAAA
GAGAAGAAACTAAACTTCAGGTGTGGAGAGGCTAAAAGTATGGAATTGTGTTGTT
CTGAGAGTACTAGTAATTCAACAGTTTTGTGTCACAATTCAGGGACTATACCCCCC
AACCTTTCTCAAGCTGAGGCTGCTTGGTTACAATCATTCTGCACAGAGAAGGAGC
AAAACAAGCACGCCATCGCAGTTGCTGCTGCCACGGCGGCAGCTGCTGATGCTG
CCGTGGCAGCAGCACAGGCTGCGGTGGCGGTTGTTAGGCTCACCAGCCAAGGAA
GGGGTCGCACCATGTTTGGTGTTGGACCTGAGATGTGGGCTGCCATCAAGATTCA
AACAGTGTTTAGAGGATTCCTGGCAAGGAAGGCACTAAGGGCATTAAAAGGATTG
GTGAAATTGCAGGCACTTGTCAGAGGGTATTTAGTGAGGAAGCTAGCAACAGCAA
CCCTGCATAGTATGCAGGCTCTTGTTAGAGCTCAAGCTAGAATGCGGTCCCACAA
ATCTCTCAGGCCCATGACCACAAAGAATGAAGCATATAAACCTCATAATAGAGCAA
GAAGATCCATGGAGAGGTTTGATGACACTAAGAGTGAGTGTGCAGTTCCAATCCA
CAGTAGAAGGGTATCATCTTCTTTTGATGCTACAATTAACAACAGTGTTGATGGGA
GCCCCAAAATAGTGGAAGTGGACACTTTCAGGCCTAAGTCAAGGTCTAGAAGGGC
AATTTCAGATTTTGGTGATGAACCATCACTAGAAGCACTTTCTTCTCCCTTACCAGT
TCCGTACAGAACCCCTACACGTTTGTCCATACCAGACCAAAGGAATATTCAGGACT
CTGAATGGGGGTTAACAGGAGAAGAGTGCAGATTCTCTACAGCACATAGCACTCC
GCGCTTCACAAATTCTTGTACCTGTGGCTCAGTTGCACCATTGACACCAAAGAGT
GTGTGCACTGATAACTACTTGTTCCTAAGGCAGTATGGGAATTTTCCAAACTACAT
GACTAGTACTCAGTCTTTTAAGGCCAAATTGAGGTCTCATAGTGCTCCAAAGCAAC
GGCCAGAACCTGGTCCAAGGAAGAGGATTTCCCTCAATGAAATGATGGAGTCTAG
GAATAGTTTGAGTGGGGTTAGAATGCAGAGGTCTTGCTCACAGGTTCAAGAAGTC
ATTAATTTCAAGAATGTTGTGATGGGGAAGCTTCAGAAATCCACATAA
SEQ ID NO: 9: Sorghum bicolor GSE5 amino acid
MGKAARWFRSFLGGKKEQQATKDHRRRQQQQQQDQPPPPPPPPATTAKRWSFGK
SSRDSAEAAAAVVSAGAGNAAIARAAEAAWLRSAACAETDREREQSKHAIAVAAATA
AAADAAVAAAQAAVAVVRLTNKGRAPPGVLATAGGGRAAAAAVRIQTAFRGFLAKKA
LRALKALVKLQALVRGYLVRRQAAATLQSMQALVRAQAAVRARRAAAAALSQSHLHH
HHHPPPVRPRYSLQERYADDTRSEHGVAAYSSRRLSASVESSSYGGYDRSPKIVEVD
PGRPKSRSSSSRRASSPLLDAAGGSSGGEDWCAANPASSSPLPCYLSAAGGPPRIA
VPTSRQFPDYDWCALEKARPATAQSTPRYLLPATPTKSVAGNSPSLHGCPNYMSST
QASEAKVRSQSAPKQRPELACCAGGGGGGARKRVPLSEVVVVESSRASLSGVVGM
QRGCGGARAQEAFSFRAAVVGRMDRSLEVAGIENDRQAFLQRRW
SEQ ID NO: 10: Sorghum biocolor GSE5 nucleic acid (CDS)
ATGGGCAAGGCGGCGCGCTGGTTCCGCAGCTTCCTGGGCGGCAAGAAGGAGCA
GCAGGCCACCAAAGATCACCGGCGGCGCCAGCAGCAGCAGCAGCAGGACCAGC
CTCCTCCTCCTCCGCCTCCGCCGGCCACCACCGCCAAGCGCTGGAGCTTCGGCA
AGTCGTCGCGGGACTCGGCCGAGGCGGCCGCGGCCGTCGTCTCGGCCGGCGC
GGGCAACGCGGCGATCGCGCGCGCCGCGGAGGCCGCCTGGCTCAGGTCCGCC
GCGTGCGCCGAGACGGACCGCGAGCGGGAGCAGAGCAAGCACGCCATCGCCGT
GGCCGCCGCCACCGCCGCCGCGGCCGACGCGGCGGTCGCCGCGGCGCAGGCG
GCCGTCGCCGTCGTCCGACTCACAAACAAGGGACGCGCGCCGCCCGGCGTCCT
CGCCACCGCTGGAGGAGGACGCGCCGCCGCCGCCGCCGTCAGGATCCAGACGG
CGTTCCGAGGATTCTTGGCGAAGAAGGCGTTGCGCGCGCTCAAGGCGCTCGTGA
AGCTGCAGGCGCTGGTGCGCGGCTACCTCGTGCGCAGGCAGGCGGCCGCCACG
CTGCAGAGCATGCAGGCGCTCGTCCGCGCGCAGGCCGCCGTGCGCGCCAGGCG
CGCCGCCGCCGCCGCGCTCTCGCAGTCGCACCTCCACCACCACCACCACCCGC
CGCCCGTCCGTCCGCGCTACTCGCTGCAAGAGCGGTACGCGGACGACACGCGG
AGCGAGCACGGGGTGGCGGCGTACAGCAGCCGGCGGCTGTCGGCGAGCGTCG
AGTCCTCGTCGTACGGCGGCTACGACCGGAGCCCCAAGATCGTGGAGGTGGACC
CGGGCCGCCCCAAGTCGCGCTCGTCGTCCTCGCGCCGGGCCAGCTCACCGCTG
CTCGACGCCGCCGGCGGCAGCAGCGGCGGCGAGGACTGGTGCGCCGCCAACC
CCGCGTCGTCGTCGCCGCTGCCGTGCTACCTGTCCGCCGCCGGCGGACCGCCG
CGCATCGCCGTGCCGACCTCGCGCCAGTTCCCGGACTACGACTGGTGCGCGCTC
GAGAAGGCCCGCCCGGCCACGGCGCAGAGCACGCCGCGGTACCTGCTGCCGGC
CACCCCGACGAAGTCCGTCGCGGGAAACTCGCCGTCGCTGCACGGGTGCCCGA
ACTACATGTCGAGCACGCAGGCGTCGGAGGCCAAGGTGCGGTCCCAGAGCGCG
CCCAAGCAGCGGCCCGAGCTCGCCTGCTGCGCCGGCGGCGGCGGCGGGGGAG
CGCGGAAGCGGGTGCCGCTCAGCGAGGTGGTGGTCGTGGAGTCGTCCCGCGCC
AGCCTGAGCGGCGTCGTGGGCATGCAGCGCGGGTGCGGCGGCGCCCGGGCGC
AGGAGGCGTTCAGCTTCAGGGCAGCCGTCGTTGGCCGCATGGACCGCTCGTTGG
AGGTTGCCGGTATCGAGAACGACCGGCAGGCGTTCTTGCAGAGGAGGTGGTGA
SEQ ID NO: 11: Medicago truncatula GSE5 amino acid
MGRTIRWFKSLFGIKKDRDNSNSNSSSTKWNPSLPHPPSQDFSKRDSRGLCHNPATI
PPNISPAEAAWVQSFYSETEKEQNKHAIAVAALPWAVVRLTSHGRDTMFGGGHQKFA
AVKIQTTFRGYLARKALRALKGLVKLQALVRGYLVRKQATATLHSMQALIRAQATVRS
HKSRGLIISTKNETNNRFQTQARRSTERYNHNESNRNEYTASIPIHSRRLSSSFDATM
NSYDIGSPKIVEVDTGRPKSRSRRSNTSISDFGDDPSFQTLSSPLQVTPSQLYIPNQR
NYNESDWGITGEECRFSTAQSTPRFTSSCSCGFVAPSTPKTICGDSFYIGDYGNYPN
YMANTQSFKAKLRSHSAPKQRPEPGPKKRLSLNELMESRNSLSGVRMQRSCSQIQD
AINFKNAVMSKLDKSTDFDRNFSKQRRL
SEQ ID NO: 12: Medicago truncatula GSE5 nucleic acid (CDS)
ATGGGTAGAACCATAAGGTGGTTCAAGAGTTTGTTTGGGATAAAGAAAGACAGAG
ATAATTCAAACTCAAATTCTTCAAGTACCAAATGGAATCCTTCTCTTCCTCATCCTC
CTTCTCAAGATTTCTCAAAGAGAGATTCGAGAGGCTTGTGTCATAATCCAGCTACC
ATACCTCCCAACATTTCACCTGCAGAAGCTGCTTGGGTTCAATCCTTCTACTCAGA
AACTGAGAAGGAGCAAAACAAGCACGCCATTGCGGTAGCAGCTCTGCCGTGGGC
TGTGGTTAGATTAACCAGCCACGGCAGAGACACCATGTTTGGTGGTGGACACCAG
AAATTTGCTGCTGTCAAGATTCAAACAACATTTAGGGGTTACTTGGCAAGAAAAGC
ACTAAGAGCCTTAAAGGGATTGGTAAAGTTACAAGCACTAGTGAGAGGGTACTTA
GTGAGGAAGCAAGCAACAGCAACATTACACAGTATGCAAGCTCTAATTAGAGCAC
AAGCAACAGTAAGGTCTCATAAATCTCGTGGACTCATCATAAGCACAAAGAATGAA
ACAAATAACAGATTTCAAACACAAGCTAGAAGATCCACGGAAAGGTATAATCACAA
TGAGAGTAACAGGAACGAGTACACAGCTTCAATTCCTATTCACAGCAGAAGATTAT
CATCATCTTTTGATGCTACAATGAACAGTTATGATATTGGAAGTCCAAAAATAGTAG
AAGTTGATACTGGAAGACCAAAATCAAGGTCTAGAAGAAGCAATACATCAATTTCA
GATTTTGGAGATGACCCTTCATTTCAAACACTTTCTTCTCCACTTCAAGTTACTCCA
TCTCAGTTATACATTCCAAATCAAAGAAATTATAACGAATCAGATTGGGGAATAACA
GGTGAAGAATGCAGATTTTCAACTGCACAGAGCACTCCACGTTTCACAAGTTCATG
TAGTTGTGGATTTGTTGCACCTTCCACACCTAAAACAATTTGTGGAGATAGTTTTTA
CATTGGTGATTATGGTAATTATCCTAATTACATGGCTAATACACAGTCTTTTAAGGC
TAAATTGAGGTCTCATAGTGCTCCAAAGCAACGACCTGAACCAGGTCCGAAGAAG
AGGCTTTCATTGAATGAATTGATGGAATCTAGAAACAGTTTGAGTGGAGTTAGAAT
GCAGAGGTCTTGTTCACAGATTCAGGATGCTATTAATTTTAAGAATGCTGTGATGA
GTAAACTTGATAAGTCCACTGATTTTGATAGAAACTTTTCAAAGCAGAGGAGGTTG
TGA
SEQ ID NO: 13: Arabidopsis thaliana GSE5 amino acid
MGRAARWFKGIFGMKKSKEKENCVSGDVGGEAGGSNIHRKVLQADSVWLRTYLAET
DKEQNKHAIAVAAATAAAADAAVAAAQAAVAVVRLTSNGRSGGYSGNAMERWAAVKI
QSVFKGYLARKALRALKGLVKLQALVRGYLVRKRAAETLHSMQALIRAQTSVRSQRIN
RNNMFHPRHSLERLDDSRSEIHSKRISISVEKQSNHNNNAYDETSPKIVEIDTYKTKSR
SKRMNVAVSECGDDFIYQAKDFEWSFPGEKCKFPTAQNTPRFSSSMANNNYYYTPP
SPAKSVCRDACFRPSYPGLMTPSYMANTQSFKAKVRSHSAPRQRPDRKRLSLDEIM
AARSSVSGVRMVQPQPQPQTQTQQQKRSPCSYDHQFRQNETDFRFYN
SEQ ID NO: 14: Arabidopsis thaliana GSE5 nucleic acid (CDS)
ATGGGAAGAGCTGCGAGATGGTTCAAGGGTATTTTTGGTATGAAGAAGAGCAAAG
AGAAAGAGAACTGTGTTTCCGGCGACGTTGGAGGTGAAGCCGGTGGTTCTAACAT
TCACCGGAAAGTTCTCCAAGCTGACTCCGTCTGGCTCAGAACTTACCTTGCGGAA
ACAGACAAAGAACAGAACAAACACGCGATTGCGGTTGCTGCTGCTACAGCCGCG
GCTGCTGACGCAGCGGTTGCAGCGGCTCAAGCTGCTGTGGCGGTGGTCAGGTTA
ACAAGTAACGGAAGAAGCGGAGGATATTCCGGGAACGCAATGGAGCGGTGGGCC
GCAGTGAAAATTCAATCAGTCTTCAAGGGCTATTTGGCGAGAAAAGCGTTACGAG
CTTTGAAAGGTTTAGTGAAGCTACAAGCTTTGGTAAGAGGATACTTAGTCCGCAAA
CGCGCCGCCGAAACGCTGCATAGCATGCAAGCTCTCATTAGAGCTCAAACCAGC
GTCCGATCGCAACGCATCAACCGCAACAACATGTTTCATCCTCGACACTCACTTG
AGAGGTTGGATGATTCAAGAAGTGAAATCCATAGCAAGAGAATATCAATCTCTGTA
GAGAAACAGAGTAATCACAACAACAATGCGTACGATGAGACCAGTCCCAAGATTG
TGGAGATTGATACTTACAAGACGAAATCAAGATCAAAGAGAATGAATGTGGCTGTA
TCCGAATGTGGAGATGATTTCATCTATCAAGCCAAAGATTTCGAATGGAGTTTTCC
GGGAGAGAAATGCAAGTTTCCTACGGCTCAAAACACGCCGAGATTCTCTTCATCA
ATGGCTAATAATAACTATTACTACACGCCCCCATCGCCGGCGAAAAGTGTTTGCA
GAGACGCTTGTTTTAGGCCAAGTTATCCTGGTTTGATGACACCTAGCTATATGGCT
AATACGCAGTCGTTTAAAGCCAAGGTACGTTCGCATAGTGCACCGAGACAACGTC
CTGATAGAAAAAGATTGTCACTTGATGAGATTATGGCGGCTAGAAGTAGCGTTAGT
GGTGTGAGGATGGTGCAACCACAACCACAACCGCAAACGCAAACGCAGCAACAG
AAACGCTCTCCTTGTTCGTATGATCATCAGTTTCGTCAGAACGAGACTGATTTTAG
ATTCTATAATTAG
SEQ ID NO: 15 Oryza sativa target sequence
CGAGGCGGCGTGGCTCAGGTCGG
SEQ ID NO: 16: Triticum aestivum target sequence
CAGCAAAGGGCCGACGTCGACGG
SEQ ID NO: 17: Zea mays target sequence
CCGCGTGCGCCGAGACGCACCGG
SEQ ID NO: 18: Glycine max target sequence
AGGCTGCGGTGGCGGTTGTTAGG
SEQ ID NO: 19: Medicago truncatula target sequence
TTCTCAAAGAGAGATTCGAGAGG
SEQ ID NO: 20: Arabidopsis thaliana target sequence
ACAGAACAAACACGCGATTGCGG
SEQ ID NO: 21: Oryza sativa protospacer sequence
CGAGGCGGCGTGGCTCAGGT
SEQ ID NO: 22: Triticum aestivum protospacer sequence
CAGCAAAGGGCCGACGTCGA
SEQ ID NO: 23: Zea mays protospacer sequence
CCGCGTGCGCCGAGACGCAC
SEQ ID NO: 24: Glycine max protospacer sequence
AGGCTGCGGTGGCGGTTGTT
SEQ ID NO: 25: Medicago truncatula protospacer sequence
TTCTCAAAGAGAGATTCGAG
SEQ ID NO: 26: Sorghum bicolor protospacer sequence
GTCGAGTCCTCGTCGTACGG
SEQ ID NO: 27:
GTTTTAGAGCTAGAAATAGCAAGTTAAAATAAGGCTAGTCCGTTATCAACTTGAAA
AAGTGGCACCGAGTCGGTGC
SEQ ID NO: 28 Oryza sativa GSE5 promoter
ATGTTATACCGCCGTTCTGCCTGTTATTTTGGTGTATGGTGAATGTTATGGAGTAT
TGCTTCATTTTTAAATGAAAGGCACCATCTTAGATTTTTTTTCCTTTAGTTGATTGGT
TGTAGTACTATATTTTCTAAGTTATTATAAAATAAATATGCGATATATATTTGATTGC
CATTATCGAGTTAGTATATCTTCACTCTGTTTATATTTTAAAGCTGTGTACTCCTAG
CTATAGCACTAAAGACCGAACTTAGTATGGACTAATTACAGCGATAACCATCGGTA
ATTGCTACTACTCCATGCAGTACTTGGTTTCCATATTTTTGCATGCGTCGGTCGTT
GGAGGCAAGCAAGCAAGCTGCAGCGTCGTCAGAGGTAGACGCATCGATGTTGTT
CTTCCATGTCCTTCGAATCTTCATGATTGCCGCTCTCTGCTTAATTACTGTTCTCTT
CACGCACACCGTCCAAATCCCATCCATCTATCCATCCGTCCACACACCTGCAAGT
TTAAATTCCCATGCAGTTACTACATGAACAGTACTGCTGTCAAGTTTAATTAATTTC
TACTACACACGAATTGAATTCAAATGGTACTAGGTACAAGTACTCCACTAGTACGA
CCATGATGTTTCCCACCTGAAAGCAACACAGAATACGTTCGTACGTACACGTACA
GTAAACCACAATAATGCACAAGAGTAGGGATCGATGGAGAGTACATGCAATTGCT
CATCACTTGTCACCATCACTGGTTCAAATTTGCCTACTCTAGATCTCGATCGTGGT
ACTGCTTCTAACCATAATGATCGGTACATGTTCAAATTCGAAACTCAAAAAATGTGT
GTACAATACGGCCGGGAGAGATGAACATATATACAGTACTCATAATTATTAACCAT
GCATGGGCATTAATAAAAGTAGTACTCCGTATATAGCTTCCAAGCAAGGCTACATA
CTAGAATCATACTGTATCTTGATTGCCAGAGGTGCAATCATGCATAGACATACTTA
AGATCAGGGCTAGCTAGTTCAGGTGATGAACACATGCTTTGTACCCCTTGTAGGA
TGCATGGCACCAAGTCGACATTGGAGTACACACACCCTGGCCCTGCTGCACGTGT
AGATAAGAGTACCACCTATCATCATCAATCACGCAATATGGTCTACTGTGTCTCTT
TGATTGGGTGTAATATAATATCGGCCACTGTAAAAATTTAAAATTTAAAACTTAGTA
ATTTTAATTTCGAAGTTGATTTTATGATATGCTCAACGTATCTCTTTTTTTTCAAGCT
AGCGTTGGTCTTTAAAAGTGCGTTTGTTTATAAGGTTAGGGTGAGACTTTTAGCTT
GTGTTTGGTTAGTGGGATATGGAATGAGATGGGTTAGGTCGATCCTATTTTTCGAG
CTGTTTGGGTAGAGGGATGTGTAGGACGGGATGATCCTAGATGGGAATATTCTTC
CCAGATCCAGGACGAGGTGGTCCGCCAAAATCGGCTGGACTAATCCATCCCACTT
TGATGGAGCGAATGGCATTAGCTCCTTCCGCCTCCTGCTCGGCTCCGCCACTGG
CCGCCCGTTCCTCCTCCTCCGTCTGCTCCCTCCCACGGCGTCCGTCCCTCCTCCT
CTTCCTGCTCCCTCCCCCGATGCCGCTCCGTCCTCCTCCGCCTGCTCCCTCCCCC
GGCGCCCGTCCCTCCTCCTCTGCCCGCTCGCCAAGTTGCCGGCTGCACCGCCCG
CTCGTGCAGCTCCTCCTGCCATCCGCGCCGCCGGTCGCTGATCGCGCCGCTCCT
CCTTCTGCCTACTCCCTCCCCGGCACCGCTCCTCCTTCTGCCTACTCCCTCCCCC
GGCACCACTCCGTCCTCCTCCGCTTGCTCCCTCCCCCGGCGCCGCTCCCTCCCC
CAGCGCCCGTTAGTCCTCCTCCGCCCGCTCATGCAGCTCCTTCCCTACACACGCT
CGCTCGCCCGCGCCGCTCCTCATTCTGCCGTTCGCCGCTCACGCCTGTGCCTGC
TCGCTTGCACGCCGCTCCTTCCATCCACACCGCCGGTCGCCGGTTGTTTGCCGC
TCCTGCTTCCACCGCCCGCCGTTCGCTGCTCCTTCCTCACTAGGAGTAGACGTGG
TGCTAACATCTCCAAACGTCATTCATCCCATCCCCACCCCTATCCATTCTACCAAA
CAAAAAACTAGCATCATCTCATTTTATAAACCAAACAAGTATGTGAGATCACCCAAT
CCCTAAAATCAGGGATGGTTTCATCCTATCCCACATAGTCCCCAAACAAAACACAT
GCTTAGTTGTACGCAAAACGAGAAAGCTTATGAGCACATGGTTTATAACTATCATA
AACTCAAAATTTTGGTTTTTTTGAAAGAAACTAATTATATGTAGAAAAGTTTTTTTAA
AAAAAATAAACATAGTTTAACAGTTTAGAAAACGTACTAACAGAAAATGAGAAAGTT
ACCGCTCAGATCTTAGACTTAACTCCATAGACTCATATGAAAGGTCACTCATAAAT
TATTTTTCACGAATAAGCTGTTTGGTATATAAATCAGCTCTCGGTCAACCGATGAG
GTTGTACTTTGGATACATGTGTGCGAGTGCATCTTTCGTGTATAACGTGGCCCTGT
TCTTCCCCTCCCCACACACATGAATGTGTGTGTATTTAAACGGCTTTTGGGGGGTC
ACCTTTCGCAGGTACTATACCCCAAGAGCTGAAAAAATTGCAAGGCCGGGGCTTA
GCCATATCTGCTAGCAGAAACCTGTAGGCTGGATCATGTACCAGCTGCATTTGAT
GCATACCCTATGCTTTAGCTAAGGAGGAGTACGATCGATTCATCAATATCGCTCGA
TCGGTGACGACGTCGTCCCTGCGACATCAGATCGTACTACTGCTACAGTACAGCT
TTTGCCTGTTCCAATCACTAACCAGCCTGCCTCTCTCTCTCTCTCTGCCATGGATG
GCTAGCTAGCTAGATCGATCTATCGATGCAGAGTGATTAGCTAGCTAGCTAGAATT
GGTGAATTGTGGTGACGACGAGATCGTTAGCAACAGTGGCCACGAGTCAAGATG
CTGTATATATGTATGGATGAGTCATCAGTGTGATGCATGATCTCACATCTCGTCAC
TGATGATCTCCAGCTTGAGCTTGCGCTGAGGTGAGCTGAGCTACACTGCTGCTTC
TTTGACCTTCTCCATCGATCGAATCCCGTGGTGATAAACTGTTAACTGAGGTAATT
GTAACTACTGCAGCTTCGTCTCTCTCTCTCTCTCTCTCACGAAAGATGCGTGATGC
TGATGCATATGCGGTTTTTTGGATGTACTGTTTGGACGGTTGCATACTGTGCCCGT
TCAATGTCAGCATCTCCATCTTCATATGTTGCTGCCCCGCCTCATCTCGATCGCCA
ACTTCTATTGTTTCGCTTGTGCTTATACTTATAAGTTAAAATTTAAATTTTAAAATTTA
GTTTTGAAGTTAATTTTAAGATTTTTTTTATCATAGTTTATTTCACACCATTATTTGCT
TTTCAGTAGTTTAATAACATATAAATAAAAGTTATATTTATAGGTTAGTTTTGAGAGC
ACTGCTCCCGTCCAGCAAACGGTACCCCCAGGTACCGGTACCCCTGGTACGAAA
CTTAATTTGACCATTGAATTAGAGCGGGGCACGATCGGGATTGAACTTAATCTCGC
GAGGACTCAGTCTCCTGCCCATGCGCTGCATCCGCATCTGAAGTCTACGCCGTGT
CACCGCCGCAATCCTTTCGCCCTACTCCGACGCGGTGTCCAAGTGCCGTCCTCTT
CCGGCCTCTGATGCGGTGGTGTGGCGGCTCGCCTCTCGGCTGCCCCTGTGCGG
TACGGCAGCTCGTCCTTCGTCCACGGTCACAACTTGTCCCCTCCCCTCCATCCTC
TATGCATCTCGGCTGGTGGCACCTACGCAGTCTCCGGCACAGGATCACAACACC
CTGAGTTTCTTTACGGAAGATGTATGAGAGAGATGAAGTATTTCTTTCCTGCTGGA
CATTGCTTTGCTGCTATATTCATTGGAAGAATCTGCTATGTTGATGGGAGAGGCTG
AGTTTGATTTATGTTGTGTGCCAAGTTCTGGACCTAGTTCCTTGTCTTTTGATATAT
GTAATGAACCTACTTGATTTTGCTGAAAGTATGAATGTAATAGTAGTAAAAAAGTAG
ATGTTCTGAAAGTTTGTAGTTTCTTGCTCTGATGTGTAAACTGTTCTTTCGTTGTAG
ACCTTAAGCTTACTGTTTCATCTTAAACAAAATTAACATCGGCGGTCATTGAGCAAA
TTGTCAACTATCTTGAATAAAAGGGACACTATTACAACTAGTGATCCCATAAATAAA
CTTCTGAAATTCTTCGATCTCTTTTCTTTGCTTGCCCAATTTCTTCTTGCTTGTGCG
ATCCATGGCCAAAAGCCTTTCAGCCATCTCAATATCTAGCTTCGTTTTTTCTTCTTT
CTGTCCCCTCCTTCTAGGTTTGCTAATGCTCGCGATCCAATGCATACCACAGCGAT
GCGATTCCCTGAACAAAGGCTTGCGATGGCGTCGGTAGACGAGCTGTCGAGGCC
ATATTCGCCGCGCTGAGCTAGTCGCCATGGTCACCTCTTCTTGTTGGTGCATTGA
GGTCATCATCATCCAGGGGTAGATCGACTCCCAGAAATCGGCGGCGGCCGGACC
TGGACCGGCTACGGGAAGGGCAAGTTGGGAATGGGCAGGTTGGCGACGGTTGG
TTGCAGTGAGGGAGGAAGCGAAGTCCGGCGAGATTGGCTGTGCCGCCGCCAGG
GACGACGGAAGGGGATCGGGAAGAGACAGATTACCGGGTCAGGGACTTGTACCG
TCTCTATGAAAATTGACTAACCACGTAAAAATCCAATTCTGCCCTTCCACTAGTTCC
ATACACGCGAACAAAGGGGGAGGTACCCTGCACCGGATTCCTGTACCTCGCGGT
ACCGATGATTGTGGGTCGTTTGATCTGGCTGAATGGACGGTTACGATTGCAGTAC
CGCGTGGTACCAAAAATTCTGCTGGACAGTAAAAAATCTCTTTGATTTGCTAATAA
GCAGTATGAATAAACATATGCATAAGCGAAAGGCTAGGTGCTACTACTTCTACTGT
GGAAGTGCTCATGGAGGGGGGCCGAATGGCCGATCGATCGAGAAAGCATTCATC
CATTCCATTCTCTCGCTCTCTCAAAAGTTGCAATCTTTTTTTTTTCTCCTCTTCTTCT
TTTTCTTTTTCGTTTTCCAGCTCATCTCTGATGGATCATCTCTCTCTCCCCGTGTGG
TAATTCCGATGTGATCGATGACGCGTGCATGCGTCGGAGTAGGAGTACAGCCTCT
GTTGTTCTTTTTGGTCTTCTTCTTCTACTTCCTCCCAAAAATGCGTTGTGAGCGAGA
AAAAGAGAAGCTTTTTTTTGGTGTGCGTGAGTGTGCAACTCTCAATATTTGTTGCC
CGAAATCTTTCGAGTTTGCGTCTTTTTGGGCTTACACTGTCCCTTTTTTATCGCTGC
GTCCAATCCTAATCCACTATTTATGCCTAATTAATTACTCCGTCTGTTCTTGAATAT
GACAACTTAACTTAAGTAGTGGATTGAACCTTACGTGGTACTGTACTATATATCTA
GACATACATTATATCTAGATATATTGTACCAGGTAATATCTCATATAGTACTAGGAT
GTTATATTCTCTGGTACTCTCTACCGGTAGTACAGTAGGAATGGTCTGAATTGTTC
GTTATTAAGGGTAAATTATAAATTTACTAATATAATGATTACGGAATACTTTATTATT
TATGTTTTTTTTGTCTAAATCAATTTATGGTCATCCATTTTATTGGCATCACTCATAT
TTTATCGAAAAGAAAAATAAAAGAGAAAGAAGATTTAGTCACTAATAAATGACGATT
ATACCTTACACCTATTTTTTTACAATAATGCTCCCTCAAAATTTCTATAACCACTAAC
TGAAATGGTAATACAAATAGATCTAAACCACTAGATAAAAAAGATAAACTGTTCATA
TTGCTTTCTATCAGTAGCCTGGGATTGGGACGGAGGAAGCGAAGAGAGAGAAAG
AGAGAGGTGGTTTTGTTTTGTTTGTCCGGTAATGGCTGCCGCATTGGTGGTGGTG
GCCTCCTCTCCTCTTCTTTTATTTCGAACGCGACGCCACCCACGCGCCTCCCCCT
CCCCCCTGCGGTTTCCCTCTCTTATTCAAAACCTGTCTCGATTCTCACTCACTCTC
ACTCACTCGGACTCCTCACCCGCTAGCTACCCCGGAGCGCGCCGCGCCACCGCT
CGACAGCGGCGAG
SEQ ID NO: 29 (DEL1)
TCATTTTTAAATGAAAGGCACCATCTTAGATTTTTTTTCCTTTAGTTGATTGGTTGTA
GTACTATATTTTCTAAGTTATTATAAAATAAATATGCGATATATATTTGATTGCCATT
ATCGAGTTAGTATATCTTCACTCTGTTTATATTTTAAAGCTGTGTACTCCTAGCTAT
AGCACTAAAGACCGAACTTAGTATGGACTAATTACAGCGATAACCATCGGTAATTG
CTACTACTCCATGCAGTACTTGGTTTCCATATTTTTGCATGCGTCGGTCGTTGGAG
GCAAGCAAGCAAGCTGCAGCGTCGTCAGAGGTAGACGCATCGATGTTGTTCTTCC
ATGTCCTTCGAATCTTCATGATTGCCGCTCTCTGCTTAATTACTGTTCTCTTCACGC
ACACCGTCCAAATCCCATCCATCTATCCATCCGTCCACACACCTGCAAGTTTAAAT
TCCCATGCAGTTACTACATGAACAGTACTGCTGTCAAGTTTAATTAATTTCTACTAC
ACACGAATTGAATTCAAATGGTACTAGGTACAAGTACTCCACTAGTACGACCATGA
TGTTTCCCACCTGAAAGCAACACAGAATACGTTCGTACGTACACGTACAGTAAACC
ACAATAATGCACAAGAGTAGGGATCGATGGAGAGTACATGCAATTGCTCATCACTT
GTCACCATCACTGGTTCAAATTTGCCTACTCTAGATCTCGATCGTGGTACTGCTTC
TAACCATAATGATCGGTACATGTTCAAATTCGAAACTCAAAAAATGTGTGTACAATA
CGGCCGGGAGAGATGAACATATATACAGTACTCATAATTATTAACCATGCATGGG
CATTAATAAAAGTAGTACTCCGTATATAGCTTCCAAGCAAGGCTACATACTAGAAT
CATACTGTATCTTGATTGCCAGAGGTGCAATCATGCATAGACATACTT
SEQ ID NO: 30 (DEL2)
TCTACTACACACGAATTGAATTCAAATGGTACTAGGTACAAGTACTCCACTAGTAC
GACCATGATGTTTCCCACCTGAAAGCAACACAGAATACGTTCGTACGTACACGTA
CAGTAAAACCACAATAATGCACAAGAGTAGGGATCGATGGAGAGTACATGCAATT
GCTCATCACTTGTCACCATCACTGGTTCAAATTTGCCTACTCTAGATCTCGATCGT
GGTACTGCTTCTAACCATAATGATCGGTACATGTTCAAATTCGAAACTCAAAAAAT
GTGTGTACAATACGGCCGGGAGAGATGAACATATATACAGTACTCATAATTATTAA
CCATGCATGGGCATTAATAAAAGTAGTACTCCGTATATAGCTTCCAAGCAAGGCTA
CATACTAGAATCATACTGTATCTTGATTGCCAGAGGTGCAATCATGCATAGACATA
CTTAAGATCAGGGCTAGCTAGTTCAGGTGATGAACACATGCTTTGTACCCCTTGTA
GGATGCATGGCACCAAGTCGACATTGGAGTACACACACCCTGGCCCTGCTGCAC
GTGTAGATAAGAGTACCACCTATCATCATCAATCACGCAATATGGTCTACTGTGTC
TCTTTGATTGGGTGTAATATAATATCGGCCACTGTAAAAATTTAAAATTTAAAACTT
AGTAATTTTAATTTCGAAGTTGATTTTATGATATGCTCAACGTATCTCTTTTTTTTCA
AGCTAGCGTTGGTCTTTAAAAGTGCGTTTGTTTATAAGGTTAGGGTGAGACTTTTA
GCTTGTGTTTGGTTAGTGGGATATGGAATGAGATGGGTTAGGTCGATCCTATTTTT
CGAGCTGTTTGGGTAGAGGGATGTGTAGGACGGGATGATCCTAGATGGGAATATT
CTTCCCAGATCCAGGACGAGGTGGTCCGCCAAAATCGGCTGGACTAATCCATCCC
ACTTTGATGGAGCGAATGGCATTAGCTCCTTCCGCCTCCTGCTCGGCTCCGCCAC
TGGCCGCCCGTTCCTCCTCCTCCGTCTGCTCCCTCCCACGGCGTCCGTCCCTCC
TCCTCTTCCTGCTCCCTCCCCCGATGCCGCTCCGTCCTCCTCCGCCTGCTCCCTC
CCCCGGCGCCCGTCCCTCCTCCTCTGCCCGCTCGCCAAGTTGCCGGCTGCACCG
CCCGCTCGTGCAGCTCCTCCTGCCATCCGCGCCGCCGGTCGCTGA
SEQ ID NO: 31 (IN1)
TTAAGGGCCCCTTTGAATCAAAGGATTTATGTAGGAATTTCATAGGATTCAAATCC
TATAGGAAATTTTCCTATTTGGCCCTTTAATTCAAAGGATTGAAGCTTTCCAAATCC
TATGAAATTCCTATGGAATGACACATTGCATGTAGATTTTGGAGGAAATTTAGCAA
GAGCTCCAACCTCTTGGAAAATTTCCTTTGAGTCTATCTCTCTCATCCGATTCCTG
CGTTTTTCCTGCACTCCAATCAAACGACCATTCCTGTGTTTTTCCTGTGTTTTGCAA
TCCTCTGTTTTACACTTCAATTCCTGTCAGAATCCTATGTTTTTCCTATTCCTCCGT
TTTTTCTACCCTGCGATTCAAAGGGGCC
SEQ ID NO: 32
Oryza sativa GSE5 genomic sequence
ATGGGCAAGGCGGCGCGGTGGTTCCGCAACATGTGGGGAGGAGGGAGGAAGGA
GCAGAAGGGCGAGGCGCCGGCGAGTGGGGGGAAGAGGTGGAGCTTCGGGAAG
TCGTCGAGGGACTCGGCGGAGGCCGCGGCGGCTGCTGCTGCGGCGGCGGCGG
AGGCTTCCGGGGGCAATGCGGCGATCGCCAGGGCGGCCGAGGCGGCGTGGCT
CAGGTCGGTGTACGCCGACACGGAGCGGGAGCAGAGCAAGCACGCCATCGCCG
TCGCCGCGGCCACCGCGGCGGCGGCTGATGCCGCCGTGGCGGCCGCTCAGGC
CGCCGTCGCCGTCGTGCGGCTTACTAGCAAGGGCCGCTCGGCTCCCGTCCTCGC
CGCCACCGTCGCCGGCGACACGCGCAGCCTTGCCGCCGCCGCCGTCAGAATCC
AGACGGCATTCAGAGGCTTCCTGgtaagcaacgggtgctcgtcttcttgctctggtttcg
tcgtcgtcgtcgtcgccattgtcgttaatcatggcgtgtgttcgtgcagGCGAAGAAGGC
GCTGCGAGCGCTCAAGGCGCTGGTGAAGCTGCAGGCGCTGGTGCGCGGCTACCTCGTTCG
CCGGCAGGCCGCCGCCACGCTGCAGAGCATGCAGGCGCTCGTCCGCGCCCAGGCCACTGT
CCGCGCCCACCGCAGTGGCGCCGGCGCCGCCGCCAATCTCCCGCACCTCCACCACGCT
CCCTTCTGGCCCCGCCGCTCGCTGgtacgccgctggctaaatctcgccgacgacatcgcc
atgtatatgttcgatgttgacgttgtgtgttggcgatggatgcagCAGGAGAGGTGCGCC
GGCGACGACACGAGGAGCGAGCACGGTGTGGCGGCGTACAGCCGGCGGCTGTCGGCGAGC
ATCGAGTCGTCGTCGTACGGGTACGACCGGAGCCCCAAGATCGTGGAGGTGGACACCGGG
AGGCCCAAGTCGCGGTCGTCGTCGTCGCGGCGGGCGAGCTCCCCGCTGCTGCTC
GACGCCGCTGGGTGCGCGAGCGGCGGCGAGGACTGGTGCGCCAACTCCATGTC
GTCGCCGCTCCCGTGCTACCTCCCCGGCGGCGCGCCGCCGCCCCGCATCGCCG
TCCCGACGTCGCGCCACTTCCCCGACTACGACTGGTGCGCGCTGGAGAAGGCCC
GGCCGGCGACGGCGCAGAGCACGCCGCGGTACGCGCACGCGCCGCCGACGCC
GACCAAGAGCGTGTGCGGCGGCGGCGGCGGCGGCGGCATCCACTCGTCGCCG
CTCAACTGCCCGAACTACATGTCCAACACGCAGTCGTTCGAGGCGAAGGTGCGTT
CGCAGAGCGCGCCGAAGCAGCGGCCGGAGACCGGCGGCGCCGGCGCCGGCGG
CGGCCGGAAGCGGGTGCCGCTGAGCGAGGTGGTGGTGGTGGAGTCCAGGGCG
AGCTTGAGCGGCGTGGGCATGCAGCGCTCGTGCAACCGGGTGCAGGAGGCGTT
CAACTTCAAGACGGCCGTCGTCGGCCGCCTCGACCGCTCGTCGGAGTCCGGCGA
GAACGACCGCCACGCGTTCTTGCAGAGGAGGTGGTGA
Uppercases: exon
Lowercases: intron
SEQ ID NO: 33
Cas9 sequence
GACAAGAAGTACAGCATCGGCCTGGACATCGGCACCAACTCTGTGGGCTGGGCC
GTGATCACCGACGAGTACAAGGTGCCCAGCAAGAAATTCAAGGTGCTGGGCAAC
ACCGACCGGCACAGCATCAAGAAGAACCTGATCGGAGCCCTGCTGTTCGACAGC
GGCGAAACAGCCGAGGCCACCCGGCTGAAGAGAACCGCCAGAAGAAGATACACC
AGACGGAAGAACCGGATCTGCTATCTGCAAGAGATCTTCAGCAACGAGATGGCCA
AGGTGGACGACAGCTTCTTCCACAGACTGGAAGAGTCCTTCCTGGTGGAAGAGG
ATAAGAAGCACGAGCGGCACCCCATCTTCGGCAACATCGTGGACGAGGTGGCCT
ACCACGAGAAGTACCCCACCATCTACCACCTGAGAAAGAAACTGGTGGACAGCAC
CGACAAGGCCGACCTGCGGCTGATCTATCTGGCCCTGGCCCACATGATCAAGTT
CCGGGGCCACTTCCTGATCGAGGGCGACCTGAACCCCGACAACAGCGACGTGGA
CAAGCTGTTCATCCAGCTGGTGCAGACCTACAACCAGCTGTTCGAGGAAAACCCC
ATCAACGCCAGCGGCGTGGACGCCAAGGCCATCCTGTCTGCCAGACTGAGCAAG
AGCAGACGGCTGGAAAATCTGATCGCCCAGCTGCCCGGCGAGAAGAAGAATGGC
CTGTTCGGAAACCTGATTGCCCTGAGCCTGGGCCTGACCCCCAACTTCAAGAGCA
ACTTCGACCTGGCCGAGGATGCCAAACTGCAGCTGAGCAAGGACACCTACGACG
ACGACCTGGACAACCTGCTGGCCCAGATCGGCGACCAGTACGCCGACCTGTTTC
TGGCCGCCAAGAACCTGTCCGACGCCATCCTGCTGAGCGACATCCTGAGAGTGA
ACACCGAGATCACCAAGGCCCCCCTGAGCGCCTCTATGATCAAGAGATACGACG
AGCACCACCAGGACCTGACCCTGCTGAAAGCTCTCGTGCGGCAGCAGCTGCCTG
AGAAGTACAAAGAGATTTTCTTCGACCAGAGCAAGAACGGCTACGCCGGCTACAT
TGACGGCGGAGCCAGCCAGGAAGAGTTCTACAAGTTCATCAAGCCCATCCTGGA
AAAGATGGACGGCACCGAGGAACTGCTCGTGAAGCTGAACAGAGAGGACCTGCT
GCGGAAGCAGCGGACCTTCGACAACGGCAGCATCCCCCACCAGATCCACCTGGG
AGAGCTGCACGCCATTCTGCGGCGGCAGGAAGATTTTTACCCATTCCTGAAGGAC
AACCGGGAAAAGATCGAGAAGATCCTGACCTTCCGCATCCCCTACTACGTGGGCC
CTCTGGCCAGGGGAAACAGCAGATTCGCCTGGATGACCAGAAAGAGCGAGGAAA
CCATCACCCCCTGGAACTTCGAGGAAGTGGTGGACAAGGGCGCTTCCGCCCAGA
GCTTCATCGAGCGGATGACCAACTTCGATAAGAACCTGCCCAACGAGAAGGTGCT
GCCCAAGCACAGCCTGCTGTACGAGTACTTCACCGTGTATAACGAGCTGACCAAA
GTGAAATACGTGACCGAGGGAATGAGAAAGCCCGCCTTCCTGAGCGGCGAGCAG
AAAAAGGCCATCGTGGACCTGCTGTTCAAGACCAACCGGAAAGTGACCGTGAAG
CAGCTGAAAGAGGACTACTTCAAGAAAATCGAGTGCTTCGACTCCGTGGAAATCT
CCGGCGTGGAAGATCGGTTCAACGCCTCCCTGGGCACATACCACGATCTGCTGA
AAATTATCAAGGACAAGGACTTCCTGGACAATGAGGAAAACGAGGACATTCTGGA
AGATATCGTGCTGACCCTGACACTGTTTGAGGACAGAGAGATGATCGAGGAACGG
CTGAAAACCTATGCCCACCTGTTCGACGACAAAGTGATGAAGCAGCTGAAGCGGC
GGAGATACACCGGCTGGGGCAGGCTGAGCCGGAAGCTGATCAACGGCATCCGG
GACAAGCAGTCCGGCAAGACAATCCTGGATTTCCTGAAGTCCGACGGCTTCGCCA
ACAGAAACTTCATGCAGCTGATCCACGACGACAGCCTGACCTTTAAAGAGGACAT
CCAGAAAGCCCAGGTGTCCGGCCAGGGCGATAGCCTGCACGAGCACATTGCCAA
TCTGGCCGGCAGCCCCGCCATTAAGAAGGGCATCCTGCAGACAGTGAAGGTGGT
GGACGAGCTCGTGAAAGTGATGGGCCGGCACAAGCCCGAGAACATCGTGATCGA
AATGGCCAGAGAGAACCAGACCACCCAGAAGGGACAGAAGAACAGCCGCGAGAG
AATGAAGCGGATCGAAGAGGGCATCAAAGAGCTGGGCAGCCAGATCCTGAAAGA
ACACCCCGTGGAAAACACCCAGCTGCAGAACGAGAAGCTGTACCTGTACTACCTG
CAGAATGGGCGGGATATGTACGTGGACCAGGAACTGGACATCAACCGGCTGTCC
GACTACGATGTGGACCATATCGTGCCTCAGAGCTTTCTGAAGGACGACTCCATCG
ACAACAAGGTGCTGACCAGAAGCGACAAGAACCGGGGCAAGAGCGACAACGTGC
CCTCCGAAGAGGTCGTGAAGAAGATGAAGAACTACTGGCGGCAGCTGCTGAACG
CCAAGCTGATTACCCAGAGAAAGTTCGACAATCTGACCAAGGCCGAGAGAGGCG
GCCTGAGCGAACTGGATAAGGCCGGCTTCATCAAGAGACAGCTGGTGGAAACCC
GGCAGATCACAAAGCACGTGGCACAGATCCTGGACTCCCGGATGAACACTAAGTA
CGACGAGAATGACAAGCTGATCCGGGAAGTGAAAGTGATCACCCTGAAGTCCAA
GCTGGTGTCCGATTTCCGGAAGGATTTCCAGTTTTACAAAGTGCGCGAGATCAAC
AACTACCACCACGCCCACGACGCCTACCTGAACGCCGTCGTGGGAACCGCCCTG
ATCAAAAAGTACCCTAAGCTGGAAAGCGAGTTCGTGTACGGCGACTACAAGGTGT
ACGACGTGCGGAAGATGATCGCCAAGAGCGAGCAGGAAATCGGCAAGGCTACCG
CCAAGTACTTCTTCTACAGCAACATCATGAACTTTTTCAAGACCGAGATTACCCTG
GCCAACGGCGAGATCCGGAAGCGGCCTCTGATCGAGACAAACGGCGAAACCGG
GGAGATCGTGTGGGATAAGGGCCGGGATTTTGCCACCGTGCGGAAAGTGCTGAG
CATGCCCCAAGTGAATATCGTGAAAAAGACCGAGGTGCAGACAGGCGGCTTCAG
CAAAGAGTCTATCCTGCCCAAGAGGAACAGCGATAAGCTGATCGCCAGAAAGAAG
GACTGGGACCCTAAGAAGTACGGCGGCTTCGACAGCCCCACCGTGGCCTATTCT
GTGCTGGTGGTGGCCAAAGTGGAAAAGGGCAAGTCCAAGAAACTGAAGAGTGTG
AAAGAGCTGCTGGGGATCACCATCATGGAAAGAAGCAGCTTCGAGAAGAATCCCA
TCGACTTTCTGGAAGCCAAGGGCTACAAAGAAGTGAAAAAGGACCTGATCATCAA
GCTGCCTAAGTACTCCCTGTTCGAGCTGGAAAACGGCCGGAAGAGAATGCTGGC
CTCTGCCGGCGAACTGCAGAAGGGAAACGAACTGGCCCTGCCCTCCAAATATGT
GAACTTCCTGTACCTGGCCAGCCACTATGAGAAGCTGAAGGGCTCCCCCGAGGA
TAATGAGCAGAAACAGCTGTTTGTGGAACAGCACAAGCACTACCTGGACGAGATC
ATCGAGCAGATCAGCGAGTTCTCCAAGAGAGTGATCCTGGCCGACGCTAATCTGG
ACAAAGTGCTGTCCGCCTACAACAAGCACCGGGATAAGCCCATCAGAGAGCAGG
CCGAGAATATCATCCACCTGTTTACCCTGACCAATCTGGGAGCCCCTGCCGCCTT
CAAGTACTTTGACACCACCATCGACCGGAAGAGGTACACCAGCACCAAAGAGGTG
CTGGACGCCACCCTGATCCACCAGAGCATCACCGGCCTGTACGAGACACGGATC
GACCTGTCTCAGCTGGGAGGCGAC
Primers for CRISPR/Cas9
Arabidopsis thaliana:
(SEQ ID NO: 34)
ggcaACAGAACAAACACGCGATTG
(SEQ ID NO: 35)
aaacCAATCGCGTGTTTGTTCTGT
Glycine max:
(SEQ ID NO: 36)
ggcaAGGCTGCGGTGGCGGTTGTT
(SEQ ID NO: 37)
aaacAACAACCGCCACCGCAGCCT
Medicago truncatula:
(SEQ ID NO: 38)
ggcaTTCTCAAAGAGAGATTCGAG
(SEQ ID NO: 39)
aaacCTCGAATCTCTCTTTGAGAA
Triticum aestivum:
(SEQ ID NO: 40)
ggcaCAGCAAAGGGCCGACGTCGA
(SEQ ID NO: 41)
aaacTCGACGTCGGCCCTTTGCTG
Zea mays:
(SEQ ID NO: 42)
ggcaCCGCGTGCGCCGAGACGCAC
(SEQ ID NO: 43)
aaacGTGCGTCTCGGCGCACGCGG
Oryza sativa:
(SEQ ID NO: 44)
ggcaCGAGGCGGCGTGGCTCAGGT
(SEQ ID NO: 45)
aaacACCTGAGCCACGCCGCCTCG
Sorghum bicolor
(SEQ ID NO: 46)
ggcaGTCGAGTCCTCGTCGTACGG
(SEQ ID NO: 47)
aaacCCGTACGACGAGGACTCGAC
SEQ ID NO: 48: Sorghum bicolor target sequence
GTCGAGTCCTCGTCGTACGGCGG
Setaria italica
(SEQ ID NO: 49)
CDS:
ATGGGCAAGGCGGCGCGGTGGTTCCGCAGCTTCCTGGGCAAGAAGGAGCAGGC
CAGTAAAGACCAGAGGCGGCAGCAGGACCAGCCGCCGCCCCCGCCGGCCACCG
CCAAGCGCTGGAGCTTCGGCAAGTCCTCCCGGGACTCGGCGGAGGCCGCCGCG
GCCGCGGCCGCGGGCGCCGTGTCGGCCGGCTCGGGCAACGCGGCGATCGCGC
GCGCGGCGGAGGCCGCGTGGCTCAGGTCCGCGGCCTACGACGAGACGAACAGG
GAGCGGGAGCAGAGCAAGCACGCCATCGCCGTGGCCGCGGCCACTGCGGCGG
CGGCGGACGCGGCGGTGGCCGCGGCCCAGGCGGCCGTCGCCGTCGTGCGGCT
CACCAGCAAGGGCCGCGCCGCGCCCACCCTCGCCACCGCCGCCGGCGGCCGC
GCCGCTGCCGCCGTCAGGATCCAGACGGCGTTCCGAGGATTCTTGGCGAAGAAG
GCGTTGCGCGCGCTCAAGGCGCTTGTGAAGCTGCAGGCGCTGGTGCGGGGCTA
CCTCGTGCGCAGGCAGGCGGCCGCCACGCTCCACAGCATGCAGGCCCTCGTCC
GCGCTCAGGCCACCGTGCGCGCGCACCGCGCCGGCGTCCCAGTCGTCTTCCCG
CACCTCCACCACCCGCCCGTCCGGCCGCGCTACTCGCTGCAAGAGCGGTACGCC
GACGACACGCGGAGCGAGCACGGCGCGCCGGCGTACGGCAGCCGGCGGATGT
CGGCGAGCGTCGAGTCCTCGTCGTACGCGTACGACCGGAGCCCCAAGATCGTG
GAGGTGGACCCGGGGCGGCCCAAGTCGCGCTCATCCTCGCGTCGCGCGAGCTC
CCCGCTGGTCGACGCCGGCAGCAGCGGTGGCGAGGAGTGGTGCGCTAACTCCG
CGTGCTCGCCGCTGCCGTGCTACCTGTCCGGCGGCCCGCCGCAGCCGCCGCGC
ATCGCCGTGCCAACCTCGCGCCAGTTCCCGGACTACGACTGGTGCGCGCTGGAG
AAGGCGCGGCCGGCGACGGCGCAGAACACGCCGCGGTACCTGCACGTGCACGC
GCACGCGCCGGCCACCCCGACCAAGTCCGTGGCGGGCTACTCGCCGTCGCTCA
ACGGCTGCCGGAACTACATGTCGAGCACGCAGGCTTCGGAGGCGAAGGTGCGG
TCGCAGAGCGCGCCGAAGCAGCGGCCGGAGCTCGCCTGCGGCGGCGGCGCTC
GGAAGCGGGTGCCGCTGAGCGAGGTGGTGGTGGTGGAGTCGTCCCGCGCGAGC
CTGAGCGGCGTCGTCGGCATGCAGCGCGGGTGCGGCGGCCGCGCGCACGAGG
CGTTCAGCTTCAAGTCCGCCGTCGTCGGCCGCATCGACCGCACGCTGGAGGTGG
CCGGCGTCGAGAACGACCGCCTGGCGTTCCTGCAGAGGAGGTGGTGA
(SEQ ID NO: 50)
PROTEIN:
MGKAARWFRSFLGKKEQASKDQRRQQDQPPPPPATAKRWSFGKSSRDSAEAAAAA
AAGAVSAGSGNAAIARAAEAAWLRSAAYDETNREREQSKHAIAVAAATAAAADAAVA
AAQAAVAVVRLTSKGRAAPTLATAAGGRAAAAVRIQTAFRGFLAKKALRALKALVKLQ
ALVRGYLVRRQAAATLHSMQALVRAQATVRAHRAGVPVVFPHLHHPPVRPRYSLQE
RYADDTRSEHGAPAYGSRRMSASVESSSYAYDRSPKIVEVDPGRPKSRSSSRRASS
PLVDAGSSGGEEWCANSACSPLPCYLSGGPPQPPRIAVPTSRQFPDYDWCALEKAR
PATAQNTPRYLHVHAHAPATPTKSVAGYSPSLNGCRNYMSSTQASEAKVRSQSAPK
QRPELACGGGARKRVPLSEVVVVESSRASLSGVVGMQRGCGGRAHEAFSFKSAVV
GRIDRTLEVAGVENDRLAFLQRRW
(SEQ ID NO: 51)
target sequence
CGTGGCCGCGGCCACTGCGGCGG
(SEQ ID NO: 52)
protospacer sequence
CGTGGCCGCGGCCACTGCGG
Setaria italica Primers for CRISPR/Cas9:
(SEQ ID NO: 53)
ggca CGTGGCCGCGGCCACTGCGG
(SEQ ID NO: 54)
aaac CCGCAGTGGCCGCGGCCACG
vectors: SK-gRNA & pC1300-Cas9
GSE5-Like
SEQ ID NO: 55; genomic Sequence; LOC_Os01g09470
GCGTTTCCCTCTCTTATTCAAACTTGACCCGTTTCGCCTTCTTGCTCAAGTGTTCG
ACCTGGTCTTGGAGCGCGGCGTGTCTCTCTCGCCGGCCGGAGTCGCGAATTCCG
GCCATGGGCAAGGCGGCGAGGTGGTTCCGCAGCCTGTGGGGCGGCGGCGGCG
GGAAGAAGGAGCAGGGGAGAGAACATGGGAGGACGGCCGCGGCGCCGCCCCC
GCCGGACAGGAAGCGGTGGAGCTTCGCCAAGTCGTCGAGGGACTCGACGGAGG
GGGAGGCGGCGGCGGCGGTGGGAGGGAATGCGGCGATCGCGAAGGCGGCCGA
GGCGGCGTGGCTCAAGTCGATGTACAGCGACACCGAGAGGGAGCAGAGCAAGC
ACGCCATCGCGGTCGCCGCGGCGACCGCGGCTGCGGCGGACGCGGCCGTGGC
GGCGGCACAGGCGGCCGTCGAGGTCGTCCGCCTCACCAGCCAGGGGCCACCCA
CCTCGTCGGTGTTCGTCTGCGGCGGCGTCTTGGATCCCCGTGGCCGCGCCGCC
GCGGTCAAGATCCAGACAGCCTTCCGAGGATTCTTGGTGAGTGAGCCCCAACAA
CTTCCTCACTTCTTCCAAGAACAACAGTGTCTGCTTCTGTTCTTGATCTGTTCGTCT
TCTTTGGCGACGTGCTCATTTCGATTTCATCCACTGTTCCAGTAGATTTCCTTTTCC
AAAAAAAGCTCATAGATTAAGACATGATTAGATTTTTATTTTTGTTCTTGGTTCAGG
CGAAGAAGGCGCTGCGAGCGCTCAAGGCGCTGGTGAAGCTGCAGGCGCTGGTG
CGCGGCTACCTGGTGAGGCGGCAGGCGGCGGCGACGCTGCAGAGCATGCAGGC
GCTCGTCCGCGCGCAGGCCGCCGTCCGCGCCGCGCGCTCGTCGCGCGGCGCC
GCGCTGCCGCCGCTGCACCTCCACCACCACCCTCCCGTCCGGCCGCGCTATTCC
CTGGTACGAGTACGACCACGATCGCTTGCGTGCGAAGCGGGCGAGCTTTTTTTTT
AAAGGTGTTCGTCCGAGGCATGTTGGTTGCTGTGACACAATTCTTACCTCGGGGG
TTTCTTGTGTTTGCAGCAAGAGCGGTATATGGACGACACGAGGAGCGAGCATGGC
GTGGCGGCGTACAGCCGCCGCCTGTCGGCGAGCATCGAGTCGTCGTCGTACGG
GTACGACCGGAGCCCCAAGATCGTGGAGATGGACACCGGGCGGCCCAAGTCGA
GGTCGTCGTCGGTCAGGACGAGCCCTCCCGTGGTCGACGCCGGCGCCGCCGAG
GAGTGGTACGCCAACTCGGTGTCGTCGCCGCTCCTCCCGTTCCACCAGCTCCCC
GGCGCGCCGCCGCGGATATCGGCGCCGAGCGCACGCCACTTCCCGGAGTACGA
CTGGTGCCCGCTCGAGAAGCCCAGGCCGGCGACGGCGCAGAGCACGCCGCGG
CTTGCGCACATGCCGGTGACGCCGACGAAGAGCGTCTGCGGCGGCGGCGGCTA
CGGCGCGTCGCCCAACTGCCGCGGCTACATGTCGAGCACGCAATCGTCGGAGG
CGAAGGTGCGGTCCCAGAGCGCGCCGAAGCAGCGGCCGGAGCCGGGCGTCGC
CGGCGGCACCGGCGGCGGCGCGCGGAAGAGGGTGCCGCTGAGCGAGGTGACC
CTGGAGGCGAGGGCGAGCCTGAGCGGCGTGGGCATGCAGCGCTCGTGCAACCG
TGTCCAGGAGGCGTTCAACTTCAAGACCGCCGTGCTCAGCCGCTTCGACCGCTC
GTCGGAGCCGGCCGCCGAGAGGGACCGCGACCTCTTCTTGCAGAGGAGGTGGT
GATCTGAACAGCGTTCGCCATTGCAAGAAGGAAGAGGACTACAAGAACTAGTTCT
TCTTCTTCTTCTTAGTCTCTGTTTCTATGCGACATAGTAGCGATCGATCATGTTTGA
TCGATGGCAATGGCGATCGTGTGCTCCGCCATTGCCGTCGTCTCCGAGCTTGTTA
CTGACAAGTGACAGGCAAAGTGTACGTTGAGCTAGCTGGAGGGGAGATTACAAAA
AAAAAAAATCCCACTTCTTTCCCCTCTGATTTAACAGTGCACTTGGATGTACATTCC
CCTATCAATTCAAGGCCAGCAAATCAAATCCCGTTGTTTTTTTTTAA
SEQ ID NO: 56: >LOC_Os01g09470.1
ATGGGCAAGGCGGCGAGGTGGTTCCGCAGCCTGTGGGGCGGCGGCGGCGGGA
AGAAGGAGCAGGGGAGAGAACATGGGAGGACGGCCGCGGCGCCGCCCCCGCC
GGACAGGAAGCGGTGGAGCTTCGCCAAGTCGTCGAGGGACTCGACGGAGGGGG
AGGCGGCGGCGGCGGTGGGAGGGAATGCGGCGATCGCGAAGGCGGCCGAGGC
GGCGTGGCTCAAGTCGATGTACAGCGACACCGAGAGGGAGCAGAGCAAGCACG
CCATCGCGGTCGCCGCGGCGACCGCGGCTGCGGCGGACGCGGCCGTGGCGGC
GGCACAGGCGGCCGTCGAGGTCGTCCGCCTCACCAGCCAGGGGCCACCCACCT
CGTCGGTGTTCGTCTGCGGCGGCGTCTTGGATCCCCGTGGCCGCGCCGCCGCG
GTCAAGATCCAGACAGCCTTCCGAGGATTCTTGGCGAAGAAGGCGCTGCGAGCG
CTCAAGGCGCTGGTGAAGCTGCAGGCGCTGGTGCGCGGCTACCTGGTGAGGCG
GCAGGCGGCGGCGACGCTGCAGAGCATGCAGGCGCTCGTCCGCGCGCAGGCC
GCCGTCCGCGCCGCGCGCTCGTCGCGCGGCGCCGCGCTGCCGCCGCTGCACCT
CCACCACCACCCTCCCGTCCGGCCGCGCTATTCCCTGCAAGAGCGGTATATGGA
CGACACGAGGAGCGAGCATGGCGTGGCGGCGTACAGCCGCCGCCTGTCGGCGA
GCATCGAGTCGTCGTCGTACGGGTACGACCGGAGCCCCAAGATCGTGGAGATGG
ACACCGGGCGGCCCAAGTCGAGGTCGTCGTCGGTCAGGACGAGCCCTCCCGTG
GTCGACGCCGGCGCCGCCGAGGAGTGGTACGCCAACTCGGTGTCGTCGCCGCT
CCTCCCGTTCCACCAGCTCCCCGGCGCGCCGCCGCGGATATCGGCGCCGAGCG
CACGCCACTTCCCGGAGTACGACTGGTGCCCGCTCGAGAAGCCCAGGCCGGCG
ACGGCGCAGAGCACGCCGCGGCTTGCGCACATGCCGGTGACGCCGACGAAGAG
CGTCTGCGGCGGCGGCGGCTACGGCGCGTCGCCCAACTGCCGCGGCTACATGT
CGAGCACGCAATCGTCGGAGGCGAAGGTGCGGTCCCAGAGCGCGCCGAAGCAG
CGGCCGGAGCCGGGCGTCGCCGGCGGCACCGGCGGCGGCGCGCGGAAGAGG
GTGCCGCTGAGCGAGGTGACCCTGGAGGCGAGGGCGAGCCTGAGCGGCGTGG
GCATGCAGCGCTCGTGCAACCGTGTCCAGGAGGCGTTCAACTTCAAGACCGCCG
TGCTCAGCCGCTTCGACCGCTCGTCGGAGCCGGCCGCCGAGAGGGACCGCGAC
CTCTTCTTGCAGAGGAGGTGGTGA
SEQ ID NO: 57; Protein; >LOC_Os01g09470.1
MGKAARWFRSLWGGGGGKKEQGREHGRTAAAPPPPDRKRWSFAKSSRDSTEGEA
AAAVGGNAAIAKAAEAAWLKSMYSDTEREQSKHAIAVAAATAAAADAAVAAAQAAVE
VVRLTSQGPPTSSVFVCGGVLDPRGRAAAVKIQTAFRGFLAKKALRALKALVKLQALV
RGYLVRRQAAATLQSMQALVRAQAAVRAARSSRGAALPPLHLHHHPPVRPRYSLQE
RYMDDTRSEHGVAAYSRRLSASIESSSYGYDRSPKIVEMDTGRPKSRSSSVRTSPPV
VDAGAAEEWYANSVSSPLLPFHQLPGAPPRISAPSARHFPEYDWCPLEKPRPATAQS
TPRLAHMPVTPTKSVCGGGGYGASPNCRGYMSSTQSSEAKVRSQSAPKQRPEPGV
AGGTGGGARKRVPLSEVTLEARASLSGVGMQRSCNRVQEAFNFKTAVLSRFDRSSE
PAAERDRDLFLQRRW*
Z.mays
SEQ ID NO: 58; XM_008675371; Genomic Sequence:
AGCCACGACGTCACTGCCGCCATTAGACACATCACCGCCAGCAGCAGCGCCAGC
ACCTTCGTCGCGCCTTTTGAACTGATCCTGCCGCTTTTTGAACTCATCGTCCACGG
CGCACGCACCAACTCAAAAAAACCTCTTGGATTTGGGACGGCGACGCCCATTTCT
TTTTTCTTTAATCCCGTCGTCCATCTCGTGCCTTTGCGCAGCCAGCTACTAGGGCG
TCAGCTAAGTCGGTTTACACGCCGCACAAAAAACACACGAATATTCAGCTAGTAG
CAGCGTGAGGAGAGGAGAGGGTCCCCCGACCCGTCGTCCATCTCGTGCCTTTGC
GCAGCCAGCTACTAGGGCGTCAGCTAAGTCGGTTTACACGCCGCACAAAAAACAC
ACGAATATTCAGCTAGTAGCAGCGTGAGGAGAGGAGAGGGTCCCCCGACCCGTC
GTCTCTGCCTTCTTGCGCTGCATCTTTCCGGGTGCTCTTGTCCGCTAATGGCCCG
CCCTCCTCTCGTTTTATTCCAAACCCGCTCCTCCCCTGCTTTCCCTCTCTTATTCAA
ACTCGCAGTCCCAGTCCCAGGCTCCATTTTTCTAACTCCACCGGCCGTTGCCACC
CCCTCACTTCAGCTGCTTCTAGTTCTACCGCACCTCAGTGACTCAGTCCCCCGCT
AGGCTCGGAGCGGAGCGGAGCCGAGCTTCACTTGCCGGCTGCGAATTCCGGGG
ATGGGCAAGGCGGCGAGGTGGCTCCGCGGCCTGCTCGGCGGCGGGAGGAAAG
ACCAGGAGAGGCGGGCCTCGCCGGCGCCGCCCACCGCGGACAGGAAGCGCTG
GAGCTTCGCGAGGTCCTCGCGGGACTCGGCCGAGGCCGCCGCGGCGGCGACC
GAGGGCTCCGTGCGCGGCGGTGGCAACGCGGCGATCGCGCGGGCCGCCGAGG
CCGCGTGGCTCAAGTCGCTCTACGACGACACGGGGCGGCAGCAGAGCAAGCAC
GCCATCGCCGTCGCCGCGGCCACAGCGGCGGCGGCGGACGCGGCCGTGGCCG
CCGCGCAGGCCGCGGTCGAGGTCGTCCGGCTCACCAGCCAGGGCCCGGTCTTC
GGCGGCGGAGGGCCGGTGCCCGTGCTGGACCCCCGCGGCCGCGCCGGCGCCG
CCGTCAAGATCCAGACGGCATTCAGACGCTTCTTGGCGAAGAAGGCGCTGCGAG
CGCTGAAGGCGCTGGTGAAGCTGCAGGCGCTGGTGCGCGGCTACCTGGTGCGG
CGGCAGGCGGCGGCGACGCTGCAGAGCATGCAGGCGCTCGTCCGCGCGCAGG
CAGCCGTCCGCGCCGCCCGCTACAGCCGCGCGCTACCCGCGCTCCCGCCCCTC
CACCACCACCCTCCCGTCCGCGCGCGCTTCTCGCTGCAAGAGCGGTACGGTGAC
GACACGCGCAGCGAGCACGGCGTGGCGGCGTACAGCCGGCGCTTGTCCGCGAG
CATCGAGTCGGCGTCGTACGGAGGCGGGTACGACCGGAGCCCCAAGATCGTGG
AGATGGACACGGCGCGGCCCCGGTCGCGCGCGTCGTCCCTGCGCACCGAGGAC
GAGTGGTACGCGCAGTCGGTGTCGTCGCCGCTCCTGCCGCCGCCGCCGCCGCC
GCCGTGCCAGCACCTGCACCAGTACCACCACCTGCCCCCGCGCATCGCGGTGCC
CACGTCGCGCCACTTCCCGGACTACGACTGGTGCGCGCCGGAGAAGCCGCGGC
CGGCGACGGCGCAGTGCACGCCCCGCTGCGCGCCGCCGACCCCCGCCAGGAG
CGTCTGCGGCGCCGGGGGCAACGGCGGCGGCTACCTCGCCGCGTCGCCCGGC
TGTCCCGGGTACATGTCGAGCACGCGGTCGTCGGAGGCCAAGTCGTCGTCCCGG
TCGCAGAGCGCGCCGAAGCAGCGGCCGCTGGAGCAGCAGGAGCAGCAGCAGCA
GCCGGCCCGGAAGCGGGTGCCGCTCAGCGAGGTGGTCCTGGAGGCCCGCGCG
AGCCTGGGCGGCGCCGGCGTGGGCATGCACAAGCCGTGCAATACCCGCGCGCA
GGCGCAGGACGCGTTCGACTTCCGCACCGCCGTCGTGAGCCGGTTCGATCGGC
GCGCGTCGGACGCCGCCGCGGCGGCGGCCGAGCGGCGGGATCGCGAATTGTT
CTTCCTGCAGAGGAGGTGGTGAAGGTGAACCGATCGACCGCCCGACCGGGATGA
TTAATCGGGTGCTGCTAATAGGGAAGGCTCTCATTAATTCCTTTTCAGCATGCATC
TCCTCGCTGATCCCTGTTGTTCCGATCCCATCCGTGACCTCTCACTGCTGTCGTTC
TTCCTGCTTGCAATAAGCTAGTGTGTGTCGGGGAAGTAGGGAGAGATTTCATCCC
GTCCCGTACCGTTTGATTTCGTTTTTGCGTTCATAAACAGTAGCGCGGCTGGATC
GTCATCATCTCGATCCATGCATGTACATTCCGCCTGTTCCCCAATCACCATCAATC
AACAAGAAAGAGAAGCACGGTGTCGTTTCCGAGCCA
SEQ ID NO: 59; CDS:
ATGGGCAAGGCGGCGAGGTGGCTCCGCGGCCTGCTCGGCGGCGGGAGGAAAG
ACCAGGAGAGGCGGGCCTCGCCGGCGCCGCCCACCGCGGACAGGAAGCGCTG
GAGCTTCGCGAGGTCCTCGCGGGACTCGGCCGAGGCCGCCGCGGCGGCGACC
GAGGGCTCCGTGCGCGGCGGTGGCAACGCGGCGATCGCGCGGGCCGCCGAGG
CCGCGTGGCTCAAGTCGCTCTACGACGACACGGGGCGGCAGCAGAGCAAGCAC
GCCATCGCCGTCGCCGCGGCCACAGCGGCGGCGGCGGACGCGGCCGTGGCCG
CCGCGCAGGCCGCGGTCGAGGTCGTCCGGCTCACCAGCCAGGGCCCGGTCTTC
GGCGGCGGAGGGCCGGTGCCCGTGCTGGACCCCCGCGGCCGCGCCGGCGCCG
CCGTCAAGATCCAGACGGCATTCAGACGCTTCTTGGCGAAGAAGGCGCTGCGAG
CGCTGAAGGCGCTGGTGAAGCTGCAGGCGCTGGTGCGCGGCTACCTGGTGCGG
CGGCAGGCGGCGGCGACGCTGCAGAGCATGCAGGCGCTCGTCCGCGCGCAGG
CAGCCGTCCGCGCCGCCCGCTACAGCCGCGCGCTACCCGCGCTCCCGCCCCTC
CACCACCACCCTCCCGTCCGCGCGCGCTTCTCGCTGCAAGAGCGGTACGGTGAC
GACACGCGCAGCGAGCACGGCGTGGCGGCGTACAGCCGGCGCTTGTCCGCGAG
CATCGAGTCGGCGTCGTACGGAGGCGGGTACGACCGGAGCCCCAAGATCGTGG
AGATGGACACGGCGCGGCCCCGGTCGCGCGCGTCGTCCCTGCGCACCGAGGAC
GAGTGGTACGCGCAGTCGGTGTCGTCGCCGCTCCTGCCGCCGCCGCCGCCGCC
GCCGTGCCAGCACCTGCACCAGTACCACCACCTGCCCCCGCGCATCGCGGTGCC
CACGTCGCGCCACTTCCCGGACTACGACTGGTGCGCGCCGGAGAAGCCGCGGC
CGGCGACGGCGCAGTGCACGCCCCGCTGCGCGCCGCCGACCCCCGCCAGGAG
CGTCTGCGGCGCCGGGGGCAACGGCGGCGGCTACCTCGCCGCGTCGCCCGGC
TGTCCCGGGTACATGTCGAGCACGCGGTCGTCGGAGGCCAAGTCGTCGTCCCGG
TCGCAGAGCGCGCCGAAGCAGCGGCCGCTGGAGCAGCAGGAGCAGCAGCAGCA
GCCGGCCCGGAAGCGGGTGCCGCTCAGCGAGGTGGTCCTGGAGGCCCGCGCG
AGCCTGGGCGGCGCCGGCGTGGGCATGCACAAGCCGTGCAATACCCGCGCGCA
GGCGCAGGACGCGTTCGACTTCCGCACCGCCGTCGTGAGCCGGTTCGATCGGC
GCGCGTCGGACGCCGCCGCGGCGGCGGCCGAGCGGCGGGATCGCGAATTGTT
CTTCCTGCAGAGGAGGTGGTGA
SEQ ID NO: 60; Protein:
MGKAARWLRGLLGGGRKDQERRASPAPPTADRKRWSFARSSRDSAEAAAAATEGS
VRGGGNAAIARAAEAAWLKSLYDDTGRQQSKHAIAVAAATAAAADAAVAAAQAAVEV
VRLTSQGPVFGGGGPVPVLDPRGRAGAAVKIQTAFRRFLAKKALRALKALVKLQALV
RGYLVRRQAAATLQSMQALVRAQAAVRAARYSRALPALPPLHHHPPVRARFSLQER
YGDDTRSEHGVAAYSRRLSASIESASYGGGYDRSPKIVEMDTARPRSRASSLRTEDE
WYAQSVSSPLLPPPPPPPCQHLHQYHHLPPRIAVPTSRHFPDYDWCAPEKPRPATA
QCTPRCAPPTPARSVCGAGGNGGGYLAASPGCPGYMSSTRSSEAKSSSRSQSAPK
QRPLEQQEQQQQPARKRVPLSEVVLEARASLGGAGVGMHKPCNTRAQAQDAFDFR
TAVVSRFDRRASDAAAAAAERRDRELFFLQRRW
Sorghum bicolor:
SEQ ID NO: 61; XM_002457155; Genomic Sequence:
CATTTTCTTTAATCCCCGTCGTCCATCTCGTGCCTTTGCCGTTGCTACTTGCATTG
GTAGGGCATCATCAGTCAGCCAGTTTACACGTCGCACCAAAAACACACGAACATT
CAGATCAGCTAGTAGCTTGAGAGTGAGAGGGTCCCCCCGTCGTCTCTGCCTTCTT
GCGCTGCATCTTTCCGGGCGGGACATCTGGTGCTCTTGTCCGCTAATGGCCCCC
CACCCCCTCCTTTCTTTTTATTCCAAACCCGCTCCTCCCCTGCTTTCCCTCTCTTAT
TCAAACTCGCAGTCACAGCCTCCCTCTTTTTCTAACGCCACCGGCCGTTGCCACC
CCTCATCACACCCTCACTTCAGCACTTCAGCTGCAGCTCAGTACCGCTAGGCTTG
GAGCGCGCACCGGCGCGGAGCAGACAGCGGAGCCTCTCTTGCCGTCCGGCTGC
GAATTCCGGGGATGGGCAAGGCGGCGCGGTGGTTCCGCAGCTTGCTTGGCGGC
GGGAGGAAGGACCAGGAGAGGCAGCGGGCCTCGCCGGCGCCGCCGCCCACCG
CGGACAGGAAGCGCTGGAGCTTCGCTCGCTCGTCGCGGGACTCGGCCGAGGCC
GCGGCGGCGGCGACCGAGGGCTCCGTGCGGGGCGGTGCCGCCGCCGCCGGCG
GTAACGCGGCGATCGCGAGGGCGGCCGAGGCCGCGTGGCTCAAGTCGCTCTAC
GACGACACGGGGCGGCAGCAGAGCAAGCACGCCATCGCCGTCGCTGCGGCTAC
CGCGGCGGCGGCGGACGCGGCCGTGGCCGCCGCGCAGGCCGCCGTCGAGGTC
GTCCGGCTCACCAGCCAGGGCCCTGTCTTCGGCGGCGGAGGTGGAGGAGGGGC
CGTGCTCGACCCCCGTGGCCGCGCCGGCGCCGCCGTCAAGATCCAGACGGCCT
TCAGAGGCTTCTTGGCGAAGAAGGCGCTGCGAGCGCTCAAGGCGCTGGTGAAGC
TGCAGGCGCTGGTGCGCGGCTACCTGGTGCGGCGGCAGGCGGCGGCGACGCT
GCAGAGCATGCAGGCGCTGGTCCGCGCGCAGGCCACCGTCCGCGCCGCCCGCG
GCTGCCGCGCCCTGCCCTCGCTCCCGCCGCTCCACCACCCAGCTGCATTCCGCC
CGCGCTTCTCGCTGCAAGAGCGGTACGCTGACGACACGCGCAGCGAGCACGGC
GTGGCGGCGTACAGCCGGCGCCTGTCCGCGAGCATCGAGTCGGCGTCGTACGG
GGGCGGCGGGTACGACCGGAGCCCCAAGATCGTGGAGATGGACACGGCGCGGC
CGAGGTCCCGCGCGTCGTCCCTTCGCACCGAAGACGAGTGGTACGCGCAGTCG
GTGTCGTCGCCGCTGCAGCCGCCGTGCCACCACCTGCCGCCGCGCATCGCGGT
GCCGACGTCGCGCCACTTCCCGGACTACGACTGGTGCGCGCCGGAGAAGCCCC
GGCCGGCGACGGCGCAGTGCACGCCCCGGTTCGCGCCGCCGACCCCGGCAAA
GAGCGTCTGCGGCGGCGGCGGTGGTAACGGCGGCTACTACGCCCACCACCTCG
CCGCGGGGTCGCCCAACTGCCCCGGGTACATGTCGAGCACGCAGTCGTCGGAG
GCCAAGTCGTCGTCCCGGTCGCACAGCGCGCCGAAGCAGCGGCCGCCGGAGCA
GCAGCAGCCGTCCCGGAAGCGCGTGCCGCTGAGCGAGGTGGTCCTGGAGGCCC
GCGCCAGCCTGGGCGGCGTCGGCGTCGGTATGATGCACAAGCCGTGCAACACC
CGCGCCGCGCAGCCGCAGGAGCCGTTCGATTTCCGCGCCGCCGTCGTCAGCCG
GTTCGAGCAGCGCGCGTCGGACGCCGCTGCCGCCGCCGAACGGGACCGCGACG
TGTTGTTCCTGCAGAGGAGGTGGTGAAGGTGAACCGACCGATCGATCGATCGGT
CAGTGAGTTAGTCGAAGTGCTCCGCCTGCCTGAGTGAGATTATGGCCTAGTATGA
TTAATCGGTGCTGCTAATAGGGATTGTTAATTAGGTTCTCATTAATTCCTCGCCCTT
TTGTGATCTCTGTTAGTTCTTCCGATCGCGTCCATGACCTCTCTCTGCAGTCGGCC
ATTCTTCCTGCTTGCAATAAGCTAGTGTGTGTGTGTGTGTGAAGTAGGGAGAGATT
TCATCATCCCGTTCCGTTTCGTTTTTTTCGTTCATAAAAACAGTAGTGCAGCTGGAT
CATCAGCTCGATGTACATTCCGCCTGTTCTCCGATCATCACCATCAAGAAAGAGA
GAAAAAAAA
SEQ ID NO: 62; CDS:
ATGGGCAAGGCGGCGCGGTGGTTCCGCAGCTTGCTTGGCGGCGGGAGGAAGGA
CCAGGAGAGGCAGCGGGCCTCGCCGGCGCCGCCGCCCACCGCGGACAGGAAG
CGCTGGAGCTTCGCTCGCTCGTCGCGGGACTCGGCCGAGGCCGCGGCGGCGGC
GACCGAGGGCTCCGTGCGGGGCGGTGCCGCCGCCGCCGGCGGTAACGCGGCG
ATCGCGAGGGCGGCCGAGGCCGCGTGGCTCAAGTCGCTCTACGACGACACGGG
GCGGCAGCAGAGCAAGCACGCCATCGCCGTCGCTGCGGCTACCGCGGCGGCGG
CGGACGCGGCCGTGGCCGCCGCGCAGGCCGCCGTCGAGGTCGTCCGGCTCAC
CAGCCAGGGCCCTGTCTTCGGCGGCGGAGGTGGAGGAGGGGCCGTGCTCGACC
CCCGTGGCCGCGCCGGCGCCGCCGTCAAGATCCAGACGGCCTTCAGAGGCTTC
TTGGCGAAGAAGGCGCTGCGAGCGCTCAAGGCGCTGGTGAAGCTGCAGGCGCT
GGTGCGCGGCTACCTGGTGCGGCGGCAGGCGGCGGCGACGCTGCAGAGCATG
CAGGCGCTGGTCCGCGCGCAGGCCACCGTCCGCGCCGCCCGCGGCTGCCGCG
CCCTGCCCTCGCTCCCGCCGCTCCACCACCCAGCTGCATTCCGCCCGCGCTTCT
CGCTGCAAGAGCGGTACGCTGACGACACGCGCAGCGAGCACGGCGTGGCGGCG
TACAGCCGGCGCCTGTCCGCGAGCATCGAGTCGGCGTCGTACGGGGGCGGCGG
GTACGACCGGAGCCCCAAGATCGTGGAGATGGACACGGCGCGGCCGAGGTCCC
GCGCGTCGTCCCTTCGCACCGAAGACGAGTGGTACGCGCAGTCGGTGTCGTCGC
CGCTGCAGCCGCCGTGCCACCACCTGCCGCCGCGCATCGCGGTGCCGACGTCG
CGCCACTTCCCGGACTACGACTGGTGCGCGCCGGAGAAGCCCCGGCCGGCGAC
GGCGCAGTGCACGCCCCGGTTCGCGCCGCCGACCCCGGCAAAGAGCGTCTGCG
GCGGCGGCGGTGGTAACGGCGGCTACTACGCCCACCACCTCGCCGCGGGGTCG
CCCAACTGCCCCGGGTACATGTCGAGCACGCAGTCGTCGGAGGCCAAGTCGTCG
TCCCGGTCGCACAGCGCGCCGAAGCAGCGGCCGCCGGAGCAGCAGCAGCCGTC
CCGGAAGCGCGTGCCGCTGAGCGAGGTGGTCCTGGAGGCCCGCGCCAGCCTGG
GCGGCGTCGGCGTCGGTATGATGCACAAGCCGTGCAACACCCGCGCCGCGCAG
CCGCAGGAGCCGTTCGATTTCCGCGCCGCCGTCGTCAGCCGGTTCGAGCAGCG
CGCGTCGGACGCCGCTGCCGCCGCCGAACGGGACCGCGACGTGTTGTTCCTGC
AGAGGAGGTGGTGA
SEQ ID NO: 63; protein
MGKAARWFRSLLGGGRKDQERQRASPAPPPTADRKRWSFARSSRDSAEAAAAATE
GSVRGGAAAAGGNAAIARAAEAAWLKSLYDDTGRQQSKHAIAVAAATAAAADAAVAA
AQAAVEVVRLTSQGPVFGGGGGGGAVLDPRGRAGAAVKIQTAFRGFLAKKALRALK
ALVKLQALVRGYLVRRQAAATLQSMQALVRAQATVRAARGCRALPSLPPLHHPAAFR
PRFSLQERYADDTRSEHGVAAYSRRLSASIESASYGGGGYDRSPKIVEMDTARPRSR
ASSLRTEDEWYAQSVSSPLQPPCHHLPPRIAVPTSRHFPDYDWCAPEKPRPATAQCT
PRFAPPTPAKSVCGGGGGNGGYYAHHLAAGSPNCPGYMSSTQSSEAKSSSRSHSA
PKQRPPEQQQPSRKRVPLSEVVLEARASLGGVGVGMMHKPCNTRAAQPQEPFDFR
AAVVSRFEQRASDAAAAAERDRDVLFLQRRW
MEDICAGO TRUNCATULA
SEQ ID NO: 64; MTR_8G102400; Genomic Sequence:
CTCAAACACTACAACCAATGGGTAGAACCATAAGGTGGTTCAAGAGTTTGTTTGG
GATAAAGAAAGACAGAGATAATTCAAACTCAAATTCTTCAAGTACCAAATGGAATC
CTTCTCTTCCTCATCCTCCTTCTCAAGATTTCTCAAAGAGAGATTCGAGAGGCTTG
TGTCATAATCCAGCTACCATACCTCCCAACATTTCACCTGCAGAAGCTGCTTGGGT
TCAATCCTTCTACTCAGAAACTGAGAAGGAGCAAAACAAGCACGCCATTGCGGTA
GCAGCTGCAACAGCAGCAGCCGCAGATGCTGCTGTGGCAGCTGCTCAAGCTGCC
GTGGGCTGTGGTTAGATTAACCAGCCACGGCAGAGACACCATGTTTGGTGGTGG
ACACCAGAAATTTGCTGCTGTCAAGATTCAAACAACATTTAGGGGTTACTTGGTAA
GTTTGATTCACTTTTCTTTAATTAATTATGTGATTTTACTAATGCAGTTCTAAGAAAA
ACAGATTTTGCTCAAATTGAACTAGTCAGATTCAAATTCTAGGCTTTTTATGTTTTA
AAGTATTATTATAGCTTCAATTAGAACTAAAGAAAGTGTTAATGAACTTGGTTTGAT
GTTTATATATTTCTATTTTTCTTGTGGCATTGTGGAATGTGAGAGAATTTGAAATTG
GTTTTTCTATAATAGGCAAGAAAAGCACTAAGAGCCTTAAAGGGATTGGTAAAGTT
ACAAGCACTAGTGAGAGGGTACTTAGTGAGGAAGCAAGCAACAGCAACATTACAC
AGTATGCAAGCTCTAATTAGAGCACAAGCAACAGTAAGGTCTCATAAATCTCGTGG
ACTCATCATAAGCACAAAGAATGAAACAAATAACAGATTTCAAACACAAGCTAGAA
GATCCACGGTAAAATACATAAACAGATCTCATATTTTAATTCCTAGTATCACTTGAA
TATTTACATTTCTTATGATTGTTATTTTGCAGGAAAGGTATAATCACAATGAGAGTA
ACAGGAACGAGTACACAGCTTCAATTCCTATTCACAGCAGAAGATTATCATCATCT
TTTGATGCTACAATGAACAGTTATGATATTGGAAGTCCAAAAATAGTAGAAGTTGAT
ACTGGAAGACCAAAATCAAGGTCTAGAAGAAGCAATACATCAATTTCAGATTTTGG
AGATGACCCTTCATTTCAAACACTTTCTTCTCCACTTCAAGTTACTCCATCTCAGTT
ATACATTCCAAATCAAAGAAATTATAACGAATCAGATTGGGGAATAACAGGTGAAG
AATGCAGATTTTCAACTGCACAGAGCACTCCACGTTTCACAAGTTCATGTAGTTGT
GGATTTGTTGCACCTTCCACACCTAAAACAATTTGTGGAGATAGTTTTTACATTGGT
GATTATGGTAATTATCCTAATTACATGGCTAATACACAGTCTTTTAAGGCTAAATTG
AGGTCTCATAGTGCTCCAAAGCAACGACCTGAACCAGGTCCGAAGAAGAGGCTTT
CATTGAATGAATTGATGGAATCTAGAAACAGTTTGAGTGGAGTTAGAATGCAGAGG
TCTTGTTCACAGATTCAGGATGCTATTAATTTTAAGAATGCTGTGATGAGTAAACTT
GATAAGTCCACTGATTTTGATAGAAACTTTTCAAAGCAGAGGAGGTTGTGATCCGA
GGAGCAATGCCGTGTGTCCGGTGTCCATGTCAGAGTCCATGCTTCAAAGTGGTGA
TTGATTAGTGAGTTTAAGAAGCATTTATGAGAGCGCTAGTAAATTGATTGGTAATTT
GCAGACATATAGTGGGTAGGGAACAGGTAAGTGAAGACAAAATTGAAGGATAATT
AATAACAATGATAAACTACAAGTTTGGTGATGGAATTTG
SEQ ID NO: 65; CDS:
ATGGGTAGAACCATAAGGTGGTTCAAGAGTTTGTTTGGGATAAAGAAAGACAGAG
ATAATTCAAACTCAAATTCTTCAAGTACCAAATGGAATCCTTCTCTTCCTCATCCTC
CTTCTCAAGATTTCTCAAAGAGAGATTCGAGAGGCTTGTGTCATAATCCAGCTACC
ATACCTCCCAACATTTCACCTGCAGAAGCTGCTTGGGTTCAATCCTTCTACTCAGA
AACTGAGAAGGAGCAAAACAAGCACGCCATTGCGGTAGCAGCTCTGCCGTGGGC
TGTGGTTAGATTAACCAGCCACGGCAGAGACACCATGTTTGGTGGTGGACACCAG
AAATTTGCTGCTGTCAAGATTCAAACAACATTTAGGGGTTACTTGGCAAGAAAAGC
ACTAAGAGCCTTAAAGGGATTGGTAAAGTTACAAGCACTAGTGAGAGGGTACTTA
GTGAGGAAGCAAGCAACAGCAACATTACACAGTATGCAAGCTCTAATTAGAGCAC
AAGCAACAGTAAGGTCTCATAAATCTCGTGGACTCATCATAAGCACAAAGAATGAA
ACAAATAACAGATTTCAAACACAAGCTAGAAGATCCACGGAAAGGTATAATCACAA
TGAGAGTAACAGGAACGAGTACACAGCTTCAATTCCTATTCACAGCAGAAGATTAT
CATCATCTTTTGATGCTACAATGAACAGTTATGATATTGGAAGTCCAAAAATAGTAG
AAGTTGATACTGGAAGACCAAAATCAAGGTCTAGAAGAAGCAATACATCAATTTCA
GATTTTGGAGATGACCCTTCATTTCAAACACTTTCTTCTCCACTTCAAGTTACTCCA
TCTCAGTTATACATTCCAAATCAAAGAAATTATAACGAATCAGATTGGGGAATAACA
GGTGAAGAATGCAGATTTTCAACTGCACAGAGCACTCCACGTTTCACAAGTTCATG
TAGTTGTGGATTTGTTGCACCTTCCACACCTAAAACAATTTGTGGAGATAGTTTTTA
CATTGGTGATTATGGTAATTATCCTAATTACATGGCTAATACACAGTCTTTTAAGGC
TAAATTGAGGTCTCATAGTGCTCCAAAGCAACGACCTGAACCAGGTCCGAAGAAG
AGGCTTTCATTGAATGAATTGATGGAATCTAGAAACAGTTTGAGTGGAGTTAGAAT
GCAGAGGTCTTGTTCACAGATTCAGGATGCTATTAATTTTAAGAATGCTGTGATGA
GTAAACTTGATAAGTCCACTGATTTTGATAGAAACTTTTCAAAGCAGAGGAGGTTG
TGA
SEQ ID NO: 66; Protein:
MGRTIRWFKSLFGIKKDRDNSNSNSSSTKWNPSLPHPPSQDFSKRDSRGLCHNPATI
PPNISPAEAAWVQSFYSETEKEQNKHAIAVAALPWAVVRLTSHGRDTMFGGGHQKFA
AVKIQTTFRGYLARKALRALKGLVKLQALVRGYLVRKQATATLHSMQALIRAQATVRS
HKSRGLIISTKNETNNRFQTQARRSTERYNHNESNRNEYTASIPIHSRRLSSSFDATM
NSYDIGSPKIVEVDTGRPKSRSRRSNTSISDFGDDPSFQTLSSPLQVTPSQLYIPNQR
NYNESDWGITGEECRFSTAQSTPRFTSSCSCGFVAPSTPKTICGDSFYIGDYGNYPN
YMANTQSFKAKLRSHSAPKQRPEPGPKKRLSLNELMESRNSLSGVRMQRSCSQIQD
AINFKNAVMSKLDKSTDFDRNFSKQRRL
Triticum aestivum
SEQ ID NO: 67; TRAES_3BF002600110CFD_c1; genomic Sequence:
ATGGGCAAGGCGGCGAGGTGGCTGCGTGGCTTGCTGGGCGGCGGCGGCAAGAA
GGAGCAGGGGAAGGAGCAGAGGCGCCCGGCCACGGCGCCGCACGGGGACAGG
AAGCGCTGGAGCTTCTGCAAGTCCACCAGGGACTCGGCAGAGGCGGAGGCGGC
GGCCGCGGCCGCGGCGCTCAGCGGCAACGCGGCGATCGCGCGCGCGGCCGAG
GCGGCATGGCTCAAGTCCTTGTACAACGAGACCGAGCGCGAGCAGAGCAAGCAC
GCCATCGCCGTCGCCGCGGCCACCGCGGCGGCGGCGGACGCGGCTATGGCTG
CCGCACAGGCAGCCGTGGAGGTCGTGCGGCTCACCAGCAAAGGGCCGACGTCG
ACGGTGCTCGCCGACGCCGTCGCGGAGCCCCACGGCCGTGCCTCCGCCGCGGT
CAAGATCCAGACGGCGTTCCGTGGCTTCCTGGTGAGTAATTTCCTTCCTAACAGC
GGCGCCATGATTTCCGCAGGTTTAAGCGCTGAGTAACCAAATCAATGCGTGTTGA
ATTATCGCAGGCCAAGAAGGCTCTGCGCGCGCTCAAGGGGCTGGTGAAGCTGCA
GGCGCTGGTGCGCGGCTACCTGGTGCGGAAGCAGGCGGCGGCCACGCTGCAGA
GCATGCAGGCGCTCGTCCGCGCGCAGGCCTGCATCCGCGCTGCCCGCTCGCGC
GCCGCGGCGCTCCCGACGAACCTTCGCGTCCACCCCACTCCTGTCCGGCCGCG
CTACTCGTTGGTAAGTGACCACGGGTCCACGGCCGGCATCGCTTGCGACCAAAG
CAATCGATCTCAATGTCTCTGACCGTCCGAGGTCGCGTTGTTCTAGCTAGCCGAC
CGTAACAAATGTGCGCGTGCGTGGTTTCTTGCTTGTGTCTGCAGCAAGAGCGGTA
CAGCACCACGGAGGATTCCCGGAGCGACCACCGCGTGGCGCCGTACTACAGCC
GCCGGCTGTCGGCGAGCGTGGAGTCGTCGTCGTGCTACGGCTACGACCGGAGC
CCCAAGATCGTGGAGATGGACACCGGCCGGCCCAAGTCGCGCTCCTCCTCGCTC
CGGACGACCTCCCCCGGCGCCAGCGAGGAGTGCTACGCCCACTCGGTGTCGTC
GCCGCTCATGCCGTGCCGAGCGCCCCCGCGGATCGCGGCGCCCACCGCGCGCC
ACTTCCCGGAGTACGAGTGGTGCGAGAAGGCCCGGCCGGCGACGGCGCAGAGC
ACGCCCCGGTACACGAGCTACGCGCCGGTGACGCCGACCAAGAGCGTGTGCGG
CGGCTACACCTACAGCAACAGCCCGTCGACGCTCAACTGCCCCAGCTACATGTC
GAGCACGCAGTCGTCCGTGGCGAAGGTGCGTTCGCAGAGCGCGCCGAAGCAGC
GGCCGGAGGAGGGCGCGGTACGGAAGAGGGTGCCGCTGAGCGAGGTGATCATC
CTGCAGGAGGCCCGGGCGAGCCTGGGCGGCGGCGGGGGCACGCAGAGGTCGT
GCAACCGGCCGGCGCAGGAGGAGGCGTTCAGCTTCAAGAAGGCCGTCGTGAGC
CGCTTCGACCGCTCGTCGGAGGCGGCCGAGAGGGAACGTGACCGGGACCGGGA
CTTGTTCCTGCAGAAGGGGTGGTGA
SEQ ID NO: 68; CDS:
ATGGGCAAGGCGGCGAGGTGGCTGCGTGGCTTGCTGGGCGGCGGCGGCAAGAA
GGAGCAGGGGAAGGAGCAGAGGCGCCCGGCCACGGCGCCGCACGGGGACAGG
AAGCGCTGGAGCTTCTGCAAGTCCACCAGGGACTCGGCAGAGGCGGAGGCGGC
GGCCGCGGCCGCGGCGCTCAGCGGCAACGCGGCGATCGCGCGCGCGGCCGAG
GCGGCATGGCTCAAGTCCTTGTACAACGAGACCGAGCGCGAGCAGAGCAAGCAC
GCCATCGCCGTCGCCGCGGCCACCGCGGCGGCGGCGGACGCGGCTATGGCTG
CCGCACAGGCAGCCGTGGAGGTCGTGCGGCTCACCAGCAAAGGGCCGACGTCG
ACGGTGCTCGCCGACGCCGTCGCGGAGCCCCACGGCCGTGCCTCCGCCGCGGT
CAAGATCCAGACGGCGTTCCGTGGCTTCCTGGCCAAGAAGGCTCTGCGCGCGCT
CAAGGGGCTGGTGAAGCTGCAGGCGCTGGTGCGCGGCTACCTGGTGCGGAAGC
AGGCGGCGGCCACGCTGCAGAGCATGCAGGCGCTCGTCCGCGCGCAGGCCTGC
ATCCGCGCTGCCCGCTCGCGCGCCGCGGCGCTCCCGACGAACCTTCGCGTCCA
CCCCACTCCTGTCCGGCCGCGCTACTCGTTGCAAGAGCGGTACAGCACCACGGA
GGATTCCCGGAGCGACCACCGCGTGGCGCCGTACTACAGCCGCCGGCTGTCGG
CGAGCGTGGAGTCGTCGTCGTGCTACGGCTACGACCGGAGCCCCAAGATCGTGG
AGATGGACACCGGCCGGCCCAAGTCGCGCTCCTCCTCGCTCCGGACGACCTCCC
CCGGCGCCAGCGAGGAGTGCTACGCCCACTCGGTGTCGTCGCCGCTCATGCCG
TGCCGAGCGCCCCCGCGGATCGCGGCGCCCACCGCGCGCCACTTCCCGGAGTA
CGAGTGGTGCGAGAAGGCCCGGCCGGCGACGGCGCAGAGCACGCCCCGGTACA
CGAGCTACGCGCCGGTGACGCCGACCAAGAGCGTGTGCGGCGGCTACACCTAC
AGCAACAGCCCGTCGACGCTCAACTGCCCCAGCTACATGTCGAGCACGCAGTCG
TCCGTGGCGAAGGTGCGTTCGCAGAGCGCGCCGAAGCAGCGGCCGGAGGAGGG
CGCGGTACGGAAGAGGGTGCCGCTGAGCGAGGTGATCATCCTGCAGGAGGCCC
GGGCGAGCCTGGGCGGCGGCGGGGGCACGCAGAGGTCGTGCAACCGGCCGGC
GCAGGAGGAGGCGTTCAGCTTCAAGAAGGCCGTCGTGAGCCGCTTCGACCGCTC
GTCGGAGGCGGCCGAGAGGGAACGTGACCGGGACCGGGACTTGTTCCTGCAGA
AGGGGTGGTGA
SEQ ID NO: 69 Protein:
MGKAARWLRGLLGGGGKKEQGKEQRRPATAPHGDRKRWSFCKSTRDSAEAEAAAA
AAALSGNAAIARAAEAAWLKSLYNETEREQSKHAIAVAAATAAAADAAMAAAQAAVEV
VRLTSKGPTSTVLADAVAEPHGRASAAVKIQTAFRGFLAKKALRALKGLVKLQALVRG
YLVRKQAAATLQSMQALVRAQACIRAARSRAAALPTNLRVHPTPVRPRYSLQERYST
TEDSRSDHRVAPYYSRRLSASVESSSCYGYDRSPKIVEMDTGRPKSRSSSLRTTSPG
ASEECYAHSVSSPLMPCRAPPRIAAPTARHFPEYEWCEKARPATAQSTPRYTSYAPV
TPTKSVCGGYTYSNSPSTLNCPSYMSSTQSSVAKVRSQSAPKQRPEEGAVRKRVPL
SEVIILQEARASLGGGGGTQRSCNRPAQEEAFSFKKAVVSRFDRSSEAAERERDRDR
DLFLQKGW
GLYCINE MAX
SEQ ID NO: 70 GLYMA_08G200400; Genomic Sequence:
GGAGAGCTCCCTTTCTTTGAACCCTTTTTACTCAATGCCCGGTTTGAACCAAGCTC
ACGGGATTGTTGTCAGTGGGTATCTTTCATATGTACTACACGTTGCTCCAGAATGT
TAGAGAGAGATAGATTTTTTTTATAATCAATAGTTTAAGTGATGTGGATGCTAAGGA
TTCATGAGGAACGTACCTTAAAGACTCGCTGAGTACTTATATAATTACTTATAAATT
ATTTACTAATAAGATTCTTATATTGATAATAAAATTTAAATTTACATTTTTGTATAATT
TGTGTTCGATCCTTACCTGATAAATACATAGAGCAACAAAGTAATAATATTGCATTT
TTTTTAGCATAATAACATAGTGCAAAGTGAGATAAAGAGAGAAAGAGGAATTGGTT
ATTGTTGGTAGTACTTTGGTAGTGTTATTGGAGTGAGTGGGGTGGGAAGGGAATT
CGGCGCTCTTTCTCTTTTTTGAATTCAAGATCAGGTAAGAAGCTGTTTGTTTTGTTT
TGGGTGTTGGGGTTGGGGGGCTTTTCCCTATGATTATTATTGTCACTTATTTCATA
GGATTTCACCTTTGTGCTTTGTCTGTAATTCTTGTCTCTTTACTCAAGAGGGGGTG
AGATTTTGAAGCGTAATGGTTACTATTTTAATGTTGTTTTTTTTTTTCATTTCATTTG
AGCCTCTGCTAACTTTCTGTGACCGCTTTTTGTCCTCTAATGCACTTCTTCACGAG
GTCCTGTCAGAGGTTTATTTTCAAAAAATGTTCCATCTTTATCCATTTTTTCGCTCC
CCTCACTATAATTTTTAAGGTTTGTTTGTTTGATGCCTTTCATTTTACCCCGTTTTAT
TAATCTTTTCAACCATCCTGCTCTCATCACTTCTCTTCTGCTAGATTCTTTACACTTT
ACATTACACCCTCTTTTGTTTCTTTGTCCCTCCAATTTTTAAAACCAAGAATTTTCAT
TTTCACCTGTCCCTTGTATATTGAACCATCTGTTTTCACTTGGTTGCTACACTTGTT
CACTTACACAAGCAAAACCCCTCTTTATCTTTAACCAAAAGAGGCAAAGTTTGGCT
TAACTTTGGCACCGTTTTCTCATTCCAGATCGTGACTGAAAAAGTTGTGTGAAGTT
ATTATTATGGGGAAAGCTAGCAGGTGGTTGAAGGGGTTGTTGGGGATGAAGAAG
AAAGGTGGAGTTTTGCCAAGCCACCACCTTCTTCAGTTCCTGCCACTGATAACAAC
AACACCTGGCTCAGATCCTATATTTCTGAGACAGAGAATGAGCAGAACAAGCATG
CAATTGCCGTGGCAGCCGCCACCGCTGCTGCTGCCGATGCCGCCGTGGCCGCC
GCACAGGCGGCCGTGGCTGTTGTGAGGCTCACAAGCCAGGGGAGAGGGGCATT
GTTCAGTGGAAGCAGGGAGAAATGGGCTGCTGTGAAGATCCAAACTTTTTTTAGA
GGCTACTTGGTATGTGTCTTGTCTTTTTGTGATGTTTCAAATCAAAATTTTGGTGTT
GTTTATGTGGGTATGTATGTGTGTGTGTGTCTGCTTTCTTTCATTTTGAAAATGTTG
TTTGGTTAATTGCATTGGTTCTTGTCTTGGACATATTTATTTTATTAGGTTTGTTTAA
GTGAAGTGTTTTGCTTGATTCCTACTCCTTTGTCTCCTCTCAAAAATTATTTTAGAT
TCTTAATGAAACAGTGCCTTTCACTTTCTGTTTGGGTCATTCAAACTACCCTTTGCA
CTTTCCATTCTCCACGTTAGAGTTGACTTGTCTTGTTATTTTGATGCTCTTTTAATTA
AACCATTTTCAATATTTGACAATTCTTTATGTACATTTTGTCAATTTCATGGTTTTAT
TAAGTTCCATTACTATACTAGCACTTTTAATTTAAACTTTTATGTTGCTTACTTTCAG
GCACGGAAGGCTCTTAGAGCACTGAAAGGATTGGTTAAGATACAAGCTCTTGTTA
GAGGGTATTTGGTTAGAAAGAGGGCTGCTGCAACTCTTCACAGTATGCAAGCTCT
AATAAGAGCTCAAACTGCTGTTAGAACACAGCGAGCTCGTCGTTCCATGAGCAAA
GAAGACAGATTTCTACCTGAAGTTCTTGCAAGAAAACCTGTGGTAATTAAAAACTG
GTTTTCTAGTCCTGAATGATTACCAACTTCACTCATTTTATTTTATCTATCAGCAAAC
AATGTCTTATTTGCTTGGTGTTGTGTCCTTTTTGTACTTTTTAGGAACGATTTGATG
AAACAAGGAGTGAATTCCACAGTAAAAGGCTACCTACATCCTATGAAACATCCTTG
AATGGTTTTGATGAGAGCCCCAAGATTGTTGAAATTGACACATACAAGACTCGATC
GAGGTCTAGGCGCTTCACCTCTACAATGTCTGAGTGTGGAGAAGACATGTCCTGC
CATGCAATCTCATCCCCTCTTCCTTGTCCGGTCCCCGGTCGAATCTCGGTTCCTG
ATTGCAGACACATTCAAGATTTTGATTGGTACTACAACGTGGATGAGTGTAGATTC
TCCACTGCTCATAGCACCCCGCGTTTCACAAACTATGTGAGGGCTAATGCTCCAG
CTACACCAGCCAAGAGTGTTTGTGGAGACACTTTCTTCAGACCTTGCTCCAATTTC
CCCAACTACATGGCCAATACTCAGTCATTCAATGCAAAACTAAGGTCTCACAGTGC
TCCAAAGCAAAGACCTGAACCCAAGAAAAGGCTCTCACTCAATGAAATGATGGCA
GCAAGAAACAGCATAAGTGGTGTTAGAATGCAAAGGCCATCATCCAATTTCTTTCC
AGACTCAAGAAGAATCCTGGAATTTTTTACAATCACAAGGAATATTTGAGAGGCAA
TTGGAGTCTAAATGATGTTAGTAATATCTAGAATTTGTTTTTTTTTTTTTCACTTGTG
CTTACAACTTACAAATTCAGGGATGAAATTCTCAATTCTCAATTCTGCTTGTGTACA
TTCTTTTAATTAAAGAGTTTTTTTTTTTTTTTT
SEQ ID NO: 71; CDS:
ATGGGGAAAGCTAGCAGGTGGTTGAAGGGGTTGTTGGGGATGAAGAAGGAGAAG
GACCACAGTGACAATTCAGGCTCATTGGCTCCTGACAAGAAGGAGAAGAAAAGGT
GGAGTTTTGCCAAGCCACCACCTTCTTCAGTTCCTGCCACTGATAACAACAACACC
TGGCTCAGATCCTATATTTCTGAGACAGAGAATGAGCAGAACAAGCATGCAATTG
CCGTGGCAGCCGCCACCGCTGCTGCTGCCGATGCCGCCGTGGCCGCCGCACAG
GCGGCCGTGGCTGTTGTGAGGCTCACAAGCCAGGGGAGAGGGGCATTGTTCAGT
GGAAGCAGGGAGAAATGGGCTGCTGTGAAGATCCAAACTTTTTTTAGAGGCTACT
TGGCACGGAAGGCTCTTAGAGCACTGAAAGGATTGGTTAAGATACAAGCTCTTGT
TAGAGGGTATTTGGTTAGAAAGAGGGCTGCTGCAACTCTTCACAGTATGCAAGCT
CTAATAAGAGCTCAAACTGCTGTTAGAACACAGCGAGCTCGTCGTTCCATGAGCA
AAGAAGACAGATTTCTACCTGAAGTTCTTGCAAGAAAACCTGTGGAACGATTTGAT
GAAACAAGGAGTGAATTCCACAGTAAAAGGCTACCTACATCCTATGAAACATCCTT
GAATGGTTTTGATGAGAGCCCCAAGATTGTTGAAATTGACACATACAAGACTCGAT
CGAGGTCTAGGCGCTTCACCTCTACAATGTCTGAGTGTGGAGAAGACATGTCCTG
CCATGCAATCTCATCCCCTCTTCCTTGTCCGGTCCCCGGTCGAATCTCGGTTCCT
GATTGCAGACACATTCAAGATTTTGATTGGTACTACAACGTGGATGAGTGTAGATT
CTCCACTGCTCATAGCACCCCGCGTTTCACAAACTATGTGAGGGCTAATGCTCCA
GCTACACCAGCCAAGAGTGTTTGTGGAGACACTTTCTTCAGACCTTGCTCCAATTT
CCCCAACTACATGGCCAATACTCAGTCATTCAATGCAAAACTAAGGTCTCACAGTG
CTCCAAAGCAAAGACCTGAACCCAAGAAAAGGCTCTCACTCAATGAAATGATGGC
AGCAAGAAACAGCATAAGTGGTGTTAGAATGCAAAGGCCATCATCCAATTTCTTTC
CAGACTCAAGAAGAATCCTGGAATTTTTTACAATCACAAGGAATATTTGA
SEQ ID NO: 72; Protein:
MGKASRWLKGLLGMKKEKDHSDNSGSLAPDKKEKKRWSFAKPPPSSVPATDNNNT
WLRSYISETENEQNKHAIAVAAATAAAADAAVAAAQAAVAVVRLTSQGRGALFSGSR
EKWAAVKIQTFFRGYLARKALRALKGLVKIQALVRGYLVRKRAAATLHSMQALIRAQT
AVRTQRARRSMSKEDRFLPEVLARKPVERFDETRSEFHSKRLPTSYETSLNGFDESP
KIVEIDTYKTRSRSRRFTSTMSECGEDMSCHAISSPLPCPVPGRISVPDCRHIQDFDW
YYNVDECRFSTAHSTPRFTNYVRANAPATPAKSVCGDTFFRPCSNFPNYMANTQSF
NAKLRSHSAPKQRPEPKKRLSLNEMMAARNSISGVRMQRPSSNFFPDSRRILEFFTIT
RNI
Arabidopsis
SEQ ID NO: 73; AT3G16490, IQ-DOMAIN 26, IQD26;
Genomic Sequence:
ATATATAAATGGTGATACTTATATTTATTTTTGAAGAACCTATCCATCACATTCTCTC
TCTCTTTTCTTGAACTTCTTATCTCTCTTTTTTTTTACAGTTCTCCTTTCTCAAAACAT
CACATTGTGTCTTCAGTTTCAGTGCCGTAAATTTTGTTTTCCTCTTCATTTTCTGCA
TACGAAAAGTTCTTTTGGGTATTTGTGATTTGTCATCTCCGAAACGTTTCTCTCTTT
AAAACTTTTTTTGACCGTTCTTTATAATTTGAATTGAAAGAGAAGATGGGAAGAGCT
GCGAGATGGTTCAAGGGTATTTTTGGTATGAAGAAGAGCAAAGAGAAAGAGAACT
GTGTTTCCGGCGACGTTGGAGGTGAAGCCGGTGGTTCTAACATTCACCGGAAAGT
TCTCCAAGCTGACTCCGTCTGGCTCAGAACTTACCTTGCGGAAACAGACAAAGAA
CAGAACAAACACGCGATTGCGGTTGCTGCTGCTACAGCCGCGGCTGCTGACGCA
GCGGTTGCAGCGGCTCAAGCTGCTGTGGCGGTGGTCAGGTTAACAAGTAACGGA
AGAAGCGGAGGATATTCCGGGAACGCAATGGAGCGGTGGGCCGCAGTGAAAATT
CAATCAGTCTTCAAGGGCTATTTGGTAAATTTCTTAAAAACCTCCAAAACACTTTTT
TTTTTTTTTGGTGTGTAATCGATTTCGACGCAAAAAGATTGAATTTTGCCATGTGGG
TATCGTTTAGATTCGACAAAACTAAAAGAAAGTATGTCTGTACTTTACTTGCTCCTT
ACACATCTTCGTTAATGAAACAGTACAACACATCTTTGCTAATTTCAAGAAAGTTAG
ATTCTTTTCTGATTAAACACTAAAATGTGGTTATTTGGTCTAAGTATTTTTTTTTTTTT
TGTTATAGGCGAGAAAAGCGTTACGAGCTTTGAAAGGTTTAGTGAAGCTACAAGC
TTTGGTAAGAGGATACTTAGTCCGCAAACGCGCCGCCGAAACGCTGCATAGCATG
CAAGCTCTCATTAGAGCTCAAACCAGCGTCCGATCGCAACGCATCAACCGCAACA
ACATGTTTCATCCTCGACACTCACTTGTAATAACCATTCTTTTTCTTTTGTCTTATTT
CTAACATAATCTCTATAGTCAAAACTTATGTTTTGATGGATTGATTTGATTATAGGA
GAGGTTGGATGATTCAAGAAGTGAAATCCATAGCAAGAGAATATCAATCTCTGTAG
AGAAACAGAGTAATCACAACAACAATGCGTACGATGAGACCAGTCCCAAGATTGT
GGAGATTGATACTTACAAGACGAAATCAAGATCAAAGAGAATGAATGTGGCTGTAT
CCGAATGTGGAGATGATTTCATCTATCAAGCCAAAGATTTCGAATGGAGTTTTCCG
GGAGAGAAATGCAAGTTTCCTACGGCTCAAAACACGCCGAGATTCTCTTCATCAAT
GGCTAATAATAACTATTACTACACGCCCCCATCGCCGGCGAAAAGTGTTTGCAGA
GACGCTTGTTTTAGGCCAAGTTATCCTGGTTTGATGACACCTAGCTATATGGCTAA
TACGCAGTCGTTTAAAGCCAAGGTACGTTCGCATAGTGCACCGAGACAACGTCCT
GATAGAAAAAGATTGTCACTTGATGAGATTATGGCGGCTAGAAGTAGCGTTAGTG
GTGTGAGGATGGTGCAACCACAACCACAACCGCAAACGCAAACGCAGCAACAGA
AACGCTCTCCTTGTTCGTATGATCATCAGTTTCGTCAGAACGAGACTGATTTTAGA
TTCTATAATTAGTAAAAAACGTTATTTTCGTCCTTAAAGAAAATATCGTCATAGCCTT
TGACTTTTCATTTATGACTTTCCTTTTTTTTTTTTTTTTGTAAATTATTGCTTTGCTTT
GGAAAAAAT
SEQ ID NO: 74; CDS:
ATGGGAAGAGCTGCGAGATGGTTCAAGGGTATTTTTGGTATGAAGAAGAGCAAAG
AGAAAGAGAACTGTGTTTCCGGCGACGTTGGAGGTGAAGCCGGTGGTTCTAACAT
TCACCGGAAAGTTCTCCAAGCTGACTCCGTCTGGCTCAGAACTTACCTTGCGGAA
ACAGACAAAGAACAGAACAAACACGCGATTGCGGTTGCTGCTGCTACAGCCGCG
GCTGCTGACGCAGCGGTTGCAGCGGCTCAAGCTGCTGTGGCGGTGGTCAGGTTA
ACAAGTAACGGAAGAAGCGGAGGATATTCCGGGAACGCAATGGAGCGGTGGGCC
GCAGTGAAAATTCAATCAGTCTTCAAGGGCTATTTGGCGAGAAAAGCGTTACGAG
CTTTGAAAGGTTTAGTGAAGCTACAAGCTTTGGTAAGAGGATACTTAGTCCGCAAA
CGCGCCGCCGAAACGCTGCATAGCATGCAAGCTCTCATTAGAGCTCAAACCAGC
GTCCGATCGCAACGCATCAACCGCAACAACATGTTTCATCCTCGACACTCACTTG
AGAGGTTGGATGATTCAAGAAGTGAAATCCATAGCAAGAGAATATCAATCTCTGTA
GAGAAACAGAGTAATCACAACAACAATGCGTACGATGAGACCAGTCCCAAGATTG
TGGAGATTGATACTTACAAGACGAAATCAAGATCAAAGAGAATGAATGTGGCTGTA
TCCGAATGTGGAGATGATTTCATCTATCAAGCCAAAGATTTCGAATGGAGTTTTCC
GGGAGAGAAATGCAAGTTTCCTACGGCTCAAAACACGCCGAGATTCTCTTCATCA
ATGGCTAATAATAACTATTACTACACGCCCCCATCGCCGGCGAAAAGTGTTTGCA
GAGACGCTTGTTTTAGGCCAAGTTATCCTGGTTTGATGACACCTAGCTATATGGCT
AATACGCAGTCGTTTAAAGCCAAGGTACGTTCGCATAGTGCACCGAGACAACGTC
CTGATAGAAAAAGATTGTCACTTGATGAGATTATGGCGGCTAGAAGTAGCGTTAGT
GGTGTGAGGATGGTGCAACCACAACCACAACCGCAAACGCAAACGCAGCAACAG
AAACGCTCTCCTTGTTCGTATGATCATCAGTTTCGTCAGAACGAGACTGATTTTAG
ATTCTATAATTAG
SEQ ID NO: 75; Protein:
MGRAARWFKGIFGMKKSKEKENCVSGDVGGEAGGSNIHRKVLQADSVWLRTYLAET
DKEQNKHAIAVAAATAAAADAAVAAAQAAVAVVRLTSNGRSGGYSGNAMERWAAVKI
QSVFKGYLARKALRALKGLVKLQALVRGYLVRKRAAETLHSMQALIRAQTSVRSQRIN
RNNMFHPRHSLERLDDSRSEIHSKRISISVEKQSNHNNNAYDETSPKIVEIDTYKTKSR
SKRMNVAVSECGDDFIYQAKDFEWSFPGEKCKFPTAQNTPRFSSSMANNNYYYTPP
SPAKSVCRDACFRPSYPGLMTPSYMANTQSFKAKVRSHSAPRQRPDRKRLSLDEIM
AARSSVSGVRMVQPQPQPQTQTQQQKRSPCSYDHQFRQNETDFRFYN
GSE5-Like CRISPR sequences:
Rice:
Target sequence:
(SEQ ID NO: 76)
CGCCCCCGCCGGACAGGAAGCGG
Protospacer sequence:
(SEQ ID NO: 77)
CGCCCCCGCCGGACAGGAAG
Full sgRNA nucleic acid sequence; (SEQ ID NO: 78)
GTTTTAGAGCTAGAAATAGCAAGTTAAAATAAGGCTAGTCCGTTATCAACTTGAAA
AAGTGGCACCGAGTCGGTGC
CRISPR target sequences for “gse-5 like” in
Arabidopsis:
(SEQ ID NO: 79)
AAAGCGTTACGAGCTTTGAAAGG
Glycine max:
(SEQ ID NO: 80)
CTGACAAGAAGGAGAAGAAAAGG
Medicago truncatula:
(SEQ ID NO: 81)
TTTCACCTGCAGAAGCTGCTTGG
Sorghum bicolor:
(SEQ ID NO: 82)
GGCGACCGAGGGCTCCGTGCGGG
Triticum aestivum:
(SEQ ID NO: 83)
TCGTGCGGCTCACCAGCAAAGGG
Z.mays:
(SEQ ID NO: 84)
GACGGCATTCAGACGCTTCTTGG
Rice sgRNA sequence (SEQ ID NO: 89).
GTTTTAGAGCTAGAAATAGCAAGTTAAAATAAGGCTAGTCCGTTATCAACTTGAAA
AAGTGGCACCGAGTCGGTGCTTTTTTTGTCCCTTCGAAGGGCAATTCTGCAGATA
TCCATCACACTGGCGGCCGCTCGAGGTCGaagcttgcatgcctgcagg.

1. A method of increasing yield in a plant, the method comprising reducing or abolishing the expression of at least one (grain size on chromosome 5) GSE5 or GSE5-Like nucleic acid and/or reducing the activity of a GSE5 or GSE5-Like polypeptide in said plant.

2. (canceled)

3. (canceled)

4. The method of claim 1, wherein the method comprises introducing at least one mutation into the nucleic acid sequence encoding GSE5 or GSE5-Like or at least one mutation into the promoter of GSE5 or GSE5-Like.

5. The method of claim 4, wherein said mutation is a loss of function or partial loss of function mutation.

6. (canceled)

7. The method of claim 1, wherein the GSE5 nucleic acid encodes a polypeptide comprising SEQ ID NO: 1 or a functional variant or homolog thereof, and wherein the GSE5-Like nucleic acid encodes a polypeptide comprising SEQ ID NO: 57 or a functional variant or homolog thereof and wherein the GSE5 promoter comprises a nucleic acid sequence as defined in SEQ ID NO: 28 or a functional variant or homolog thereof.

8. (canceled)

9. (canceled)

10. (canceled)

11. (canceled)

12. The method of claim 1, the method comprising using RNA interference to reduce or abolish the expression of a GSE5 or GSE5-Like nucleic acid and/or reduce or abolish the activity of a GSE5 or GSE5-Like promoter.

13. (canceled)

14. The method of claim 1, wherein the plant is a crop plant and wherein the crop plant is selected from rice, wheat, maize, soybean and sorghum.

15. (canceled)

16. (canceled)

17. A genetically modified plant, plant cell or part thereof characterised by a reduced level of GSE5 or GSE5-Like nucleic acid expression and/or reduced activity of the GSE5 or GSE5-Like polypeptide.

18. (canceled)

19. (canceled)

20. (canceled)

21. The genetically modified plant of claim 17, wherein said plant comprises at least one mutation in at least one nucleic acid sequence encoding GSE5 or GSE5-Like or at least one mutation in the promoter of GSE5 or GSE5-Like.

22. The genetically modified plant of claim 21 wherein said mutation is a loss of function or partial loss of function mutation.

23. (canceled)

24. The genetically modified plant of claim 17, wherein the GSE5 nucleic acid encodes a polypeptide comprising SEQ ID NO: 1 or a functional variant or homolog thereof and wherein the GSE5-Like polypeptide encodes a polypeptide comprising SEQ ID NO: 57 or a functional variant or homolog thereof and wherein the GSE5 promoter comprises a nucleic acid sequence as defined in SEQ ID NO: 28 or a functional variant or homolog thereof.

25. (canceled)

26. (canceled)

27. (canceled)

28. (canceled)

29. The genetically modified plant of claim 17, wherein the plant comprises an RNA interference construct that reduces or abolishes the expression of a GSE5 or GSE5-Like nucleic acid and/or reduces or abolishes the activity of a GSE5 promoter.

30. The genetically modified plant of claim 17, wherein the plant is a crop plant and wherein the crop plant is selected from rice, wheat, maize, soybean and sorghum.

31. (canceled)

32. The genetically modified plant part of claim 17, wherein the plant part is a seed.

33. A method of producing a plant with increased yield, the method comprising introducing at least one mutation into at least one nucleic acid sequence encoding GSE5 or GSE5-Like and/or at least one mutation in the promoter of GSE5 or GSE5-Like.

34. (canceled)

35. (canceled)

36. (canceled)

37. (canceled)

38. (canceled)

39. The method of claim 33, wherein the plant is a crop plant and wherein the crop plant is selected from rice, wheat, maize, soybean and sorghum.

40. (canceled)

41. (canceled)

42. (canceled)

43. A method for identifying and/or selecting a plant that will have an increased seed yield phenotype, the method comprising detecting in the plant or plant germplasm at least one mutation in the promoter of the GSE5 gene, wherein said plant or progeny thereof is selected.

44. (canceled)

45. The method of claim 43 wherein said mutation is the deletion of a nucleic acid sequence comprising SEQ ID NO: 29 (DEL1) or SEQ ID NO: 30 (DEL2).

46. The method of claim 43, wherein said mutation is the insertion of a nucleic acid sequence comprising SEQ ID NO: 31 (IN1).

47. The method of claim 43, wherein the method further comprises introgressing the chromosomal region comprising at least one of said polymorphisms and/or deletions into a second plant or plant germplasm to produce an introgressed plant or plant germplasm.

48. A nucleic acid construct comprising a nucleic acid sequence encoding at least one DNA-binding domain that can bind to at least one GSE5 or GSE5-Like gene, wherein said sequence is selected from SEQ ID NOs: 15 to 20, 48, 51, 76 and 79 to 84 or a variant thereof.

49. (canceled)

50. (canceled)

51. (canceled)

52. (canceled)

53. (canceled)

54. (canceled)

55. The nucleic acid construct of claim 48, wherein the nucleic acid construct further comprises a nucleic acid sequence encoding a CRISPR enzyme.

56. (canceled)

57. (canceled)

58. The nucleic acid construct of claim 48, wherein the nucleic acid construct encodes a TAL effector and wherein the nucleic acid construct further comprises a sequence encoding an endonuclease or DNA-cleavage domain thereof.

59. (canceled)

60. (canceled)

61. (canceled)

62. An isolated plant cell transfected with at least one nucleic acid construct as defined in claim 48.

63. (canceled)

64. (canceled)

65. A genetically modified plant, wherein said plant comprises the transfected cell as defined in claim 62.

66. (canceled)

67. A nucleic acid construct comprising a nucleic acid sequence encoding a polypeptide as defined in SEQ ID NO: 1 or a functional variant or homolog thereof, wherein said sequence is operably linked to a regulatory sequence, wherein preferably said regulatory sequence is a tissue-specific promoter.

68. (canceled)

69. (canceled)

70. A transgenic plant expressing the nucleic acid construct of claim 69.

71. A method of increasing grain length, the method comprising introducing and expressing in said plant the nucleic acid construct of claim 67, wherein said increase is relative to a control or wild-type plant.

72. A method for producing a plant with increased grain length, the method comprising introducing and expressing in said plant the nucleic acid construct of claim 67, wherein said increase is relative to a control or wild-type plant.

73. (canceled)

74. (canceled)

75. (canceled)

76. (canceled)

77. (canceled)


SEQUENCE LISTING (127 sequences)

SEQ ID NO: 1 - protein, 470 aa, Oryza sativa
MGKAARWFRNMWGGGRKEQKGEAPASGGKRWSFGKSSRDSAEAAAAAAAAAAEASGGNAAIARAAEAAWLRSVYADTEREQSKHAIAVAAATAAAADAAVAAAQAAVAVVRLTSKGRSAPVLAATVAGDTRSLAAAAVRIQTAFRGFLAKKALRALKALVKLQALVRGYLVRRQAAATLQSMQALVRAQATVRAHRSGAGAAANLPHLHHAPFWPRRSLQERCAGDDTRSEHGVAAYSRRLSASIESSSYGYDRSPKIVEVDTGRPKSRSSSSRRASSPLLLDAAGCASGGEDWCANSMSSPLPCYLPGGAPPPRIAVPTSRHFPDYDWCALEKARPATAQSTPRYAHAPPTPTKSVCGGGGGGGIHSSPLNCPNYMSNTQSFEAKVRSQSAPKQRPETGGAGAGGGRKRVPLSEVVVVESRASLSGVGMQRSCNRVQEAFNFKTAVVGRLDRSSESGENDRHAFLQRRW

SEQ ID NO: 2 - DNA, 1413 bp, Oryza sativa
atgggcaaggcggcgcggtggttccgcaacatgtggggaggagggaggaaggagcagaagggcgaggcgccggcgagtggggggaagaggtggagcttcgggaagtcgtcgagggactcggcggaggccgcggcggctgctgctgcggcggcggcggaggcttccgggggcaatgcggcgatcgccagggcggccgaggcggcgtggctcaggtcggtgtacgccgacacggagcgggagcagagcaagcacgccatcgccgtcgccgcggccaccgcggcggcggctgatgccgccgtggcggccgctcaggccgccgtcgccgtcgtgcggcttactagcaagggccgctcggctcccgtcctcgccgccaccgtcgccggcgacacgcgcagccttgccgccgccgccgtcagaatccagacggcattcagaggcttcctggcgaagaaggcgctgcgagcgctcaaggcgctggtgaagctgcaggcgctggtgcgcggctacctcgttcgccggcaggccgccgccacgctgcagagcatgcaggcgctcgtccgcgcccaggccactgtccgcgcccaccgcagtggcgccggcgccgccgccaatctcccgcacctccaccacgctcccttctggccccgccgctcgctgcaggagaggtgcgccggcgacgacacgaggagcgagcacggtgtggcggcgtacagccggcggctgtcggcgagcatcgagtcgtcgtcgtacgggtacgaccggagccccaagatcgtggaggtggacaccgggaggcccaagtcgcggtcgtcgtcgtcgcggcgggcgagctccccgctgctgctcgacgccgctgggtgcgcgagcggcggcgaggactggtgcgccaactccatgtcgtcgccgctcccgtgctacctccccggcggcgcgccgccgccccgcatcgccgtcccgacgtcgcgccacttccccgactacgactggtgcgcgctggagaaggcccggccggcgacggcgcagagcacgccgcggtacgcgcacgcgccgccgacgccgaccaagagcgtgtgcggcggcggcggcggcggcggcatccactcgtcgccgctcaactgcccgaactacatgtccaacacgcagtcgttcgaggcgaaggtgcgttcgcagagcgcgccgaagcagcggccggagaccggcggcgccggcgccggcggcggccggaagcgggtgccgctgagcgaggtggtggtggtggagtccagggcgagcttgagcggcgtgggcatgcagcgctcgtgcaaccgggtgcaggaggcgttcaacttcaagacggccgtcgtcggccgcctcgaccgctcgtcggagtccggcgagaacgaccgccacgcgttcttgcagaggaggtggtga

SEQ ID NO: 3 - protein, 463 aa, Triticum aestivum
MGKAARWLRGLLGGGGKKEQGKEQRRPATAPHGDRKRWSFCKSTRDSAEAEAAAAAAALSGNAAIARAAEAAWLKSLYNETEREQSKHAIAVAAATAAAADAAMAAAQAAVEVVRLTSKGPTSTVLADAVAEPHGRASAAVKIQTAFRGFLAKKALRALKGLVKLQALVRGYLVRKQAAATLQSMQALVRAQACIRAARSRAAALPTNLRVHPTPVRPRYSLQERYSTTEDSRSDHRVAPYYSRRLSASVESSSCYGYDRSPKIVEMDTGRPKSRSSSLRTTSPGASEECYAHSVSSPLMPCRAPPRIAAPTARHFPEYEWCEKARPATAQSTPRYTSYAPVTPTKSVCGGYTYSNSPSTLNCPSYMSSTQSSVAKVRSQSAPKQRPEEGAVRKRVPLSEVIILQEARASLGGGGGTQRSCNRPAQEEAFSFKKAVVSRFDRSSEAAERERDRDRDLFLQKGW

SEQ ID NO: 4 - DNA, 1392 bp, Triticum aestivum
atgggcaaggcggcgaggtggctgcgtggcttgctgggcggcggcggcaagaaggagcaggggaaggagcagaggcgcccggccacggcgccgcacggggacaggaagcgctggagcttctgcaagtccaccagggactcggcagaggcggaggcggcggccgcggccgcggcgctcagcggcaacgcggcgatcgcgcgcgcggccgaggcggcatggctcaagtccttgtacaacgagaccgagcgcgagcagagcaagcacgccatcgccgtcgccgcggccaccgcggcggcggcggacgcggctatggctgccgcacaggcagccgtggaggtcgtgcggctcaccagcaaagggccgacgtcgacggtgctcgccgacgccgtcgcggagccccacggccgtgcctccgccgcggtcaagatccagacggcgttccgtggcttcctggccaagaaggctctgcgcgcgctcaaggggctggtgaagctgcaggcgctggtgcgcggctacctggtgcggaagcaggcggcggccacgctgcagagcatgcaggcgctcgtccgcgcgcaggcctgcatccgcgctgcccgctcgcgcgccgcggcgctcccgacgaaccttcgcgtccaccccactcctgtccggccgcgctactcgttgcaagagcggtacagcaccacggaggattcccggagcgaccaccgcgtggcgccgtactacagccgccggctgtcggcgagcgtggagtcgtcgtcgtgctacggctacgaccggagccccaagatcgtggagatggacaccggccggcccaagtcgcgctcctcctcgctccggacgacctcccccggcgccagcgaggagtgctacgcccactcggtgtcgtcgccgctcatgccgtgccgagcgcccccgcggatcgcggcgcccaccgcgcgccacttcccggagtacgagtggtgcgagaaggcccggccggcgacggcgcagagcacgccccggtacacgagctacgcgccggtgacgccgaccaagagcgtgtgcggcggctacacctacagcaacagcccgtcgacgctcaactgccccagctacatgtcgagcacgcagtcgtccgtggcgaaggtgcgttcgcagagcgcgccgaagcagcggccggaggagggcgcggtacggaagagggtgccgctgagcgaggtgatcatcctgcaggaggcccgggcgagcctgggcggcggcgggggcacgcagaggtcgtgcaaccggccggcgcaggaggaggcgttcagcttcaagaaggccgtcgtgagccgcttcgaccgctcgtcggaggcggccgagagggaacgtgaccgggaccgggacttgttcctgcagaaggggtggtga

SEQ ID NO: 5 - protein, 483 aa, Zea mays
MGKAARWFRSFLGKKEQRPTKDQRRLQQQDDQAPPLPPPSAKRWSFGRSSRDSAAAAVVSAGAGNAAIARAAEAAWLRSAACAETHRDRDQDQDQSKHAIAVAAATAAAADAAVAAAQAAVAVVRLTSKGRAPLFAVAAAVRIQTAFRGFLCSVAALPRCVPSTQAKKALRALKALVKLQALVRGYLVRRQAAATLQSMQALVRAQATVRARRAGAAALPHLHHLPGRPRYSMQERCADDARIEHGVAAHSSRRLSASVESSSYGYDRSPKIVEVDPGRPKSRSSSRRSSAPLLDAGSCCGEEWCASANPASSPLPCYLSAGPPTRIAVPTSRQFPDYDWCALEKARPATAQSTPRCLLQAHAPATPTKSVVAGHSPSLNGCPNYMSSTQASEAKARSQSAPKQRPELACCCGGARKRVPLSEVVLVDSSRASLSGVVGMQRGCSTGAQEAFSFRTAVVGRIDRSLEVAGGENDRLALLQRRW

SEQ ID NO: 6 - DNA, 1452 bp, Zea mays
atgggcaaggcggcgcgctggttccgcagcttcctgggcaagaaagagcagcggcccaccaaggaccagcggcggctgcagcagcaggacgaccaggctcctccgcttccgccgccaagcgccaagcgctggagcttcggtaggtcgtcgcgggactcggcggcggccgcggtcgtctcggccggcgcgggcaacgcggcgatcgcgcgcgccgccgaggccgcgtggctcaggtccgccgcgtgcgccgagacgcaccgggaccgggaccaggaccaggaccagagcaagcacgccatcgccgtggccgccgccaccgccgccgcggcggacgcggcggtggcggcggcgcaggcggcagtcgccgttgtgcgcctcaccagcaagggacgcgcgccgctcttcgccgtcgccgccgccgtcaggatccagacggcgttccgaggattcttgtgttctgttgctgccctgccgcggtgtgttccttctacgcaggccaagaaggcgttgcgcgcgctcaaggcgctcgtgaagctgcaggcgctggtgcgcggctacctcgtgcgcaggcaggcggccgccacgctgcagagcatgcaggctctcgtccgcgcgcaggccaccgtgcgcgcgagacgagccggcgccgccgccctcccgcacctccaccacctgcccggccgcccgcgctactcgatgcaagagcggtgcgcggacgacgcgcggatcgagcacggggtggcggcgcacagcagccggcggctgtcggcgagcgtggagtcctcgtcgtacggctacgaccggagtcccaagatcgtggaggtggaccccggccgccccaagtcgcggtcgtcctcgcgccgctcgagcgccccgctgctcgacgccggcagctgctgcggcgaggagtggtgcgccagcgccaaccccgcgtcctcgccgctgccgtgctacctgtccgccgggccgccgacgcgcatcgccgtgccgacctcgcgccagttcccggactacgactggtgcgcgctggagaaggcccggccggccacggcgcagagcacgccgcggtgcctgctgcaggcgcacgcgccggccaccccgaccaagtccgtcgtcgcgggccactcgccgtcgcttaacgggtgcccgaactacatgtcgagcacgcaggcgtcggaggccaaggcgcggtctcagagcgcgccgaagcagcggcccgagctcgcctgctgctgcggcggagcgcgcaagcgggtgccgctcagcgaggtggttctcgtggattcctcccgcgccagcctgagcggcgtcgtgggcatgcagcgcgggtgcagcaccggggcgcaggaggcgttcagcttccggacggccgtcgttggtcgcatagaccgctcgttggaggttgccggcggcgagaacgaccggctggccttgttgcagaggaggtggtga

SEQ ID NO: 7 - protein, 420 aa, Glycine max
MGRATRWVKSLFGIRREKEKKLNFRCGEAKSMELCCSESTSNSTVLCHNSGTIPPNLSQAEAAWLQSFCTEKEQNKHAIAVAAATAAAADAAVAAAQAAVAVVRLTSQGRGRTMFGVGPEMWAAIKIQTVFRGFLARKALRALKGLVKLQALVRGYLVRKLATATLHSMQALVRAQARMRSHKSLRPMTTKNEAYKPHNRARRSMERFDDTKSECAVPIHSRRVSSSFDATINNSVDGSPKIVEVDTFRPKSRSRRAISDFGDEPSLEALSSPLPVPYRTPTRLSIPDQRNIQDSEWGLTGEECRFSTAHSTPRFTNSCTCGSVAPLTPKSVCTDNYLFLRQYGNFPNYMTSTQSFKAKLRSHSAPKQRPEPGPRKRISLNEMMESRNSLSGVRMQRSCSQVQEVINFKNVVMGKLQKST

SEQ ID NO: 8 - DNA, 1263 bp, Glycine max
atggggagagccactaggtgggtgaagagtttgtttggaataagaagagagaaagagaagaaactaaacttcaggtgtggagaggctaaaagtatggaattgtgttgttctgagagtactagtaattcaacagttttgtgtcacaattcagggactataccccccaacctttctcaagctgaggctgcttggttacaatcattctgcacagagaaggagcaaaacaagcacgccatcgcagttgctgctgccacggcggcagctgctgatgctgccgtggcagcagcacaggctgcggtggcggttgttaggctcaccagccaaggaaggggtcgcaccatgtttggtgttggacctgagatgtgggctgccatcaagattcaaacagtgtttagaggattcctggcaaggaaggcactaagggcattaaaaggattggtgaaattgcaggcacttgtcagagggtatttagtgaggaagctagcaacagcaaccctgcatagtatgcaggctcttgttagagctcaagctagaatgcggtcccacaaatctctcaggcccatgaccacaaagaatgaagcatataaacctcataatagagcaagaagatccatggagaggtttgatgacactaagagtgagtgtgcagttccaatccacagtagaagggtatcatcttcttttgatgctacaattaacaacagtgttgatgggagccccaaaatagtggaagtggacactttcaggcctaagtcaaggtctagaagggcaatttcagattttggtgatgaaccatcactagaagcactttcttctcccttaccagttccgtacagaacccctacacgtttgtccataccagaccaaaggaatattcaggactctgaatgggggttaacaggagaagagtgcagattctctacagcacatagcactccgcgcttcacaaattcttgtacctgtggctcagttgcaccattgacaccaaagagtgtgtgcactgataactacttgttcctaaggcagtatgggaattttccaaactacatgactagtactcagtcttttaaggccaaattgaggtctcatagtgctccaaagcaacggccagaacctggtccaaggaagaggatttccctcaatgaaatgatggagtctaggaatagtttgagtggggttagaatgcagaggtcttgctcacaggttcaagaagtcattaatttcaagaatgttgtgatggggaagcttcagaaatccacataa

SEQ ID NO: 9 - protein, 493 aa, Sorghum bicolor
MGKAARWFRSFLGGKKEQQATKDHRRRQQQQQQDQPPPPPPPPATTAKRWSFGKSSRDSAEAAAAVVSAGAGNAAIARAAEAAWLRSAACAETDREREQSKHAIAVAAATAAAADAAVAAAQAAVAVVRLTNKGRAPPGVLATAGGGRAAAAAVRIQTAFRGFLAKKALRALKALVKLQALVRGYLVRRQAAATLQSMQALVRAQAAVRARRAAAAALSQSHLHHHHHPPPVRPRYSLQERYADDTRSEHGVAAYSSRRLSASVESSSYGGYDRSPKIVEVDPGRPKSRSSSSRRASSPLLDAAGGSSGGEDWCAANPASSSPLPCYLSAAGGPPRIAVPTSRQFPDYDWCALEKARPATAQSTPRYLLPATPTKSVAGNSPSLHGCPNYMSSTQASEAKVRSQSAPKQRPELACCAGGGGGGARKRVPLSEVVVVESSRASLSGVVGMQRGCGGARAQEAFSFRAAVVGRMDRSLEVAGIENDRQAFLQRRW

SEQ ID NO: 10 - DNA, 1482 bp, Sorghum bicolor
atgggcaaggcggcgcgctggttccgcagcttcctgggcggcaagaaggagcagcaggccaccaaagatcaccggcggcgccagcagcagcagcagcaggaccagcctcctcctcctccgcctccgccggccaccaccgccaagcgctggagcttcggcaagtcgtcgcgggactcggccgaggcggccgcggccgtcgtctcggccggcgcgggcaacgcggcgatcgcgcgcgccgcggaggccgcctggctcaggtccgccgcgtgcgccgagacggaccgcgagcgggagcagagcaagcacgccatcgccgtggccgccgccaccgccgccgcggccgacgcggcggtcgccgcggcgcaggcggccgtcgccgtcgtccgactcacaaacaagggacgcgcgccgcccggcgtcctcgccaccgctggaggaggacgcgccgccgccgccgccgtcaggatccagacggcgttccgaggattcttggcgaagaaggcgttgcgcgcgctcaaggcgctcgtgaagctgcaggcgctggtgcgcggctacctcgtgcgcaggcaggcggccgccacgctgcagagcatgcaggcgctcgtccgcgcgcaggccgccgtgcgcgccaggcgcgccgccgccgccgcgctctcgcagtcgcacctccaccaccaccaccacccgccgcccgtccgtccgcgctactcgctgcaagagcggtacgcggacgacacgcggagcgagcacggggtggcggcgtacagcagccggcggctgtcggcgagcgtcgagtcctcgtcgtacggcggctacgaccggagccccaagatcgtggaggtggacccgggccgccccaagtcgcgctcgtcgtcctcgcgccgggccagctcaccgctgctcgacgccgccggcggcagcagcggcggcgaggactggtgcgccgccaaccccgcgtcgtcgtcgccgctgccgtgctacctgtccgccgccggcggaccgccgcgcatcgccgtgccgacctcgcgccagttcccggactacgactggtgcgcgctcgagaaggcccgcccggccacggcgcagagcacgccgcggtacctgctgccggccaccccgacgaagtccgtcgcgggaaactcgccgtcgctgcacgggtgcccgaactacatgtcgagcacgcaggcgtcggaggccaaggtgcggtcccagagcgcgcccaagcagcggcccgagctcgcctgctgcgccggcggcggcggcgggggagcgcggaagcgggtgccgctcagcgaggtggtggtcgtggagtcgtcccgcgccagcctgagcggcgtcgtgggcatgcagcgcgggtgcggcggcgcccgggcgcaggaggcgttcagcttcagggcagccgtcgttggccgcatggaccgctcgttggaggttgccggtatcgagaacgaccggcaggcgttcttgcagaggaggtggtga

SEQ ID NO: 11 - protein, 429 aa, Medicago truncatula
MGRTIRWFKSLFGIKKDRDNSNSNSSSTKWNPSLPHPPSQDFSKRDSRGLCHNPATIPPNISPAEAAWVQSFYSETEKEQNKHAIAVAALPWAVVRLTSHGRDTMFGGGHQKFAAVKIQTTFRGYLARKALRALKGLVKLQALVRGYLVRKQATATLHSMQALIRAQATVRSHKSRGLIISTKNETNNRFQTQARRSTERYNHNESNRNEYTASIPIHSRRLSSSFDATMNSYDIGSPKIVEVDTGRPKSRSRRSNTSISDFGDDPSFQTLSSPLQVTPSQLYIPNQRNYNESDWGITGEECRFSTAQSTPRFTSSCSCGFVAPSTPKTICGDSFYIGDYGNYPNYMANTQSFKAKLRSHSAPKQRPEPGPKKRLSLNELMESRNSLSGVRMQRSCSQIQDAINFKNAVMSKLDKSTDFDRNFSKQRRL

SEQ ID NO: 12 - DNA, 1290 bp, Medicago truncatula
atgggtagaaccataaggtggttcaagagtttgtttgggataaagaaagacagagataattcaaactcaaattcttcaagtaccaaatggaatccttctcttcctcatcctccttctcaagatttctcaaagagagattcgagaggcttgtgtcataatccagctaccatacctcccaacatttcacctgcagaagctgcttgggttcaatccttctactcagaaactgagaaggagcaaaacaagcacgccattgcggtagcagctctgccgtgggctgtggttagattaaccagccacggcagagacaccatgtttggtggtggacaccagaaatttgctgctgtcaagattcaaacaacatttaggggttacttggcaagaaaagcactaagagccttaaagggattggtaaagttacaagcactagtgagagggtacttagtgaggaagcaagcaacagcaacattacacagtatgcaagctctaattagagcacaagcaacagtaaggtctcataaatctcgtggactcatcataagcacaaagaatgaaacaaataacagatttcaaacacaagctagaagatccacggaaaggtataatcacaatgagagtaacaggaacgagtacacagcttcaattcctattcacagcagaagattatcatcatcttttgatgctacaatgaacagttatgatattggaagtccaaaaatagtagaagttgatactggaagaccaaaatcaaggtctagaagaagcaatacatcaatttcagattttggagatgacccttcatttcaaacactttcttctccacttcaagttactccatctcagttatacattccaaatcaaagaaattataacgaatcagattggggaataacaggtgaagaatgcagattttcaactgcacagagcactccacgtttcacaagttcatgtagttgtggatttgttgcaccttccacacctaaaacaatttgtggagatagtttttacattggtgattatggtaattatcctaattacatggctaatacacagtcttttaaggctaaattgaggtctcatagtgctccaaagcaacgacctgaaccaggtccgaagaagaggctttcattgaatgaattgatggaatctagaaacagtttgagtggagttagaatgcagaggtcttgttcacagattcaggatgctattaattttaagaatgctgtgatgagtaaacttgataagtccactgattttgatagaaacttttcaaagcagaggaggttgtga

SEQ ID NO: 13 - protein, 389 aa, Arabidopsis thaliana
MGRAARWFKGIFGMKKSKEKENCVSGDVGGEAGGSNIHRKVLQADSVWLRTYLAETDKEQNKHAIAVAAATAAAADAAVAAAQAAVAVVRLTSNGRSGGYSGNAMERWAAVKIQSVFKGYLARKALRALKGLVKLQALVRGYLVRKRAAETLHSMQALIRAQTSVRSQRINRNNMFHPRHSLERLDDSRSEIHSKRISISVEKQSNHNNNAYDETSPKIVEIDTYKTKSRSKRMNVAVSECGDDFIYQAKDFEWSFPGEKCKFPTAQNTPRFSSSMANNNYYYTPPSPAKSVCRDACFRPSYPGLMTPSYMANTQSFKAKVRSHSAPRQRPDRKRLSLDEIMAARSSVSGVRMVQPQPQPQTQTQQQKRSPCSYDHQFRQNETDFRFYN

SEQ ID NO: 14 - DNA, 1170 bp, Arabidopsis thaliana
atgggaagagctgcgagatggttcaagggtatttttggtatgaagaagagcaaagagaaagagaactgtgtttccggcgacgttggaggtgaagccggtggttctaacattcaccggaaagttctccaagctgactccgtctggctcagaacttaccttgcggaaacagacaaagaacagaacaaacacgcgattgcggttgctgctgctacagccgcggctgctgacgcagcggttgcagcggctcaagctgctgtggcggtggtcaggttaacaagtaacggaagaagcggaggatattccgggaacgcaatggagcggtgggccgcagtgaaaattcaatcagtcttcaagggctatttggcgagaaaagcgttacgagctttgaaaggtttagtgaagctacaagctttggtaagaggatacttagtccgcaaacgcgccgccgaaacgctgcatagcatgcaagctctcattagagctcaaaccagcgtccgatcgcaacgcatcaaccgcaacaacatgtttcatcctcgacactcacttgagaggttggatgattcaagaagtgaaatccatagcaagagaatatcaatctctgtagagaaacagagtaatcacaacaacaatgcgtacgatgagaccagtcccaagattgtggagattgatacttacaagacgaaatcaagatcaaagagaatgaatgtggctgtatccgaatgtggagatgatttcatctatcaagccaaagatttcgaatggagttttccgggagagaaatgcaagtttcctacggctcaaaacacgccgagattctcttcatcaatggctaataataactattactacacgcccccatcgccggcgaaaagtgtttgcagagacgcttgttttaggccaagttatcctggtttgatgacacctagctatatggctaatacgcagtcgtttaaagccaaggtacgttcgcatagtgcaccgagacaacgtcctgatagaaaaagattgtcacttgatgagattatggcggctagaagtagcgttagtggtgtgaggatggtgcaaccacaaccacaaccgcaaacgcaaacgcagcaacagaaacgctctccttgttcgtatgatcatcagtttcgtcagaacgagactgattttagattctataattag

SEQ ID NO: 15 - DNA, 23 bp, Artificial Sequence
  Oryza sativa target sequence
cgaggcggcgtggctcaggtcgg

SEQ ID NO: 16 - DNA, 23 bp, Artificial Sequence
  Triticum aestivum target sequence
cagcaaagggccgacgtcgacgg

SEQ ID NO: 17 - DNA, 23 bp, Artificial Sequence
  Zea mays target sequence
ccgcgtgcgccgagacgcaccgg

SEQ ID NO: 18 - DNA, 23 bp, Artificial Sequence
  Glycine max target sequence
aggctgcggtggcggttgttagg

SEQ ID NO: 19 - DNA, 23 bp, Artificial Sequence
  Medicago truncatula target sequence
ttctcaaagagagattcgagagg

SEQ ID NO: 20 - DNA, 23 bp, Artificial Sequence
  Arabidopsis thaliana target sequence
acagaacaaacacgcgattgcgg

SEQ ID NO: 21 - DNA, 20 bp, Artificial Sequence
  Oryza sativa protospacer sequence
cgaggcggcgtggctcaggt

SEQ ID NO: 22 - DNA, 20 bp, Artificial Sequence
  Triticum aestivum protospacer sequence
cagcaaagggccgacgtcga

SEQ ID NO: 23 - DNA, 20 bp, Artificial Sequence
  Zea mays protospacer sequence
ccgcgtgcgccgagacgcac

SEQ ID NO: 24 - DNA, 20 bp, Artificial Sequence
  Glycine max protospacer sequence
aggctgcggtggcggttgtt

SEQ ID NO: 25 - DNA, 20 bp, Artificial Sequence
  Medicago truncatula protospacer sequence
ttctcaaagagagattcgag

SEQ ID NO: 26 - DNA, 20 bp, Artificial Sequence
  Sorghum bicolor protospacer sequence
gtcgagtcctcgtcgtacgg

SEQ ID NO: 27 - DNA, 76 bp, Artificial Sequence
  CRISPR sequence
gttttagagctagaaatagcaagttaaaataaggctagtccgttatcaacttgaaaaagtggcaccgagtcggtgc

SEQ ID NO: 28 - DNA, 6320 bp, Oryza sativa
atgttataccgccgttctgcctgttattttggtgtatggtgaatgttatggagtattgcttcatttttaaatgaaaggcaccatcttagattttttttcctttagttgattggttgtagtactatattttctaagttattataaaataaatatgcgatatatatttgattgccattatcgagttagtatatcttcactctgtttatattttaaagctgtgtactcctagctatagcactaaagaccgaacttagtatggactaattacagcgataaccatcggtaattgctactactccatgcagtacttggtttccatatttttgcatgcgtcggtcgttggaggcaagcaagcaagctgcagcgtcgtcagaggtagacgcatcgatgttgttcttccatgtccttcgaatcttcatgattgccgctctctgcttaattactgttctcttcacgcacaccgtccaaatcccatccatctatccatccgtccacacacctgcaagtttaaattcccatgcagttactacatgaacagtactgctgtcaagtttaattaatttctactacacacgaattgaattcaaatggtactaggtacaagtactccactagtacgaccatgatgtttcccacctgaaagcaacacagaatacgttcgtacgtacacgtacagtaaaccacaataatgcacaagagtagggatcgatggagagtacatgcaattgctcatcacttgtcaccatcactggttcaaatttgcctactctagatctcgatcgtggtactgcttctaaccataatgatcggtacatgttcaaattcgaaactcaaaaaatgtgtgtacaatacggccgggagagatgaacatatatacagtactcataattattaaccatgcatgggcattaataaaagtagtactccgtatatagcttccaagcaaggctacatactagaatcatactgtatcttgattgccagaggtgcaatcatgcatagacatacttaagatcagggctagctagttcaggtgatgaacacatgctttgtaccccttgtaggatgcatggcaccaagtcgacattggagtacacacaccctggccctgctgcacgtgtagataagagtaccacctatcatcatcaatcacgcaatatggtctactgtgtctctttgattgggtgtaatataatatcggccactgtaaaaatttaaaatttaaaacttagtaattttaatttcgaagttgattttatgatatgctcaacgtatctcttttttttcaagctagcgttggtctttaaaagtgcgtttgtttataaggttagggtgagacttttagcttgtgtttggttagtgggatatggaatgagatgggttaggtcgatcctatttttcgagctgtttgggtagagggatgtgtaggacgggatgatcctagatgggaatattcttcccagatccaggacgaggtggtccgccaaaatcggctggactaatccatcccactttgatggagcgaatggcattagctccttccgcctcctgctcggctccgccactggccgcccgttcctcctcctccgtctgctccctcccacggcgtccgtccctcctcctcttcctgctccctcccccgatgccgctccgtcctcctccgcctgctccctcccccggcgcccgtccctcctcctctgcccgctcgccaagttgccggctgcaccgcccgctcgtgcagctcctcctgccatccgcgccgccggtcgctgatcgcgccgctcctccttctgcctactccctccccggcaccgctcctccttctgcctactccctcccccggcaccactccgtcctcctccgcttgctccctcccccggcgccgctccctcccccagcgcccgttagtcctcctccgcccgctcatgcagctccttccctacacacgctcgctcgcccgcgccgctcctcattctgccgttcgccgctcacgcctgtgcctgctcgcttgcacgccgctccttccatccacaccgccggtcgccggttgtttgccgctcctgcttccaccgcccgccgttcgctgctccttcctcactaggagtagacgtggtgctaacatctccaaacgtcattcatcccatccccacccctatccattctaccaaacaaaaaactagcatcatctcattttataaaccaaacaagtatgtgagatcacccaatccctaaaatcagggatggtttcatcctatcccacatagtccccaaacaaaacacatgcttagttgtacgcaaaacgagaaagcttatgagcacatggtttataactatcataaactcaaaattttggtttttttgaaagaaactaattatatgtagaaaagtttttttaaaaaaaataaacatagtttaacagtttagaaaacgtactaacagaaaatgagaaagttaccgctcagatcttagacttaactccatagactcatatgaaaggtcactcataaattatttttcacgaataagctgtttggtatataaatcagctctcggtcaaccgatgaggttgtactttggatacatgtgtgcgagtgcatctttcgtgtataacgtggccctgttcttcccctccccacacacatgaatgtgtgtgtatttaaacggcttttggggggtcacctttcgcaggtactataccccaagagctgaaaaaattgcaaggccggggcttagccatatctgctagcagaaacctgtaggctggatcatgtaccagctgcatttgatgcataccctatgctttagctaaggaggagtacgatcgattcatcaatatcgctcgatcggtgacgacgtcgtccctgcgacatcagatcgtactactgctacagtacagcttttgcctgttccaatcactaaccagcctgcctctctctctctctctgccatggatggctagctagctagatcgatctatcgatgcagagtgattagctagctagctagaattggtgaattgtggtgacgacgagatcgttagcaacagtggccacgagtcaagatgctgtatatatgtatggatgagtcatcagtgtgatgcatgatctcacatctcgtcactgatgatctccagcttgagcttgcgctgaggtgagctgagctacactgctgcttctttgaccttctccatcgatcgaatcccgtggtgataaactgttaactgaggtaattgtaactactgcagcttcgtctctctctctctctctctcacgaaagatgcgtgatgctgatgcatatgcggttttttggatgtactgtttggacggttgcatactgtgcccgttcaatgtcagcatctccatcttcatatgttgctgccccgcctcatctcgatcgccaacttctattgtttcgcttgtgcttatacttataagttaaaatttaaattttaaaatttagttttgaagttaattttaagattttttttatcatagtttatttcacaccattatttgcttttcagtagtttaataacatataaataaaagttatatttataggttagttttgagagcactgctcccgtccagcaaacggtacccccaggtaccggtacccctggtacgaaacttaatttgaccattgaattagagcggggcacgatcgggattgaacttaatctcgcgaggactcagtctcctgcccatgcgctgcatccgcatctgaagtctacgccgtgtcaccgccgcaatcctttcgccctactccgacgcggtgtccaagtgccgtcctcttccggcctctgatgcggtggtgtggcggctcgcctctcggctgcccctgtgcggtacggcagctcgtccttcgtccacggtcacaacttgtcccctcccctccatcctctatgcatctcggctggtggcacctacgcagtctccggcacaggatcacaacaccctgagtttctttacggaagatgtatgagagagatgaagtatttctttcctgctggacattgctttgctgctatattcattggaagaatctgctatgttgatgggagaggctgagtttgatttatgttgtgtgccaagttctggacctagttccttgtcttttgatatatgtaatgaacctacttgattttgctgaaagtatgaatgtaatagtagtaaaaaagtagatgttctgaaagtttgtagtttcttgctctgatgtgtaaactgttctttcgttgtagaccttaagcttactgtttcatcttaaacaaaattaacatcggcggtcattgagcaaattgtcaactatcttgaataaaagggacactattacaactagtgatcccataaataaacttctgaaattcttcgatctcttttctttgcttgcccaatttcttcttgcttgtgcgatccatggccaaaagcctttcagccatctcaatatctagcttcgttttttcttctttctgtcccctccttctaggtttgctaatgctcgcgatccaatgcataccacagcgatgcgattccctgaacaaaggcttgcgatggcgtcggtagacgagctgtcgaggccatattcgccgcgctgagctagtcgccatggtcacctcttcttgttggtgcattgaggtcatcatcatccaggggtagatcgactcccagaaatcggcggcggccggacctggaccggctacgggaagggcaagttgggaatgggcaggttggcgacggttggttgcagtgagggaggaagcgaagtccggcgagattggctgtgccgccgccagggacgacggaaggggatcgggaagagacagattaccgggtcagggacttgtaccgtctctatgaaaattgactaaccacgtaaaaatccaattctgcccttccactagttccatacacgcgaacaaagggggaggtaccctgcaccggattcctgtacctcgcggtaccgatgattgtgggtcgtttgatctggctgaatggacggttacgattgcagtaccgcgtggtaccaaaaattctgctggacagtaaaaaatctctttgatttgctaataagcagtatgaataaacatatgcataagcgaaaggctaggtgctactacttctactgtggaagtgctcatggaggggggccgaatggccgatcgatcgagaaagcattcatccattccattctctcgctctctcaaaagttgcaatcttttttttttctcctcttcttctttttctttttcgttttccagctcatctctgatggatcatctctctctccccgtgtggtaattccgatgtgatcgatgacgcgtgcatgcgtcggagtaggagtacagcctctgttgttctttttggtcttcttcttctacttcctcccaaaaatgcgttgtgagcgagaaaaagagaagcttttttttggtgtgcgtgagtgtgcaactctcaatatttgttgcccgaaatctttcgagtttgcgtctttttgggcttacactgtcccttttttatcgctgcgtccaatcctaatccactatttatgcctaattaattactccgtctgttcttgaatatgacaacttaacttaagtagtggattgaaccttacgtggtactgtactatatatctagacatacattatatctagatatattgtaccaggtaatatctcatatagtactaggatgttatattctctggtactctctaccggtagtacagtaggaatggtctgaattgttcgttattaagggtaaattataaatttactaatataatgattacggaatactttattatttatgttttttttgtctaaatcaatttatggtcatccattttattggcatcactcatattttatcgaaaagaaaaataaaagagaaagaagatttagtcactaataaatgacgattataccttacacctatttttttacaataatgctccctcaaaatttctataaccactaactgaaatggtaatacaaatagatctaaaccactagataaaaaagataaactgttcatattgctttctatcagtagcctgggattgggacggaggaagcgaagagagagaaagagagaggtggttttgttttgtttgtccggtaatggctgccgcattggtggtggtggcctcctctcctcttcttttatttcgaacgcgacgccacccacgcgcctccccctcccccctgcggtttccctctcttattcaaaacctgtctcgattctcactcactctcactcactcggactcctcacccgctagctaccccggagcgcgccgcgccaccgctcgacagcggcgag

SEQ ID NO: 29 - DNA, 950 bp, Oryza sativa
tcatttttaaatgaaaggcaccatcttagattttttttcctttagttgattggttgtagtactatattttctaagttattataaaataaatatgcgatatatatttgattgccattatcgagttagtatatcttcactctgtttatattttaaagctgtgtactcctagctatagcactaaagaccgaacttagtatggactaattacagcgataaccatcggtaattgctactactccatgcagtacttggtttccatatttttgcatgcgtcggtcgttggaggcaagcaagcaagctgcagcgtcgtcagaggtagacgcatcgatgttgttcttccatgtccttcgaatcttcatgattgccgctctctgcttaattactgttctcttcacgcacaccgtccaaatcccatccatctatccatccgtccacacacctgcaagtttaaattcccatgcagttactacatgaacagtactgctgtcaagtttaattaatttctactacacacgaattgaattcaaatggtactaggtacaagtactccactagtacgaccatgatgtttcccacctgaaagcaacacagaatacgttcgtacgtacacgtacagtaaaccacaataatgcacaagagtagggatcgatggagagtacatgcaattgctcatcacttgtcaccatcactggttcaaatttgcctactctagatctcgatcgtggtactgcttctaaccataatgatcggtacatgttcaaattcgaaactcaaaaaatgtgtgtacaatacggccgggagagatgaacatatatacagtactcataattattaaccatgcatgggcattaataaaagtagtactccgtatatagcttccaagcaaggctacatactagaatcatactgtatcttgattgccagaggtgcaatcatgcatagacatactt

SEQ ID NO: 30 - DNA, 1212 bp, Oryza sativa
tctactacacacgaattgaattcaaatggtactaggtacaagtactccactagtacgaccatgatgtttcccacctgaaagcaacacagaatacgttcgtacgtacacgtacagtaaaaccacaataatgcacaagagtagggatcgatggagagtacatgcaattgctcatcacttgtcaccatcactggttcaaatttgcctactctagatctcgatcgtggtactgcttctaaccataatgatcggtacatgttcaaattcgaaactcaaaaaatgtgtgtacaatacggccgggagagatgaacatatatacagtactcataattattaaccatgcatgggcattaataaaagtagtactccgtatatagcttccaagcaaggctacatactagaatcatactgtatcttgattgccagaggtgcaatcatgcatagacatacttaagatcagggctagctagttcaggtgatgaacacatgctttgtaccccttgtaggatgcatggcaccaagtcgacattggagtacacacaccctggccctgctgcacgtgtagataagagtaccacctatcatcatcaatcacgcaatatggtctactgtgtctctttgattgggtgtaatataatatcggccactgtaaaaatttaaaatttaaaacttagtaattttaatttcgaagttgattttatgatatgctcaacgtatctcttttttttcaagctagcgttggtctttaaaagtgcgtttgtttataaggttagggtgagacttttagcttgtgtttggttagtgggatatggaatgagatgggttaggtcgatcctatttttcgagctgtttgggtagagggatgtgtaggacgggatgatcctagatgggaatattcttcccagatccaggacgaggtggtccgccaaaatcggctggactaatccatcccactttgatggagcgaatggcattagctccttccgcctcctgctcggctccgccactggccgcccgttcctcctcctccgtctgctccctcccacggcgtccgtccctcctcctcttcctgctccctcccccgatgccgctccgtcctcctccgcctgctccctcccccggcgcccgtccctcctcctctgcccgctcgccaagttgccggctgcaccgcccgctcgtgcagctcctcctgccatccgcgccgccggtcgctga

SEQ ID NO: 31 - DNA, 367 bp, Oryza sativa
ttaagggcccctttgaatcaaaggatttatgtaggaatttcataggattcaaatcctataggaaattttcctatttggccctttaattcaaaggattgaagctttccaaatcctatgaaattcctatggaatgacacattgcatgtagattttggaggaaatttagcaagagctccaacctcttggaaaatttcctttgagtctatctctctcatccgattcctgcgtttttcctgcactccaatcaaacgaccattcctgtgtttttcctgtgttttgcaatcctctgttttacacttcaattcctgtcagaatcctatgtttttcctattcctccgttttttctaccctgcgattcaaaggggcc

SEQ ID NO: 32 - DNA, 1580 bp, Oryza sativa
atgggcaaggcggcgcggtggttccgcaacatgtggggaggagggaggaaggagcagaagggcgaggcgccggcgagtggggggaagaggtggagcttcgggaagtcgtcgagggactcggcggaggccgcggcggctgctgctgcggcggcggcggaggcttccgggggcaatgcggcgatcgccagggcggccgaggcggcgtggctcaggtcggtgtacgccgacacggagcgggagcagagcaagcacgccatcgccgtcgccgcggccaccgcggcggcggctgatgccgccgtggcggccgctcaggccgccgtcgccgtcgtgcggcttactagcaagggccgctcggctcccgtcctcgccgccaccgtcgccggcgacacgcgcagccttgccgccgccgccgtcagaatccagacggcattcagaggcttcctggtaagcaacgggtgctcgtcttcttgctctggtttcgtcgtcgtcgtcgtcgccattgtcgttaatcatggcgtgtgttcgtgcaggcgaagaaggcgctgcgagcgctcaaggcgctggtgaagctgcaggcgctggtgcgcggctacctcgttcgccggcaggccgccgccacgctgcagagcatgcaggcgctcgtccgcgcccaggccactgtccgcgcccaccgcagtggcgccggcgccgccgccaatctcccgcacctccaccacgctcccttctggccccgccgctcgctggtacgccgctggctaaatctcgccgacgacatcgccatgtatatgttcgatgttgacgttgtgtgttggcgatggatgcagcaggagaggtgcgccggcgacgacacgaggagcgagcacggtgtggcggcgtacagccggcggctgtcggcgagcatcgagtcgtcgtcgtacgggtacgaccggagccccaagatcgtggaggtggacaccgggaggcccaagtcgcggtcgtcgtcgtcgcggcgggcgagctccccgctgctgctcgacgccgctgggtgcgcgagcggcggcgaggactggtgcgccaactccatgtcgtcgccgctcccgtgctacctccccggcggcgcgccgccgccccgcatcgccgtcccgacgtcgcgccacttccccgactacgactggtgcgcgctggagaaggcccggccggcgacggcgcagagcacgccgcggtacgcgcacgcgccgccgacgccgaccaagagcgtgtgcggcggcggcggcggcggcggcatccactcgtcgccgctcaactgcccgaactacatgtccaacacgcagtcgttcgaggcgaaggtgcgttcgcagagcgcgccgaagcagcggccggagaccggcggcgccggcgccggcggcggccggaagcgggtgccgctgagcgaggtggtggtggtggagtccagggcgagcttgagcggcgtgggcatgcagcgctcgtgcaaccgggtgcaggaggcgttcaacttcaagacggccgtcgtcggccgcctcgaccgctcgtcggagtccggcgagaacgaccgccacgcgttcttgcagaggaggtggtga

SEQ ID NO: 33 - DNA, 4101 bp, Artificial Sequence
  Cas9 sequence
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 34 - DNA, 24 bp, Artificial Sequence
  Arabidopsis thaliana fwd primer
ggcaacagaacaaacacgcgattg

SEQ ID NO: 35 - DNA, 24 bp, Artificial Sequence
  Arabidopsis thaliana rev primer
aaaccaatcgcgtgtttgttctgt

SEQ ID NO: 36 - DNA, 24 bp, Artificial Sequence
  Glycine max fwd primer
ggcaaggctgcggtggcggttgtt

SEQ ID NO: 37 - DNA, 24 bp, Artificial Sequence
  Glycine max rev primer
aaacaacaaccgccaccgcagcct

SEQ ID NO: 38 - DNA, 24 bp, Artificial Sequence
  Medicago truncatula fwd primer
ggcattctcaaagagagattcgag

SEQ ID NO: 39 - DNA, 24 bp, Artificial Sequence
  Medicago truncatula rev primer
aaacctcgaatctctctttgagaa

SEQ ID NO: 40 - DNA, 24 bp, Artificial Sequence
  Triticum aestivum fwd primer
ggcacagcaaagggccgacgtcga

SEQ ID NO: 41 - DNA, 24 bp, Artificial Sequence
  Triticum aestivum rev primer
aaactcgacgtcggccctttgctg

SEQ ID NO: 42 - DNA, 24 bp, Artificial Sequence
  Zea mays fwd primer
ggcaccgcgtgcgccgagacgcac

SEQ ID NO: 43 - DNA, 24 bp, Artificial Sequence
  Zea mays rev primer
aaacgtgcgtctcggcgcacgcgg

SEQ ID NO: 44 - DNA, 24 bp, Artificial Sequence
  Oryza sativa fwd primer
ggcacgaggcggcgtggctcaggt

SEQ ID NO: 45 - DNA, 24 bp, Artificial Sequence
  Oryza sativa rev primer
aaacacctgagccacgccgcctcg

SEQ ID NO: 46 - DNA, 24 bp, Artificial Sequence
  Sorghum bicolor fwd primer
ggcagtcgagtcctcgtcgtacgg

SEQ ID NO: 47 - DNA, 24 bp, Artificial Sequence
  Sorghum bicolor rev primer
aaacccgtacgacgaggactcgac

SEQ ID NO: 48 - DNA, 23 bp, Artificial Sequence
  Sorghum bicolor target sequence
gtcgagtcctcgtcgtacggcgg

SEQ ID NO: 49 - DNA, 1422 bp, Setaria italica
atgggcaaggcggcgcggtggttccgcagcttcctgggcaagaaggagcaggccagtaaagaccagaggcggcagcaggaccagccgccgcccccgccggccaccgccaagcgctggagcttcggcaagtcctcccgggactcggcggaggccgccgcggccgcggccgcgggcgccgtgtcggccggctcgggcaacgcggcgatcgcgcgcgcggcggaggccgcgtggctcaggtccgcggcctacgacgagacgaacagggagcgggagcagagcaagcacgccatcgccgtggccgcggccactgcggcggcggcggacgcggcggtggccgcggcccaggcggccgtcgccgtcgtgcggctcaccagcaagggccgcgccgcgcccaccctcgccaccgccgccggcggccgcgccgctgccgccgtcaggatccagacggcgttccgaggattcttggcgaagaaggcgttgcgcgcgctcaaggcgcttgtgaagctgcaggcgctggtgcggggctacctcgtgcgcaggcaggcggccgccacgctccacagcatgcaggccctcgtccgcgctcaggccaccgtgcgcgcgcaccgcgccggcgtcccagtcgtcttcccgcacctccaccacccgcccgtccggccgcgctactcgctgcaagagcggtacgccgacgacacgcggagcgagcacggcgcgccggcgtacggcagccggcggatgtcggcgagcgtcgagtcctcgtcgtacgcgtacgaccggagccccaagatcgtggaggtggacccggggcggcccaagtcgcgctcatcctcgcgtcgcgcgagctccccgctggtcgacgccggcagcagcggtggcgaggagtggtgcgctaactccgcgtgctcgccgctgccgtgctacctgtccggcggcccgccgcagccgccgcgcatcgccgtgccaacctcgcgccagttcccggactacgactggtgcgcgctggagaaggcgcggccggcgacggcgcagaacacgccgcggtacctgcacgtgcacgcgcacgcgccggccaccccgaccaagtccgtggcgggctactcgccgtcgctcaacggctgccggaactacatgtcgagcacgcaggcttcggaggcgaaggtgcggtcgcagagcgcgccgaagcagcggccggagctcgcctgcggcggcggcgctcggaagcgggtgccgctgagcgaggtggtggtggtggagtcgtcccgcgcgagcctgagcggcgtcgtcggcatgcagcgcgggtgcggcggccgcgcgcacgaggcgttcagcttcaagtccgccgtcgtcggccgcatcgaccgcacgctggaggtggccggcgtcgagaacgaccgcctggcgttcctgcagaggaggtggtga

SEQ ID NO: 50 - protein, 473 aa, Setaria italica
MGKAARWFRSFLGKKEQASKDQRRQQDQPPPPPATAKRWSFGKSSRDSAEAAAAAAAGAVSAGSGNAAIARAAEAAWLRSAAYDETNREREQSKHAIAVAAATAAAADAAVAAAQAAVAVVRLTSKGRAAPTLATAAGGRAAAAVRIQTAFRGFLAKKALRALKALVKLQALVRGYLVRRQAAATLHSMQALVRAQATVRAHRAGVPVVFPHLHHPPVRPRYSLQERYADDTRSEHGAPAYGSRRMSASVESSSYAYDRSPKIVEVDPGRPKSRSSSRRASSPLVDAGSSGGEEWCANSACSPLPCYLSGGPPQPPRIAVPTSRQFPDYDWCALEKARPATAQNTPRYLHVHAHAPATPTKSVAGYSPSLNGCRNYMSSTQASEAKVRSQSAPKQRPELACGGGARKRVPLSEVVVVESSRASLSGVVGMQRGCGGRAHEAFSFKSAVVGRIDRTLEVAGVENDRLAFLQRRW

SEQ ID NO: 51 - DNA, 23 bp, Artificial Sequence
  Setaria italica target sequence
cgtggccgcggccactgcggcgg

SEQ ID NO: 52 - DNA, 20 bp, Artificial Sequence
  Setaria italica protospacer sequence
cgtggccgcggccactgcgg

SEQ ID NO: 53 - DNA, 24 bp, Artificial Sequence
  Setaria italica fwd primer
ggcacgtggccgcggccactgcgg

SEQ ID NO: 54 - DNA, 24 bp, Artificial Sequence
  Setaria italica rev primer
aaacccgcagtggccgcggccacg

SEQ ID NO: 55 - DNA, 2146 bp, Oryza sativa
gcgtttccctctcttattcaaacttgacccgtttcgccttcttgctcaagtgttcgacctggtcttggagcgcggcgtgtctctctcgccggccggagtcgcgaattccggccatgggcaaggcggcgaggtggttccgcagcctgtggggcggcggcggcgggaagaaggagcaggggagagaacatgggaggacggccgcggcgccgcccccgccggacaggaagcggtggagcttcgccaagtcgtcgagggactcgacggagggggaggcggcggcggcggtgggagggaatgcggcgatcgcgaaggcggccgaggcggcgtggctcaagtcgatgtacagcgacaccgagagggagcagagcaagcacgccatcgcggtcgccgcggcgaccgcggctgcggcggacgcggccgtggcggcggcacaggcggccgtcgaggtcgtccgcctcaccagccaggggccacccacctcgtcggtgttcgtctgcggcggcgtcttggatccccgtggccgcgccgccgcggtcaagatccagacagccttccgaggattcttggtgagtgagccccaacaacttcctcacttcttccaagaacaacagtgtctgcttctgttcttgatctgttcgtcttctttggcgacgtgctcatttcgatttcatccactgttccagtagatttccttttccaaaaaaagctcatagattaagacatgattagatttttatttttgttcttggttcaggcgaagaaggcgctgcgagcgctcaaggcgctggtgaagctgcaggcgctggtgcgcggctacctggtgaggcggcaggcggcggcgacgctgcagagcatgcaggcgctcgtccgcgcgcaggccgccgtccgcgccgcgcgctcgtcgcgcggcgccgcgctgccgccgctgcacctccaccaccaccctcccgtccggccgcgctattccctggtacgagtacgaccacgatcgcttgcgtgcgaagcgggcgagctttttttttaaaggtgttcgtccgaggcatgttggttgctgtgacacaattcttacctcgggggtttcttgtgtttgcagcaagagcggtatatggacgacacgaggagcgagcatggcgtggcggcgtacagccgccgcctgtcggcgagcatcgagtcgtcgtcgtacgggtacgaccggagccccaagatcgtggagatggacaccgggcggcccaagtcgaggtcgtcgtcggtcaggacgagccctcccgtggtcgacgccggcgccgccgaggagtggtacgccaactcggtgtcgtcgccgctcctcccgttccaccagctccccggcgcgccgccgcggatatcggcgccgagcgcacgccacttcccggagtacgactggtgcccgctcgagaagcccaggccggcgacggcgcagagcacgccgcggcttgcgcacatgccggtgacgccgacgaagagcgtctgcggcggcggcggctacggcgcgtcgcccaactgccgcggctacatgtcgagcacgcaatcgtcggaggcgaaggtgcggtcccagagcgcgccgaagcagcggccggagccgggcgtcgccggcggcaccggcggcggcgcgcggaagagggtgccgctgagcgaggtgaccctggaggcgagggcgagcctgagcggcgtgggcatgcagcgctcgtgcaaccgtgtccaggaggcgttcaacttcaagaccgccgtgctcagccgcttcgaccgctcgtcggagccggccgccgagagggaccgcgacctcttcttgcagaggaggtggtgatctgaacagcgttcgccattgcaagaaggaagaggactacaagaactagttcttcttcttcttcttagtctctgtttctatgcgacatagtagcgatcgatcatgtttgatcgatggcaatggcgatcgtgtgctccgccattgccgtcgtctccgagcttgttactgacaagtgacaggcaaagtgtacgttgagctagctggaggggagattacaaaaaaaaaaaatcccacttctttcccctctgatttaacagtgcacttggatgtacattcccctatcaattcaaggccagcaaatcaaatcccgttgtttttttttaa

SEQ ID NO: 56 - DNA, 1398 bp, Oryza sativa
atgggcaaggcggcgaggtggttccgcagcctgtggggcggcggcggcgggaagaaggagcaggggagagaacatgggaggacggccgcggcgccgcccccgccggacaggaagcggtggagcttcgccaagtcgtcgagggactcgacggagggggaggcggcggcggcggtgggagggaatgcggcgatcgcgaaggcggccgaggcggcgtggctcaagtcgatgtacagcgacaccgagagggagcagagcaagcacgccatcgcggtcgccgcggcgaccgcggctgcggcggacgcggccgtggcggcggcacaggcggccgtcgaggtcgtccgcctcaccagccaggggccacccacctcgtcggtgttcgtctgcggcggcgtcttggatccccgtggccgcgccgccgcggtcaagatccagacagccttccgaggattcttggcgaagaaggcgctgcgagcgctcaaggcgctggtgaagctgcaggcgctggtgcgcggctacctggtgaggcggcaggcggcggcgacgctgcagagcatgcaggcgctcgtccgcgcgcaggccgccgtccgcgccgcgcgctcgtcgcgcggcgccgcgctgccgccgctgcacctccaccaccaccctcccgtccggccgcgctattccctgcaagagcggtatatggacgacacgaggagcgagcatggcgtggcggcgtacagccgccgcctgtcggcgagcatcgagtcgtcgtcgtacgggtacgaccggagccccaagatcgtggagatggacaccgggcggcccaagtcgaggtcgtcgtcggtcaggacgagccctcccgtggtcgacgccggcgccgccgaggagtggtacgccaactcggtgtcgtcgccgctcctcccgttccaccagctccccggcgcgccgccgcggatatcggcgccgagcgcacgccacttcccggagtacgactggtgcccgctcgagaagcccaggccggcgacggcgcagagcacgccgcggcttgcgcacatgccggtgacgccgacgaagagcgtctgcggcggcggcggctacggcgcgtcgcccaactgccgcggctacatgtcgagcacgcaatcgtcggaggcgaaggtgcggtcccagagcgcgccgaagcagcggccggagccgggcgtcgccggcggcaccggcggcggcgcgcggaagagggtgccgctgagcgaggtgaccctggaggcgagggcgagcctgagcggcgtgggcatgcagcgctcgtgcaaccgtgtccaggaggcgttcaacttcaagaccgccgtgctcagccgcttcgaccgctcgtcggagccggccgccgagagggaccgcgacctcttcttgcagaggaggtggtga

SEQ ID NO: 57 - protein, 465 aa, Oryza sativa
MGKAARWFRSLWGGGGGKKEQGREHGRTAAAPPPPDRKRWSFAKSSRDSTEGEAAAAVGGNAAIAKAAEAAWLKSMYSDTEREQSKHAIAVAAATAAAADAAVAAAQAAVEVVRLTSQGPPTSSVFVCGGVLDPRGRAAAVKIQTAFRGFLAKKALRALKALVKLQALVRGYLVRRQAAATLQSMQALVRAQAAVRAARSSRGAALPPLHLHHHPPVRPRYSLQERYMDDTRSEHGVAAYSRRLSASIESSSYGYDRSPKIVEMDTGRPKSRSSSVRTSPPVVDAGAAEEWYANSVSSPLLPFHQLPGAPPRISAPSARHFPEYDWCPLEKPRPATAQSTPRLAHMPVTPTKSVCGGGGYGASPNCRGYMSSTQSSEAKVRSQSAPKQRPEPGVAGGTGGGARKRVPLSEVTLEARASLSGVGMQRSCNRVQEAFNFKTAVLSRFDRSSEPAAERDRDLFLQRRW

SEQ ID NO: 58 - DNA, 2506 bp, Zea mays
agccacgacgtcactgccgccattagacacatcaccgccagcagcagcgccagcaccttcgtcgcgccttttgaactgatcctgccgctttttgaactcatcgtccacggcgcacgcaccaactcaaaaaaacctcttggatttgggacggcgacgcccatttcttttttctttaatcccgtcgtccatctcgtgcctttgcgcagccagctactagggcgtcagctaagtcggtttacacgccgcacaaaaaacacacgaatattcagctagtagcagcgtgaggagaggagagggtcccccgacccgtcgtccatctcgtgcctttgcgcagccagctactagggcgtcagctaagtcggtttacacgccgcacaaaaaacacacgaatattcagctagtagcagcgtgaggagaggagagggtcccccgacccgtcgtctctgccttcttgcgctgcatctttccgggtgctcttgtccgctaatggcccgccctcctctcgttttattccaaacccgctcctcccctgctttccctctcttattcaaactcgcagtcccagtcccaggctccatttttctaactccaccggccgttgccaccccctcacttcagctgcttctagttctaccgcacctcagtgactcagtcccccgctaggctcggagcggagcggagccgagcttcacttgccggctgcgaattccggggatgggcaaggcggcgaggtggctccgcggcctgctcggcggcgggaggaaagaccaggagaggcgggcctcgccggcgccgcccaccgcggacaggaagcgctggagcttcgcgaggtcctcgcgggactcggccgaggccgccgcggcggcgaccgagggctccgtgcgcggcggtggcaacgcggcgatcgcgcgggccgccgaggccgcgtggctcaagtcgctctacgacgacacggggcggcagcagagcaagcacgccatcgccgtcgccgcggccacagcggcggcggcggacgcggccgtggccgccgcgcaggccgcggtcgaggtcgtccggctcaccagccagggcccggtcttcggcggcggagggccggtgcccgtgctggacccccgcggccgcgccggcgccgccgtcaagatccagacggcattcagacgcttcttggcgaagaaggcgctgcgagcgctgaaggcgctggtgaagctgcaggcgctggtgcgcggctacctggtgcggcggcaggcggcggcgacgctgcagagcatgcaggcgctcgtccgcgcgcaggcagccgtccgcgccgcccgctacagccgcgcgctacccgcgctcccgcccctccaccaccaccctcccgtccgcgcgcgcttctcgctgcaagagcggtacggtgacgacacgcgcagcgagcacggcgtggcggcgtacagccggcgcttgtccgcgagcatcgagtcggcgtcgtacggaggcgggtacgaccggagccccaagatcgtggagatggacacggcgcggccccggtcgcgcgcgtcgtccctgcgcaccgaggacgagtggtacgcgcagtcggtgtcgtcgccgctcctgccgccgccgccgccgccgccgtgccagcacctgcaccagtaccaccacctgcccccgcgcatcgcggtgcccacgtcgcgccacttcccggactacgactggtgcgcgccggagaagccgcggccggcgacggcgcagtgcacgccccgctgcgcgccgccgacccccgccaggagcgtctgcggcgccgggggcaacggcggcggctacctcgccgcgtcgcccggctgtcccgggtacatgtcgagcacgcggtcgtcggaggccaagtcgtcgtcccggtcgcagagcgcgccgaagcagcggccgctggagcagcaggagcagcagcagcagccggcccggaagcgggtgccgctcagcgaggtggtcctggaggcccgcgcgagcctgggcggcgccggcgtgggcatgcacaagccgtgcaatacccgcgcgcaggcgcaggacgcgttcgacttccgcaccgccgtcgtgagccggttcgatcggcgcgcgtcggacgccgccgcggcggcggccgagcggcgggatcgcgaattgttcttcctgcagaggaggtggtgaaggtgaaccgatcgaccgcccgaccgggatgattaatcgggtgctgctaatagggaaggctctcattaattccttttcagcatgcatctcctcgctgatccctgttgttccgatcccatccgtgacctctcactgctgtcgttcttcctgcttgcaataagctagtgtgtgtcggggaagtagggagagatttcatcccgtcccgtaccgtttgatttcgtttttgcgttcataaacagtagcgcggctggatcgtcatcatctcgatccatgcatgtacattccgcctgttccccaatcaccatcaatcaacaagaaagagaagcacggtgtcgtttccgagcca

SEQ ID NO: 59 - DNA, 1446 bp, Zea mays
atgggcaaggcggcgaggtggctccgcggcctgctcggcggcgggaggaaagaccaggagaggcgggcctcgccggcgccgcccaccgcggacaggaagcgctggagcttcgcgaggtcctcgcgggactcggccgaggccgccgcggcggcgaccgagggctccgtgcgcggcggtggcaacgcggcgatcgcgcgggccgccgaggccgcgtggctcaagtcgctctacgacgacacggggcggcagcagagcaagcacgccatcgccgtcgccgcggccacagcggcggcggcggacgcggccgtggccgccgcgcaggccgcggtcgaggtcgtccggctcaccagccagggcccggtcttcggcggcggagggccggtgcccgtgctggacccccgcggccgcgccggcgccgccgtcaagatccagacggcattcagacgcttcttggcgaagaaggcgctgcgagcgctgaaggcgctggtgaagctgcaggcgctggtgcgcggctacctggtgcggcggcaggcggcggcgacgctgcagagcatgcaggcgctcgtccgcgcgcaggcagccgtccgcgccgcccgctacagccgcgcgctacccgcgctcccgcccctccaccaccaccctcccgtccgcgcgcgcttctcgctgcaagagcggtacggtgacgacacgcgcagcgagcacggcgtggcggcgtacagccggcgcttgtccgcgagcatcgagtcggcgtcgtacggaggcgggtacgaccggagccccaagatcgtggagatggacacggcgcggccccggtcgcgcgcgtcgtccctgcgcaccgaggacgagtggtacgcgcagtcggtgtcgtcgccgctcctgccgccgccgccgccgccgccgtgccagcacctgcaccagtaccaccacctgcccccgcgcatcgcggtgcccacgtcgcgccacttcccggactacgactggtgcgcgccggagaagccgcggccggcgacggcgcagtgcacgccccgctgcgcgccgccgacccccgccaggagcgtctgcggcgccgggggcaacggcggcggctacctcgccgcgtcgcccggctgtcccgggtacatgtcgagcacgcggtcgtcggaggccaagtcgtcgtcccggtcgcagagcgcgccgaagcagcggccgctggagcagcaggagcagcagcagcagccggcccggaagcgggtgccgctcagcgaggtggtcctggaggcccgcgcgagcctgggcggcgccggcgtgggcatgcacaagccgtgcaatacccgcgcgcaggcgcaggacgcgttcgacttccgcaccgccgtcgtgagccggttcgatcggcgcgcgtcggacgccgccgcggcggcggccgagcggcgggatcgcgaattgttcttcctgcagaggaggtggtga

SEQ ID NO: 60 - protein, 481 aa, Zea mays
MGKAARWLRGLLGGGRKDQERRASPAPPTADRKRWSFARSSRDSAEAAAAATEGSVRGGGNAAIARAAEAAWLKSLYDDTGRQQSKHAIAVAAATAAAADAAVAAAQAAVEVVRLTSQGPVFGGGGPVPVLDPRGRAGAAVKIQTAFRRFLAKKALRALKALVKLQALVRGYLVRRQAAATLQSMQALVRAQAAVRAARYSRALPALPPLHHHPPVRARFSLQERYGDDTRSEHGVAAYSRRLSASIESASYGGGYDRSPKIVEMDTARPRSRASSLRTEDEWYAQSVSSPLLPPPPPPPCQHLHQYHHLPPRIAVPTSRHFPDYDWCAPEKPRPATAQCTPRCAPPTPARSVCGAGGNGGGYLAASPGCPGYMSSTRSSEAKSSSRSQSAPKQRPLEQQEQQQQPARKRVPLSEVVLEARASLGGAGVGMHKPCNTRAQAQDAFDFRTAVVSRFDRRASDAAAAAAERRDRELFFLQRRW

SEQ ID NO: 61 - DNA, 2267 bp, Sorghum bicolor
cattttctttaatccccgtcgtccatctcgtgcctttgccgttgctacttgcattggtagggcatcatcagtcagccagtttacacgtcgcaccaaaaacacacgaacattcagatcagctagtagcttgagagtgagagggtccccccgtcgtctctgccttcttgcgctgcatctttccgggcgggacatctggtgctcttgtccgctaatggccccccaccccctcctttctttttattccaaacccgctcctcccctgctttccctctcttattcaaactcgcagtcacagcctccctctttttctaacgccaccggccgttgccacccctcatcacaccctcacttcagcacttcagctgcagctcagtaccgctaggcttggagcgcgcaccggcgcggagcagacagcggagcctctcttgccgtccggctgcgaattccggggatgggcaaggcggcgcggtggttccgcagcttgcttggcggcgggaggaaggaccaggagaggcagcgggcctcgccggcgccgccgcccaccgcggacaggaagcgctggagcttcgctcgctcgtcgcgggactcggccgaggccgcggcggcggcgaccgagggctccgtgcggggcggtgccgccgccgccggcggtaacgcggcgatcgcgagggcggccgaggccgcgtggctcaagtcgctctacgacgacacggggcggcagcagagcaagcacgccatcgccgtcgctgcggctaccgcggcggcggcggacgcggccgtggccgccgcgcaggccgccgtcgaggtcgtccggctcaccagccagggccctgtcttcggcggcggaggtggaggaggggccgtgctcgacccccgtggccgcgccggcgccgccgtcaagatccagacggccttcagaggcttcttggcgaagaaggcgctgcgagcgctcaaggcgctggtgaagctgcaggcgctggtgcgcggctacctggtgcggcggcaggcggcggcgacgctgcagagcatgcaggcgctggtccgcgcgcaggccaccgtccgcgccgcccgcggctgccgcgccctgccctcgctcccgccgctccaccacccagctgcattccgcccgcgcttctcgctgcaagagcggtacgctgacgacacgcgcagcgagcacggcgtggcggcgtacagccggcgcctgtccgcgagcatcgagtcggcgtcgtacgggggcggcgggtacgaccggagccccaagatcgtggagatggacacggcgcggccgaggtcccgcgcgtcgtcccttcgcaccgaagacgagtggtacgcgcagtcggtgtcgtcgccgctgcagccgccgtgccaccacctgccgccgcgcatcgcggtgccgacgtcgcgccacttcccggactacgactggtgcgcgccggagaagccccggccggcgacggcgcagtgcacgccccggttcgcgccgccgaccccggcaaagagcgtctgcggcggcggcggtggtaacggcggctactacgcccaccacctcgccgcggggtcgcccaactgccccgggtacatgtcgagcacgcagtcgtcggaggccaagtcgtcgtcccggtcgcacagcgcgccgaagcagcggccgccggagcagcagcagccgtcccggaagcgcgtgccgctgagcgaggtggtcctggaggcccgcgccagcctgggcggcgtcggcgtcggtatgatgcacaagccgtgcaacacccgcgccgcgcagccgcaggagccgttcgatttccgcgccgccgtcgtcagccggttcgagcagcgcgcgtcggacgccgctgccgccgccgaacgggaccgcgacgtgttgttcctgcagaggaggtggtgaaggtgaaccgaccgatcgatcgatcggtcagtgagttagtcgaagtgctccgcctgcctgagtgagattatggcctagtatgattaatcggtgctgctaatagggattgttaattaggttctcattaattcctcgcccttttgtgatctctgttagttcttccgatcgcgtccatgacctctctctgcagtcggccattcttcctgcttgcaataagctagtgtgtgtgtgtgtgtgaagtagggagagatttcatcatcccgttccgtttcgtttttttcgttcataaaaacagtagtgcagctggatcatcagctcgatgtacattccgcctgttctccgatcatcaccatcaagaaagagagaaaaaaaa

SEQ ID NO: 62 - DNA, 1443 bp, Sorghum bicolor
atgggcaaggcggcgcggtggttccgcagcttgcttggcggcgggaggaaggaccaggagaggcagcgggcctcgccggcgccgccgcccaccgcggacaggaagcgctggagcttcgctcgctcgtcgcgggactcggccgaggccgcggcggcggcgaccgagggctccgtgcggggcggtgccgccgccgccggcggtaacgcggcgatcgcgagggcggccgaggccgcgtggctcaagtcgctctacgacgacacggggcggcagcagagcaagcacgccatcgccgtcgctgcggctaccgcggcggcggcggacgcggccgtggccgccgcgcaggccgccgtcgaggtcgtccggctcaccagccagggccctgtcttcggcggcggaggtggaggaggggccgtgctcgacccccgtggccgcgccggcgccgccgtcaagatccagacggccttcagaggcttcttggcgaagaaggcgctgcgagcgctcaaggcgctggtgaagctgcaggcgctggtgcgcggctacctggtgcggcggcaggcggcggcgacgctgcagagcatgcaggcgctggtccgcgcgcaggccaccgtccgcgccgcccgcggctgccgcgccctgccctcgctcccgccgctccaccacccagctgcattccgcccgcgcttctcgctgcaagagcggtacgctgacgacacgcgcagcgagcacggcgtggcggcgtacagccggcgcctgtccgcgagcatcgagtcggcgtcgtacgggggcggcgggtacgaccggagccccaagatcgtggagatggacacggcgcggccgaggtcccgcgcgtcgtcccttcgcaccgaagacgagtggtacgcgcagtcggtgtcgtcgccgctgcagccgccgtgccaccacctgccgccgcgcatcgcggtgccgacgtcgcgccacttcccggactacgactggtgcgcgccggagaagccccggccggcgacggcgcagtgcacgccccggttcgcgccgccgaccccggcaaagagcgtctgcggcggcggcggtggtaacggcggctactacgcccaccacctcgccgcggggtcgcccaactgccccgggtacatgtcgagcacgcagtcgtcggaggccaagtcgtcgtcccggtcgcacagcgcgccgaagcagcggccgccggagcagcagcagccgtcccggaagcgcgtgccgctgagcgaggtggtcctggaggcccgcgccagcctgggcggcgtcggcgtcggtatgatgcacaagccgtgcaacacccgcgccgcgcagccgcaggagccgttcgatttccgcgccgccgtcgtcagccggttcgagcagcgcgcgtcggacgccgctgccgccgccgaacgggaccgcgacgtgttgttcctgcagaggaggtggtga

SEQ ID NO: 63 - protein, 480 aa, Sorghum bicolor
MGKAARWFRSLLGGGRKDQERQRASPAPPPTADRKRWSFARSSRDSAEAAAAATEGSVRGGAAAAGGNAAIARAAEAAWLKSLYDDTGRQQSKHAIAVAAATAAAADAAVAAAQAAVEVVRLTSQGPVFGGGGGGGAVLDPRGRAGAAVKIQTAFRGFLAKKALRALKALVKLQALVRGYLVRRQAAATLQSMQALVRAQATVRAARGCRALPSLPPLHHPAAFRPRFSLQERYADDTRSEHGVAAYSRRLSASIESASYGGGGYDRSPKIVEMDTARPRSRASSLRTEDEWYAQSVSSPLQPPCHHLPPRIAVPTSRHFPDYDWCAPEKPRPATAQCTPRFAPPTPAKSVCGGGGGNGGYYAHHLAAGSPNCPGYMSSTQSSEAKSSSRSHSAPKQRPPEQQQPSRKRVPLSEVVLEARASLGGVGVGMMHKPCNTRAAQPQEPFDFRAAVVSRFEQRASDAAAAAERDRDVLFLQRRW

SEQ ID NO: 64 - DNA, 1890 bp, Medicago truncatula
ctcaaacactacaaccaatgggtagaaccataaggtggttcaagagtttgtttgggataaagaaagacagagataattcaaactcaaattcttcaagtaccaaatggaatccttctcttcctcatcctccttctcaagatttctcaaagagagattcgagaggcttgtgtcataatccagctaccatacctcccaacatttcacctgcagaagctgcttgggttcaatccttctactcagaaactgagaaggagcaaaacaagcacgccattgcggtagcagctgcaacagcagcagccgcagatgctgctgtggcagctgctcaagctgccgtgggctgtggttagattaaccagccacggcagagacaccatgtttggtggtggacaccagaaatttgctgctgtcaagattcaaacaacatttaggggttacttggtaagtttgattcacttttctttaattaattatgtgattttactaatgcagttctaagaaaaacagattttgctcaaattgaactagtcagattcaaattctaggctttttatgttttaaagtattattatagcttcaattagaactaaagaaagtgttaatgaacttggtttgatgtttatatatttctatttttcttgtggcattgtggaatgtgagagaatttgaaattggtttttctataataggcaagaaaagcactaagagccttaaagggattggtaaagttacaagcactagtgagagggtacttagtgaggaagcaagcaacagcaacattacacagtatgcaagctctaattagagcacaagcaacagtaaggtctcataaatctcgtggactcatcataagcacaaagaatgaaacaaataacagatttcaaacacaagctagaagatccacggtaaaatacataaacagatctcatattttaattcctagtatcacttgaatatttacatttcttatgattgttattttgcaggaaaggtataatcacaatgagagtaacaggaacgagtacacagcttcaattcctattcacagcagaagattatcatcatcttttgatgctacaatgaacagttatgatattggaagtccaaaaatagtagaagttgatactggaagaccaaaatcaaggtctagaagaagcaatacatcaatttcagattttggagatgacccttcatttcaaacactttcttctccacttcaagttactccatctcagttatacattccaaatcaaagaaattataacgaatcagattggggaataacaggtgaagaatgcagattttcaactgcacagagcactccacgtttcacaagttcatgtagttgtggatttgttgcaccttccacacctaaaacaatttgtggagatagtttttacattggtgattatggtaattatcctaattacatggctaatacacagtcttttaaggctaaattgaggtctcatagtgctccaaagcaacgacctgaaccaggtccgaagaagaggctttcattgaatgaattgatggaatctagaaacagtttgagtggagttagaatgcagaggtcttgttcacagattcaggatgctattaattttaagaatgctgtgatgagtaaacttgataagtccactgattttgatagaaacttttcaaagcagaggaggttgtgatccgaggagcaatgccgtgtgtccggtgtccatgtcagagtccatgcttcaaagtggtgattgattagtgagtttaagaagcatttatgagagcgctagtaaattgattggtaatttgcagacatatagtgggtagggaacaggtaagtgaagacaaaattgaaggataattaataacaatgataaactacaagtttggtgatggaatttg

SEQ ID NO: 65 - DNA, 1290 bp, Medicago truncatula; identical to SEQ ID NO: 12

SEQ ID NO: 66 - protein, 429 aa, Medicago truncatula; identical to SEQ ID NO: 11

SEQ ID NO: 67 - DNA, 1624 bp, Triticum aestivum
atgggcaaggcggcgaggtggctgcgtggcttgctgggcggcggcggcaagaaggagcaggggaaggagcagaggcgcccggccacggcgccgcacggggacaggaagcgctggagcttctgcaagtccaccagggactcggcagaggcggaggcggcggccgcggccgcggcgctcagcggcaacgcggcgatcgcgcgcgcggccgaggcggcatggctcaagtccttgtacaacgagaccgagcgcgagcagagcaagcacgccatcgccgtcgccgcggccaccgcggcggcggcggacgcggctatggctgccgcacaggcagccgtggaggtcgtgcggctcaccagcaaagggccgacgtcgacggtgctcgccgacgccgtcgcggagccccacggccgtgcctccgccgcggtcaagatccagacggcgttccgtggcttcctggtgagtaatttccttcctaacagcggcgccatgatttccgcaggtttaagcgctgagtaaccaaatcaatgcgtgttgaattatcgcaggccaagaaggctctgcgcgcgctcaaggggctggtgaagctgcaggcgctggtgcgcggctacctggtgcggaagcaggcggcggccacgctgcagagcatgcaggcgctcgtccgcgcgcaggcctgcatccgcgctgcccgctcgcgcgccgcggcgctcccgacgaaccttcgcgtccaccccactcctgtccggccgcgctactcgttggtaagtgaccacgggtccacggccggcatcgcttgcgaccaaagcaatcgatctcaatgtctctgaccgtccgaggtcgcgttgttctagctagccgaccgtaacaaatgtgcgcgtgcgtggtttcttgcttgtgtctgcagcaagagcggtacagcaccacggaggattcccggagcgaccaccgcgtggcgccgtactacagccgccggctgtcggcgagcgtggagtcgtcgtcgtgctacggctacgaccggagccccaagatcgtggagatggacaccggccggcccaagtcgcgctcctcctcgctccggacgacctcccccggcgccagcgaggagtgctacgcccactcggtgtcgtcgccgctcatgccgtgccgagcgcccccgcggatcgcggcgcccaccgcgcgccacttcccggagtacgagtggtgcgagaaggcccggccggcgacggcgcagagcacgccccggtacacgagctacgcgccggtgacgccgaccaagagcgtgtgcggcggctacacctacagcaacagcccgtcgacgctcaactgccccagctacatgtcgagcacgcagtcgtccgtggcgaaggtgcgttcgcagagcgcgccgaagcagcggccggaggagggcgcggtacggaagagggtgccgctgagcgaggtgatcatcctgcaggaggcccgggcgagcctgggcggcggcgggggcacgcagaggtcgtgcaaccggccggcgcaggaggaggcgttcagcttcaagaaggccgtcgtgagccgcttcgaccgctcgtcggaggcggccgagagggaacgtgaccgggaccgggacttgttcctgcagaaggggtggtga

SEQ ID NO: 68 - DNA, 1392 bp, Triticum aestivum; identical to SEQ ID NO: 4

SEQ ID NO: 69 - protein, 463 aa, Triticum aestivum; identical to SEQ ID NO: 3

SEQ ID NO: 70 - DNA, 3073 bp, Glycine max
ggagagctccctttctttgaaccctttttactcaatgcccggtttgaaccaagctcacgggattgttgtcagtgggtatctttcatatgtactacacgttgctccagaatgttagagagagatagattttttttataatcaatagtttaagtgatgtggatgctaaggattcatgaggaacgtaccttaaagactcgctgagtacttatataattacttataaattatttactaataagattcttatattgataataaaatttaaatttacatttttgtataatttgtgttcgatccttacctgataaatacatagagcaacaaagtaataatattgcattttttttagcataataacatagtgcaaagtgagataaagagagaaagaggaattggttattgttggtagtactttggtagtgttattggagtgagtggggtgggaagggaattcggcgctctttctcttttttgaattcaagatcaggtaagaagctgtttgttttgttttgggtgttggggttggggggcttttccctatgattattattgtcacttatttcataggatttcacctttgtgctttgtctgtaattcttgtctctttactcaagagggggtgagattttgaagcgtaatggttactattttaatgttgtttttttttttcatttcatttgagcctctgctaactttctgtgaccgctttttgtcctctaatgcacttcttcacgaggtcctgtcagaggtttattttcaaaaaatgttccatctttatccattttttcgctcccctcactataatttttaaggtttgtttgtttgatgcctttcattttaccccgttttattaatcttttcaaccatcctgctctcatcacttctcttctgctagattctttacactttacattacaccctcttttgtttctttgtccctccaatttttaaaaccaagaattttcattttcacctgtcccttgtatattgaaccatctgttttcacttggttgctacacttgttcacttacacaagcaaaacccctctttatctttaaccaaaagaggcaaagtttggcttaactttggcaccgttttctcattccagatcgtgactgaaaaagttgtgtgaagttattattatggggaaagctagcaggtggttgaaggggttgttggggatgaagaaggagaaggaccacagtgacaattcaggctcattggctcctgacaagaaggagaagaaaaggtggagttttgccaagccaccaccttcttcagttcctgccactgataacaacaacacctggctcagatcctatatttctgagacagagaatgagcagaacaagcatgcaattgccgtggcagccgccaccgctgctgctgccgatgccgccgtggccgccgcacaggcggccgtggctgttgtgaggctcacaagccaggggagaggggcattgttcagtggaagcagggagaaatgggctgctgtgaagatccaaactttttttagaggctacttggtatgtgtcttgtctttttgtgatgtttcaaatcaaaattttggtgttgtttatgtgggtatgtatgtgtgtgtgtgtctgctttctttcattttgaaaatgttgtttggttaattgcattggttcttgtcttggacatatttattttattaggtttgtttaagtgaagtgttttgcttgattcctactcctttgtctcctctcaaaaattattttagattcttaatgaaacagtgcctttcactttctgtttgggtcattcaaactaccctttgcactttccattctccacgttagagttgacttgtcttgttattttgatgctcttttaattaaaccattttcaatatttgacaattctttatgtacattttgtcaatttcatggttttattaagttccattactatactagcacttttaatttaaacttttatgttgcttactttcaggcacggaaggctcttagagcactgaaaggattggttaagatacaagctcttgttagagggtatttggttagaaagagggctgctgcaactcttcacagtatgcaagctctaataagagctcaaactgctgttagaacacagcgagctcgtcgttccatgagcaaagaagacagatttctacctgaagttcttgcaagaaaacctgtggtaattaaaaactggttttctagtcctgaatgattaccaacttcactcattttattttatctatcagcaaacaatgtcttatttgcttggtgttgtgtcctttttgtactttttaggaacgatttgatgaaacaaggagtgaattccacagtaaaaggctacctacatcctatgaaacatccttgaatggttttgatgagagccccaagattgttgaaattgacacatacaagactcgatcgaggtctaggcgcttcacctctacaatgtctgagtgtggagaagacatgtcctgccatgcaatctcatcccctcttccttgtccggtccccggtcgaatctcggttcctgattgcagacacattcaagattttgattggtactacaacgtggatgagtgtagattctccactgctcatagcaccccgcgtttcacaaactatgtgagggctaatgctccagctacaccagccaagagtgtttgtggagacactttcttcagaccttgctccaatttccccaactacatggccaatactcagtcattcaatgcaaaactaaggtctcacagtgctccaaagcaaagacctgaacccaagaaaaggctctcactcaatgaaatgatggcagcaagaaacagcataagtggtgttagaatgcaaaggccatcatccaatttctttccagactcaagaagaatcctggaattttttacaatcacaaggaatatttgagaggcaattggagtctaaatgatgttagtaatatctagaatttgtttttttttttttcacttgtgcttacaacttacaaattcagggatgaaattctcaattctcaattctgcttgtgtacattcttttaattaaagagtttttttttttttttt

SEQ ID NO: 71 - DNA, 1209 bp, Glycine max
atggggaaagctagcaggtggttgaaggggttgttggggatgaagaaggagaaggaccacagtgacaattcaggctcattggctcctgacaagaaggagaagaaaaggtggagttttgccaagccaccaccttcttcagttcctgccactgataacaacaacacctggctcagatcctatatttctgagacagagaatgagcagaacaagcatgcaattgccgtggcagccgccaccgctgctgctgccgatgccgccgtggccgccgcacaggcggccgtggctgttgtgaggctcacaagccaggggagaggggcattgttcagtggaagcagggagaaatgggctgctgtgaagatccaaactttttttagaggctacttggcacggaaggctcttagagcactgaaaggattggttaagatacaagctcttgttagagggtatttggttagaaagagggctgctgcaactcttcacagtatgcaagctctaataagagctcaaactgctgttagaacacagcgagctcgtcgttccatgagcaaagaagacagatttctacctgaagttcttgcaagaaaacctgtggaacgatttgatgaaacaaggagtgaattccacagtaaaaggctacctacatcctatgaaacatccttgaatggttttgatgagagccccaagattgttgaaattgacacatacaagactcgatcgaggtctaggcgcttcacctctacaatgtctgagtgtggagaagacatgtcctgccatgcaatctcatcccctcttccttgtccggtccccggtcgaatctcggttcctgattgcagacacattcaagattttgattggtactacaacgtggatgagtgtagattctccactgctcatagcaccccgcgtttcacaaactatgtgagggctaatgctccagctacaccagccaagagtgtttgtggagacactttcttcagaccttgctccaatttccccaactacatggccaatactcagtcattcaatgcaaaactaaggtctcacagtgctccaaagcaaagacctgaacccaagaaaaggctctcactcaatgaaatgatggcagcaagaaacagcataagtggtgttagaatgcaaaggccatcatccaatttctttccagactcaagaagaatcctggaattttttacaatcacaaggaatatttga

SEQ ID NO: 72 - protein, 402 aa, Glycine max
MGKASRWLKGLLGMKKEKDHSDNSGSLAPDKKEKKRWSFAKPPPSSVPATDNNNTWLRSYISETENEQNKHAIAVAAATAAAADAAVAAAQAAVAVVRLTSQGRGALFSGSREKWAAVKIQTFFRGYLARKALRALKGLVKIQALVRGYLVRKRAAATLHSMQALIRAQTAVRTQRARRSMSKEDRFLPEVLARKPVERFDETRSEFHSKRLPTSYETSLNGFDESPKIVEIDTYKTRSRSRRFTSTMSECGEDMSCHAISSPLPCPVPGRISVPDCRHIQDFDWYYNVDECRFSTAHSTPRFTNYVRANAPATPAKSVCGDTFFRPCSNFPNYMANTQSFNAKLRSHSAPKQRPEPKKRLSLNEMMAARNSISGVRMQRPSSNFFPDSRRILEFFTITRNI

SEQ ID NO: 73 - DNA, 1918 bp, Arabidopsis thaliana
atatataaatggtgatacttatatttatttttgaagaacctatccatcacattctctctctcttttcttgaacttcttatctctctttttttttacagttctcctttctcaaaacatcacattgtgtcttcagtttcagtgccgtaaattttgttttcctcttcattttctgcatacgaaaagttcttttgggtatttgtgatttgtcatctccgaaacgtttctctctttaaaactttttttgaccgttctttataatttgaattgaaagagaagatgggaagagctgcgagatggttcaagggtatttttggtatgaagaagagcaaagagaaagagaactgtgtttccggcgacgttggaggtgaagccggtggttctaacattcaccggaaagttctccaagctgactccgtctggctcagaacttaccttgcggaaacagacaaagaacagaacaaacacgcgattgcggttgctgctgctacagccgcggctgctgacgcagcggttgcagcggctcaagctgctgtggcggtggtcaggttaacaagtaacggaagaagcggaggatattccgggaacgcaatggagcggtgggccgcagtgaaaattcaatcagtcttcaagggctatttggtaaatttcttaaaaacctccaaaacacttttttttttttttggtgtgtaatcgatttcgacgcaaaaagattgaattttgccatgtgggtatcgtttagattcgacaaaactaaaagaaagtatgtctgtactttacttgctccttacacatcttcgttaatgaaacagtacaacacatctttgctaatttcaagaaagttagattcttttctgattaaacactaaaatgtggttatttggtctaagtattttttttttttttgttataggcgagaaaagcgttacgagctttgaaaggtttagtgaagctacaagctttggtaagaggatacttagtccgcaaacgcgccgccgaaacgctgcatagcatgcaagctctcattagagctcaaaccagcgtccgatcgcaacgcatcaaccgcaacaacatgtttcatcctcgacactcacttgtaataaccattctttttcttttgtcttatttctaacataatctctatagtcaaaacttatgttttgatggattgatttgattataggagaggttggatgattcaagaagtgaaatccatagcaagagaatatcaatctctgtagagaaacagagtaatcacaacaacaatgcgtacgatgagaccagtcccaagattgtggagattgatacttacaagacgaaatcaagatcaaagagaatgaatgtggctgtatccgaatgtggagatgatttcatctatcaagccaaagatttcgaatggagttttccgggagagaaatgcaagtttcctacggctcaaaacacgccgagattctcttcatcaatggctaataataactattactacacgcccccatcgccggcgaaaagtgtttgcagagacgcttgttttaggccaagttatcctggtttgatgacacctagctatatggctaatacgcagtcgtttaaagccaaggtacgttcgcatagtgcaccgagacaacgtcctgatagaaaaagattgtcacttgatgagattatggcggctagaagtagcgttagtggtgtgaggatggtgcaaccacaaccacaaccgcaaacgcaaacgcagcaacagaaacgctctccttgttcgtatgatcatcagtttcgtcagaacgagactgattttagattctataattagtaaaaaacgttattttcgtccttaaagaaaatatcgtcatagcctttgacttttcatttatgactttccttttttttttttttttgtaaattattgctttgctttggaaaaaat

SEQ ID NO: 74 - DNA, 1170 bp, Arabidopsis thaliana; identical to SEQ ID NO: 14

SEQ ID NO: 75 - protein, 389 aa, Arabidopsis thaliana; identical to SEQ ID NO: 13

SEQ ID NO: 76 - DNA, 23 bp, Artificial Sequence
  Rice target sequence
cgcccccgccggacaggaagcgg

SEQ ID NO: 77 - DNA, 20 bp, Artificial Sequence
  Rice protospacer sequence
cgcccccgccggacaggaag

SEQ ID NO: 78 - DNA, 76 bp, Artificial Sequence; identical to SEQ ID NO: 27
  Full sgRNA nucleic acid sequence

SEQ ID NO: 79 - DNA, 23 bp, Artificial Sequence
  Arabidopsis CRISPR target sequence
aaagcgttacgagctttgaaagg

SEQ ID NO: 80 - DNA, 23 bp, Artificial Sequence
  Glycine max CRISPR target sequence
ctgacaagaaggagaagaaaagg

SEQ ID NO: 81 - DNA, 23 bp, Artificial Sequence
  Medicago truncatula CRISPR target sequence
tttcacctgcagaagctgcttgg

SEQ ID NO: 82 - DNA, 23 bp, Artificial Sequence
  Sorghum bicolor CRISPR target sequence
ggcgaccgagggctccgtgcggg

SEQ ID NO: 83 - DNA, 23 bp, Artificial Sequence
  Triticum aestivum CRISPR target sequence
tcgtgcggctcaccagcaaaggg

SEQ ID NO: 84 - DNA, 23 bp, Artificial Sequence
  Zea mays CRISPR target sequence
gacggcattcagacgcttcttgg

SEQ ID NO: 85 - DNA, 40 bp, Artificial Sequence
  crGSE5L-1 F
gacggccagtgccaagcttctcggatccactagtaacggc

SEQ ID NO: 86 - DNA, 41 bp, Artificial Sequence
  crGSE5L-1 R
cttcctgtccggcgggggcgacacaagcgacagcgcgcggg

SEQ ID NO: 87 - DNA, 41 bp, Artificial Sequence
  crGSE5L-2 F
cgcccccgccggacaggaaggttttagagctagaaatagca

SEQ ID NO: 88 - DNA, 40 bp, Artificial Sequence
  crGSE5L-2 R
cctgcaggcatgcaagcttcgacctcgagcggccgccagt

SEQ ID NO: 89 - DNA, 159 bp, Artificial Sequence
  Full sgRNA sequence in rice
gttttagagctagaaatagcaagttaaaataaggctagtccgttatcaacttgaaaaagtggcaccgagtcggtgctttttttgtcccttcgaagggcaattctgcagatatccatcacactggcggccgctcgaggtcgaagcttgcatgcctgcagg

SEQ ID NO: 90 - DNA, 24 bp, Artificial Sequence
  CRISPR target site WT
cgtggctcaggtcggtgtacgccg

SEQ ID NO: 91 - DNA, 23 bp, Artificial Sequence
  CRISPR target site gse5-cr
cgtggctcagtcggtgtacgccg

SEQ ID NO: 92 - DNA, 22 bp, Artificial Sequence
  ACTIN1 fwd primer
tgctatgtacgtcgccatccag

SEQ ID NO: 93 - DNA, 22 bp, Artificial Sequence
  ACTIN1 rev primer
aatgagtaaccacgctccgtca

SEQ ID NO: 94 - DNA, 19 bp, Artificial Sequence
  RT-GSE5 fwd primer
cgacgacacgaggagcgag

SEQ ID NO: 95 - DNA, 18 bp, Artificial Sequence
  RT-GSE5 rev primer
gagttggcgcaccagtcc

SEQ ID NO: 96 - DNA, 30 bp, Artificial Sequence
  RT-qSW5 fwd primer
agacggaggaggaggaacgggcggccagtg

SEQ ID NO: 97 - DNA, 28 bp, Artificial Sequence
  RT-qSW5 rev primer
gaatattcttcccagatccaggacgagg

SEQ ID NO: 98 - DNA, 36 bp, Artificial Sequence
  gGUS fwd primer
cggggcgtaatctagaatgttataccgccgttctgc

SEQ ID NO: 99 - DNA, 35 bp, Artificial Sequence
  gGUS rev primer
ggatcgatcctctagaccacctcctctgcaagaac

SEQ ID NO: 100 - DNA, 40 bp, Artificial Sequence
  gGFP fwd primer
ctagaggatccccgggtaccatgttataccgccgttctgc

SEQ ID NO: 101 - DNA, 41 bp, Artificial Sequence
  gGFP rev primer
tcattttttctaccggtaccggccacctcctctgcaagaac

SEQ ID NO: 102 - DNA, 34 bp, Artificial Sequence
  cGSE5 fwd primer
gcaggaattcaagcttatgggcaaggcggcgcgg

SEQ ID NO: 103 - DNA, 38 bp, Artificial Sequence
  cGSE5 rev primer
agtcactatggtcgactcaccacctcctctgcaagaac

SEQ ID NO: 104 - DNA, 36 bp, Artificial Sequence
  cGSE5L1 fwd primer
gcaggaattcaagcttatgggcaaggcggcgaggtg

SEQ ID NO: 105 - DNA, 38 bp, Artificial Sequence
  cGSE5L1 rev primer
agtcactatggtcgactcaccacctcctctgcaagaag

SEQ ID NO: 106 - DNA, 42 bp, Artificial Sequence
  pLUCL fwd primer
ctatgaccatgattacgaattcatgttataccgccgttctgc

SEQ ID NO: 107 - DNA, 35 bp, Artificial Sequence
  pLUCL rev primer
tttggcgtcttccatctcgccgctgtcgagcggtg

SEQ ID NO: 108 - DNA, 43 bp, Artificial Sequence
  pLUCM fwd primer
ctatgaccatgattacgaattcaagatcagggctagctagttc

SEQ ID NO: 109 - DNA, 35 bp, Artificial Sequence; identical to SEQ ID NO: 107
  pLUCM rev primer

SEQ ID NO: 110 - DNA, 42 bp, Artificial Sequence
  pLUCS fwd primer
ctatgaccatgattacgaattctcgcgccgctcctccttctg

SEQ ID NO: 111 - DNA, 35 bp, Artificial Sequence; identical to SEQ ID NO: 107
  pLUCS rev primer

SEQ ID NO: 112 - DNA, 36 bp, Artificial Sequence
  YN-736 fwd primer
cacgggggactctagatggtgagcaagggcgaggag

SEQ ID NO: 113 - DNA, 24 bp, Artificial Sequence
  YN-736 rev primer
ttccataggcatatactcttcctc

SEQ ID NO: 114 - DNA, 36 bp, Artificial Sequence
  YC-735 fwd primer
cacgggggactctagatggccgacaagcagaagaac

SEQ ID NO: 115 - DNA, 24 bp, Artificial Sequence
  YC-735 rev primer
cgcatagtcaggaacatcgtaagg

SEQ ID NO: 116 - DNA, 42 bp, Artificial Sequence
  ycGSE5 fwd primer
ccttacgatgttcctgactatgcgatgggcaaggcggcgcgg

SEQ ID NO: 117 - DNA, 37 bp, Artificial Sequence
  ycGSE5 rev primer
agtcactatggtcgatcaccacctcctctgcaagaac

SEQ ID NO: 118 - DNA, 45 bp, Artificial Sequence
  ynCaM fwd primer
gaggaagagtatatgcctatggaaatggcggaccagctcaccgac

SEQ ID NO: 119 - DNA, 37 bp, Artificial Sequence
  ynCaM rev primer
agtcactatggtcgatcacttggccatcatgaccttg

SEQ ID NO: 120 - DNA, 40 bp, Artificial Sequence; identical to SEQ ID NO: 85
  crGSE5-1 fwd primer

SEQ ID NO: 121 - DNA, 41 bp, Artificial Sequence
  crGSE5-1 rev primer
acctgagccacgccgcctcgacacaagcgacagcgcgcggg

SEQ ID NO: 122 - DNA, 41 bp, Artificial Sequence
  crGSE5-2 fwd primer
cgaggcggcgtggctcaggtgttttagagctagaaatagca

SEQ ID NO: 123 - DNA, 40 bp, Artificial Sequence; identical to SEQ ID NO: 88
  crGSE5-2 rev primer

SEQ ID NO: 124 - DNA, 28 bp, Artificial Sequence
  IDI1 fwd primer
cgtcttgcaaccaacgccgatgttatac

SEQ ID NO: 125 - DNA, 28 bp, Artificial Sequence
  IDI1 rev primer
gagcgtgtgtagggaaggagctgcatga

SEQ ID NO: 126 - DNA, 22 bp, Artificial Sequence
  IDI2 fwd primer
tccattttattggcatcactca

SEQ ID NO: 127 - DNA, 20 bp, Artificial Sequence
  IDI2 rev primer
cccaatcccaggctactgat